SEQUENCE LISTING (146 sequences)

SEQ ID NO: 1 - protein, 49 aa, synthetic construct
  REGION 1..49: note = Synthetic
DEEEKLRESLEEQKKRVKEIEREHEEIREALKRLQESVKRQEEALRQLG

SEQ ID NO: 2 - protein, 63 aa, synthetic construct
  REGION 1..63: note = Synthetic
SEQERREKAKEEVRELNEEFKEAEKRFRKLQEETKKALEEVEELNQRFEEALEEVERIKRRQK

SEQ ID NO: 3 - protein, 77 aa, synthetic construct
  REGION 1..77: note = Synthetic
DEEEEKKKEEKKKEERLKELREEWKRLRERLQELEEQHRELQEELKELREKWKRLKERLEELREKLRREEQRRREEK

SEQ ID NO: 4 - protein, 63 aa, synthetic construct
  REGION 1..63: note = Synthetic
PDSDELRKLLKQLEDLLQQLKDLIDKAERHNGDNDVILELHEIYLRLLDIYQKLAKIILKLLR

SEQ ID NO: 5 - protein, 77 aa, synthetic construct
  REGION 1..77: note = Synthetic
PQLDKFRDESRYLDKASKEVEDEIKKIEKIIRDADEKSLPEEALQDVVKSVVKVYLKSVKETIKSIELVIELIKVIM

SEQ ID NO: 6 - protein, 78 aa, synthetic construct
  REGION 1..78: note = Synthetic
ETEEIAYSSREAEKELKKIEEEQKKAKKELEEQKRQNGEPSSEISKQVLKLILKALKVIAKIQKSAQRQQDALIKILS

SEQ ID NO: 7 - protein, 72 aa, synthetic construct
  REGION 1..72: note = Synthetic
PEEEKLKELLKELKKVLDRLKKILERNDEEIKKSDELDDESLLEDIVELLKEIIKLWKILVELSDILLKLIS

SEQ ID NO: 8 - protein, 76 aa, synthetic construct
  REGION 1..76: note = Synthetic
PVDEIDKEVKKLEEEAKKSQEEVERLKQEVEKASKAGLDHEGDSRIFKKIHDVVTKQIKVIIRLIEVYVRLVEIIL

SEQ ID NO: 9 - protein, 75 aa, synthetic construct
  REGION 1..75: note = Synthetic
KQKEAIKVYLELLEVHSRVLKALIEQIKLFIELIKRPDEDLADKVRKSSEELKKIIKEVEKILRKVDDILYKVKS

SEQ ID NO: 10 - protein, 74 aa, synthetic construct
  REGION 1..74: note = Synthetic
PERELLELLIRLFNSQVKLFELQKELLELLRELLEENSEDNSRKLEDLLRKIQELLKENQDLLYTVEDVLRKLK

SEQ ID NO: 11 - protein, 75 aa, synthetic construct
  REGION 1..75: note = Synthetic
DEELIIYIIELLLKDDELLLKSLEIILRIIQLQLTSKDKDDGTDKIKKLVDESKKILDDSERLAEELRKIEEKLK

SEQ ID NO: 12 - protein, 68 aa, synthetic construct
  REGION 1..68: note = Synthetic
PKEEIRKLLKLLLRLNELLLKLHELLLRLLTNQGDHETTDEVVKRYDKIVKDYDKIVKDIKEIIDKLL

SEQ ID NO: 13 - protein, 75 aa, synthetic construct
  REGION 1..75: note = Synthetic
PEEKKEIKRTTEEVKEEFRKIQEEIKKLEEEAKKAEKGNGKEEIKELLLRLSELLARSLQLLAQQIEAIAKLIRG

SEQ ID NO: 14 - protein, 72 aa, synthetic construct
  REGION 1..72: note = Synthetic
SEVEKRLLEIHRRVAESHRILVEVHEALIEALRGNSEEAKEKLKELVKKLEKIIKEEEELLKKLEKIVKEAS

SEQ ID NO: 15 - protein, 73 aa, synthetic construct
  REGION 1..73: note = Synthetic
PDEEELEKLLRKIRELIKEIEEVIKEYQELKERGDGGEKEEKELIQANLRLLKLHTRLLKLYLELLKLIIKQG

SEQ ID NO: 16 - protein, 74 aa, synthetic construct
  REGION 1..74: note = Synthetic
DEKELAKTNAEILKTLLELFEAESRLQEIILKLLERPDEKEEDELKRALERLKEALERLEEAIKRYEETLKRLL

SEQ ID NO: 17 - protein, 70 aa, synthetic construct
  REGION 1..70: note = Synthetic
SEEDELEKAIREQKKALERLKEAIERYEETLKRGKNEKELAKTNAEILKTLLELFKSQLELAEIILKLLR

SEQ ID NO: 18 - protein, 76 aa, synthetic construct
  REGION 1..76: note = Synthetic
SEEDELRKLTEYQNRALEELEKAIKEYEKTLRRLLGAPDERDEKELAETNARILRTLLELFRTASEALDIILKLLR

SEQ ID NO: 19 - protein, 75 aa, synthetic construct
  REGION 1..75: note = Synthetic
SEKELIQLYVEAQELYAEAFELQVEIFKLLVKILSANSPKDEKEKIEKRIEEVQKRIKEIQERLQEIQKRLEEAG

SEQ ID NO: 20 - protein, 70 aa, synthetic construct
  REGION 1..70: note = Synthetic
DREEKLERETEKLEKKIRESQRRMEELRKQIETDPDEKELLEILIRSLRLLEEQLEALRKLNHAAQQLLG

SEQ ID NO: 21 - protein, 74 aa, synthetic construct
  REGION 1..74: note = Synthetic
SEREVIQRLIEVSEELSRATERLVEAHKIVLRAIASPNESEKEEIQKLAKELEEILKKLKKVIKESRELAEKAS

SEQ ID NO: 22 - protein, 72 aa, synthetic construct
  REGION 1..72: note = Synthetic
SKEEIEKELKKLKKELEELKREIEKSTKKLEEVRGSNSEKELIQIIKEQIEISERLLEISTRLLEIIIKLLG

SEQ ID NO: 23 - protein, 73 aa, synthetic construct
  REGION 1..73: note = Synthetic
DKEEKIKEILRRVQELRREHEELEERLKELKKELEKSPSQEELKKFIKLAQELIKLTLELIELSLRLIELLLG

SEQ ID NO: 24 - protein, 69 aa, synthetic construct
  REGION 1..69: note = Synthetic
DEEEVKEVAKEYREEVRKFKETIKEIRERIKRGSNEKEMIKLYVKLIELHTRLVKLHTKIVEILVKLLL

SEQ ID NO: 25 - protein, 69 aa, synthetic construct
  REGION 1..69: note = Synthetic
SEDEDLIRSVRVIQKSVEIILEMVKIIQELLKSTSEEELKELLKKLKRLIEKLEELLRQYEELLKKIKG

SEQ ID NO: 26 - protein, 76 aa, synthetic construct
  REGION 1..76: note = Synthetic
SKEEILKLHLEVAEIHIETVKIYLEVIKILIRLIEKNSSKEDERELEEELKELERELEELQKKLEEIRKELQEIEG

SEQ ID NO: 27 - protein, 76 aa, synthetic construct
  REGION 1..76: note = Synthetic
SREETIIQFTKLLIKFNSLLVKHLKLIIRFIELEENNSSKESEKEILEESKRIVERLEEIVRELQKLSKRLEEIIG

SEQ ID NO: 28 - protein, 72 aa, synthetic construct
  REGION 1..72: note = Synthetic
SEEEELKKLQEEIKKLKKQVERLEKESKELEKRLEKSPSEEIAKELLRITAELVEISEELAEATKRLLEIAG

SEQ ID NO: 29 - protein, 70 aa, synthetic construct
  REGION 1..70: note = Synthetic
SELEEEIKRIRKEIEESRRRIEELRKKAQKAREGSSEKEFLQIYIELQKVLIEQIKIYIELLQIIFELQS

SEQ ID NO: 30 - protein, 70 aa, synthetic construct
  REGION 1..70: note = Synthetic
SEKEEAEKKAREVKKRIEEQEKESKKVKEEAKKLGPDTEVLKILIEVHELHIESLRVVAEITQALVEILK

SEQ ID NO: 31 - protein, 109 aa, synthetic construct
  REGION 1..109: note = Synthetic
DESLLEDIVELLKEIIKLWKILVELSDILLKLIGGDDKVRKSSEELKKIIKEVEKILRKVDDILYKVSAPSEEKQKEAIKVYLELLEVHSRVLKALIEQIKLFIELIKR

SEQ ID NO: 32 - protein, 111 aa, synthetic construct
  REGION 1..111: note = Synthetic
PDEIDKEVKKLEEEAKKSQEEVERLKQEVEEASKAGLDHEADSRIFKKIHDVVTKQIKVIIRLIEVYVRLVEIILGSGSDEKLKELLKELKKVLDRLKKILERNDEEIKKS

SEQ ID NO: 33 - protein, 112 aa, synthetic construct
  REGION 1..112: note = Synthetic
DHEADSRIFKKIHDVVTKQIKVIIRLIEVYVRLVEIILGSGSDEKLKELLKELKKVLDRLKKILERNDEEIKKSDELDDESLLEDIVELLKEIIKLWKILVELSDILLKLID

SEQ ID NO: 34 - protein, 119 aa, synthetic construct
  REGION 1..119: note = Synthetic
HEELADKVRKSSEELKKIIKEVEKILRKVDDILEKVSAPSEEKQKEAIKVYLELLEVHSRVLKALIEQIKLFIELIKRKVKRVPDEIDKEVKKLEEEAKKSQEEVERLKQEVEEASKAG

SEQ ID NO: 35 - protein, 150 aa, synthetic construct
  REGION 1..150: note = Synthetic
DESLLEDIVELLKEIIKLWKILVELSDILLKLIGGDDKVRKSSEELKKIIKEVEKILRKVDDILYKVSAPSEEKQKEAIKVYLELLEVHSRVLKALIEQIKLFIELIKRKHKRVPDEIDKEVKKLEEEAKKSQEEVERLKQEVEEASKAG

SEQ ID NO: 36 - protein, 74 aa, synthetic construct
  REGION 1..74: note = Synthetic
DHEADSRIFKKIHDVVTKQIKVIIRLIEVYVRLVEIILGSGSDEKLKELLKELKKVLDRLKKILERNDEEIKKS

SEQ ID NO: 37 - protein, 146 aa, synthetic construct
  REGION 1..146: note = Synthetic
DHEADSRIFKKIHDVVTKQIKVIIRLIEVYVRLVEIILGSGSDEKLKELLKELKKVLDRLKKILERNDEEIKKSDELDDESLLEDIVELLKEIIKLWKILVELSDILLKLIGGDDKVRKSSEELKKIIKEVEKILRKVDDILYKVS

SEQ ID NO: 38 - protein, 76 aa, synthetic construct
  REGION 1..76: note = Synthetic
QKEAIKVYLELLEVHSRVLKALIEQIKLFIELIKRKHKRVPDEIDKEVKKLEEEAKKSQEEVERLKQEVEEASKAG

SEQ ID NO: 39 - protein, 34 aa, synthetic construct
  REGION 1..34: note = Synthetic
QKEAIKVYLELLEVHSRVLKALIEQIKLFIELIK

SEQ ID NO: 40 - protein, 191 aa, synthetic construct
  REGION 1..191: note = Synthetic
PVDEIDKEVKKLEEEAKKSQEEVERLKQEVEKASKAGLDHEGDSRIFKKIHDVVTKQIKVIIRLIEVYVRLVEIILGSGSDEKLKELLKELKKVLDRLKKILERNDEEIKKSDELDDESLLEDIVELLKEIIKLWKILVELSDILLKLIDRDTHEDLADKVRKSSEELKKIIKEVEKILRKVDDILYKVKS

SEQ ID NO: 41 - protein, 37 aa, synthetic construct
  REGION 1..37: note = Synthetic
HEELADEVRKSSEELKKIIEEVEKILRKVDDILYKVG

SEQ ID NO: 42 - protein, 36 aa, synthetic construct
  REGION 1..36: note = Synthetic
QKEAIKVYLELLEVHSRVLKALIEQIKLFIELIKRK

SEQ ID NO: 43 - protein, 147 aa, synthetic construct
  REGION 1..147: note = Synthetic
PDEIDKEVKKLEEEAKKSQEEVERLKQEVEEASKAGLDHEADSRIFKKIHDVVTKQIKVIIRLIEVYVRLAEIIGEPSEKLKELLKELKKVLDRLKKILERNDEEIKKSDELDDESLLEDIVELLKEIIKLWKILVELSDILLKLID

SEQ ID NO: 44-46 - skipped (3 entries)

SEQ ID NO: 47 - protein, 38 aa, synthetic construct
  REGION 1..38: note = Synthetic
DEDLADKVRKSSEELKKIIKEVEKILRKVDDILYKVKS

SEQ ID NO: 48 - protein, 171 aa, synthetic construct
  REGION 1..171: note = Synthetic
DEEEKLRESLEEQKKRVKEIEREHEEIREALKRLQQSVLRQAVRLAKLGDDSEMINRIVKDLAEQAKEATDKREVIKIVKALAELAKNSTDSELVNEIVKQLAEVAKEATDKELVIYIVKILAELAKQSTDSELVNEIVKQLEEVAKEATDKELVEHIEKILEELKKQSTD

SEQ ID NO: 49 - protein, 227 aa, synthetic construct
  REGION 1..227: note = Synthetic
SEQERREKAKEEVRELNEEFKEAEKRFRKLQEETKKALEEVEELNQRFQEALTEVAAKKLALEISRVIKTLKESGSSYEEIAEIVARIVAEIVEALKRSGASEKDIATIVAAIISAVIQTLKESGSSYEVIAEIVARIVAEIVEALKRSGTSEDEIAEIVARVISEVIRTLKESGSSYEVIKEIVQRIVEEIVEALKRSGTSEDEINEIVRRVKSEVERTLKESGSS

SEQ ID NO: 50 - protein, 402 aa, synthetic construct
  REGION 1..402: note = Synthetic
MSSDEEEARELIERAKEAAERAQEAAERTGDPRVRELARELKRLAQEAAEEVKRDPSSSDVNEALKLIVEAIEAAVRALEAAERTGDPEVRELARELVRLAVEAAEEVQRNPSSSDVNEALKLIVEAIEAAVRALEAAERTGDPEVRELARRMVRAAVRAAEVVQRNPSDEEANEMLKQIVKLAQQAVEMLREAEESGDPEKREKAKQTVEQAIREAELLLLIWEWKRLRERLQELEEQHRELQEELKELREKWKRLKERLERLRLELRLEEMARRARNATDSEQINEIVKQLAEIAKEATDKRAVIKIVKILAELAKKSTDSELVNEIVKQLAEVAKEATDKELVIYIVKILAELAKQSTDSELVNEIVKQLEEVAKEATDKELVEHIEKILEELKKQSTD

SEQ ID NO: 51 - skipped

SEQ ID NO: 52 - protein, 236 aa, synthetic construct
  REGION 1..236: note = Synthetic
MSSDEEEARELIERAKEAAERAQEAAERTGDPRVRELARELKRLAQEAAEEVKRDPSSSDVNEALKLIVEAIEAAVRALEAAERTGDPEVRELARELVRLAVEAAEEVQRNPSSSDVNRALKLIVRAIEAAVQLLEHAEKSGDPEVRELARELVRRLVEAAEEVQRNPSDEQKNTQLRRLIAKAEVWLLNEEFKEAEKRFRKLQEETKKALEEVEELNQRFEEALEEVERIKRRQK

SEQ ID NO: 53 - skipped

SEQ ID NO: 54 - protein, 202 aa, synthetic construct
  REGION 1..202: note = Synthetic
SEKEKVEELAQRIREQLPDTELAREAQELADEARKSDDSEALKVVYLALRIVQQLPDTELAREALELAKEAVKSTDSEALKVVYLALRIVQQLPDTELARLALELAKKAVEMTAQEVLEIARAALKAAQAFPNTELAELMLRLAEVAARVMKELERNDEEIKKSDELDDESLLEDIVELLKEIIKLWKILVEVSDVMLKLIS

SEQ ID NO: 55 - protein, 76 aa, synthetic construct
  REGION 1..76: note = Synthetic
PVDEIDKEVKKLEEEAKKSQEEVERLKQEVEKASKAGLDHEGDSRIFKKIHDVVTKQIKVILRLIAVYAELVAIIG

SEQ ID NO: 56-57 - skipped (2 entries)

SEQ ID NO: 58 - protein, 198 aa, synthetic construct
  REGION 1..198: note = Synthetic
SEKEKVEELAQRIREQLPDTELAREAQELADEARKSDDSEALKVVYLALRIVQQLPDTELAREALELAKEAVKSTDSEQLEVVRLALEIVQLAPDTRLARAALKLAKEAVKSTDQEELKKVKAILRVASEVLKLEEEAKKSQEEVERLKQEVEKASKAGLDHEGDSRIFKKIHDVVTKQIKVIIRLIEVYVRLVEIIL

SEQ ID NO: 59 - protein, 75 aa, synthetic construct
  REGION 1..75: note = Synthetic
KQKEAIKVYLELLEVHSRVLKALIEQIKLFIELIMEPDEDLADKVRKSSEELKKIIKEVEKILRKVDDILEKVKS

SEQ ID NO: 60 - protein, 316 aa, synthetic construct
  REGION 1..316: note = Synthetic
SEKEKVEELAQRIREQLPDTELAREAQELADEARKSDDSEALKVVYLALRIVQQLPDTELAREALELAKEAVKSTDSEALKVVELALKIVQQLPDTELAKEALKLAKEAVKSTDSEALKVVELALEIVQQLPDTELAKEALELAEEAVKSTDSEALKVVKLALEIVQQLPDTELAREALELAKEAVKSTDSEALKVVYLALRIVQQLPDTELARLALELAKKAVEMTAQEVLEIARAALKAAQAFPNTELAELMLRLAEVAARVMKELERNDEEIKKSDELDDESLLEDIVELLKEIIKLWKILVEVSDVMLKLIS

SEQ ID NO: 61 - protein, 312 aa, synthetic construct
  REGION 1..312: note = Synthetic
SEKEKVEELAQRIREQLPDTELAREAQELADEARKSDDSEALKVVYLALRIVQQLPDTELAREALELAKEAVKSTDSEALKVVELALKIVQQLPDTELAKEALELAKEAVKSTDSEALKVVELALEIVQQLPDTELAKEALKLAKEAVKSTDSEALKVVYLALRIVQQLPDTELAREALELAKEAVKSTDSEQLEVVRLALEIVQLAPDTRLARAALKLAKEAVKSTDQEELKKVKAILRVASEVLKLEEEAKKSQEEVERLKQEVEKASKAGLDHEGDSRIFKKIHDVVTKQIKVILRLIAVYAELVAIIG

SEQ ID NO: 62 - skipped

SEQ ID NO: 63 - protein, 202 aa, synthetic construct
  REGION 1..202: note = Synthetic
SEKEKVEELAQRIREQLPDTELAREAQELADEARKSDDSEALKVVYLALRIVQQLPDTELAREALELAKEAVKSTDSEALKVVYLALRIVQQLPDTELARLALELAKKAVEMTAQEVLEIAKAALKAAQAFPNTELAELMLRLAEVAARVMKELERNDEEIKKSDELDDESLLEDIVELLKEIIKLWKILVEVSDVMLKLIS

SEQ ID NO: 64 - protein, 336 aa, synthetic construct
  REGION 1..336: note = Synthetic
TEEKIAKEISRIAEESKKRIEELARKADNKTTETEVDKAIEKIAKLAREAIKRIEDLAKNLASEEFMARAISAIAELAKKAIEAIYRLADNHTTDTFMAKAIEAIAELAKEAIKAIADLAKNHTTEEFMARAISAIAELAKKAIEAIYRLADNHTTDTFMAKAIEAIAELAKEAIKAIADLAKNHTTEEFMAKAISAIMELAVKAILAIARLASNHTSQTYREKAREAVEKIARTAEKAIEDLAKNITTEEYKERAKKARAIVRVMAEVAKLLIEAVESQEEVERLKQEVEKASKAGLDHEGDSRIFKKIHDVVTKQIKVIIRLIAVYAELVAIIG

SEQ ID NO: 65 - protein, 75 aa, synthetic construct
  REGION 1..75: note = Synthetic
KQKEAIKVYLELLEVHSRVLKALIEQILLFIELIVRPDEDLADKVRKSSEELKKIIKEVEKILRKVDDILYKVKS

SEQ ID NO: 66 - protein, 202 aa, synthetic construct
  REGION 1..202: note = Synthetic
SEKEKVEELAQRIREQLPDTELAREAQELADEARKSDDSEALKVVYLALRIVQQLPDTELAREALELAKEAVKSTDSEALKVVYLALRIVQQLPDTELARLALELAKKAVEMTAQEVLAIAKAALKAAQAFPNTELAELMLRLAEVAARVMKELERNDEEIKKSDELDDESLLEDIVELLKEIIKLWKILVEVSDVMLKLIS

SEQ ID NO: 67 - protein, 219 aa, synthetic construct
  REGION 1..219: note = Synthetic
SEEVNERVKQLAEKAKEATDKEEVIEIVKELAELAKQSTDSELVNEIVKQLAEVAKEATDKELVIYIVKILAELAKQSTDSELVNEIVKQLAEVAKEATDKELVKYIAEILDELAKQSTDDELRKEIIKQMLEVAAEAKDKELIEDLVKIMVDMLEELAKSSQEEVERLKQEVEKASKAGLDHEGDSRIFKKIHDVVTKQIKIIILLIAVYAMLVAAIG

SEQ ID NO: 68-69 - skipped (2 entries)

SEQ ID NO: 70 - protein, 224 aa, synthetic construct
  REGION 1..224: note = Synthetic
NDEKRKRAEKALQRAQEAEKKGDVEEAVRAAQEAVRAAKESGDNDVLRKVAEQALRIAKEAEKQGNVEVAVKAARVAVEAAKQAGDNDVLRKVAEQALRIAKEAEKQGNAEVAAKAAKVAAEAAAQAGDDDVLKKVIEVANRIAKVANKLGDIKTAVEALEIAAEASQVMVELLKQEVEKASKAGLDHEGDSRIFKKIHEVVTIQIKIIIILIAVYALLVAIIG

SEQ ID NO: 71 - skipped

SEQ ID NO: 72 - protein, 202 aa, synthetic construct
  REGION 1..202: note = Synthetic
SEKEKVEELAQRIREQLPDTELAREAQELADEARKSDDSEALKVVYLALRIVQQLPDTELAREALELAKEAVKSTDSEALKVVYLALRIVQQLPDTELARLALELAKKAVEMTAQEVLAIAKAALKAAQAFPNTELAELMLRLAEVAARVMKELERNDEEIKKSDELDDASLLADIVRLLAEIIKLWKILVEVSDVMLKLIS

SEQ ID NO: 73 - protein, 198 aa, synthetic construct
  REGION 1..198: note = Synthetic
SEKEKVEELAQRIREQLPDTELAREAQELADEARKSDDSEALKVVYLALRIVQQLPDTELAREALELAKEAVKSTDSEQLEVVRLALEIVQLAPDTRLARAALKLAKEAVKSTDQEELKKVKAILRVASEVLKLEEEAKKSQEEVERLKQEVEKASKAGLDHEGDSRIFKKIHDVVTKQIKVILRLIAVYAELVAIIG

SEQ ID NO: 74 - protein, 213 aa, synthetic construct
  REGION 1..213: note = Synthetic
KQKEAIKVYLELLEVHSRVLKALIEQIKLFIELIMEPDEDLADKVRKSSEELKKIIKEVEKILRKVELKLRIDKARKLVRERPGSNLAKKALEEMLRAAEEAAKLPDPEALKQAVKAAEEVVREQPGSNLAKKALEIILRAAEELAKLPDPEALKEAVKAAEKVVREQPGSELAKKALEIIERAAEELKKSPDPEAQKEAKKAEQKVREERPG

SEQ ID NO: 75-79 - skipped (5 entries)

SEQ ID NO: 80 - protein, 260 aa, synthetic construct
  REGION 1..260: note = Synthetic
KQKEAIKVYLELLEVHSRVLKALIEQIKLFIELIMEPDEDLADKVRKSSEELKKIIKEVEKILKKVRKILIRLKARRVEKEAKELAKELDSEEAKKVVERIKEAAEAAERAAEQGKDEVAELALKVLREAIELAKENRSEEALKVVLEIARAALAAAQAAEEGKTEVAKLALKVLEEAIELAKENRSEEALKVVLEIARAALAAAQAAEEGKSDEARDALRRLEEAIEEAKENRSKESLEKVREEAKEAEQQAEDAREGK

SEQ ID NO: 81-82 - skipped (2 entries)

SEQ ID NO: 83 - protein, 235 aa, synthetic construct
  REGION 1..235: note = Synthetic
KQKEAIKVYLELLEVHSRVLKALIEQIKLFIELIMEPDEDLADKVRKSSEELKKIIKEVEKILKKVSQIVAELALKRAREAEKKGDVEEAVRAAQKAVKAAKDAGDNDMLRKVAEVALRIAKEAEKQGNVEVAVKAARVAVEAAKQAGDNDVLRKVAEQALRIAKEAEKQGNVEVAVKAARVAVEAAKQAGDQDVLRKVSEQAERISKEAKKQGNSEVSEEARKVADEAKKQTGD

SEQ ID NO: 84 - protein, 4 aa, synthetic construct
  REGION 1..4: note = Synthetic
AAAA

SEQ ID NO: 85 - protein, 4 aa, synthetic construct
  REGION 1..4: note = Synthetic
AAAA

SEQ ID NO: 86 - protein, 289 aa, synthetic construct
  REGION 1..289: note = Synthetic
KQKEAIKVYLELLEVHSRVLKALIEQIKLFIELIMEPDEDLADKVRKSSEELKKIIEEVKQILRLVEAELMRQKAEEAIKKARKTGDPELLRKALELLEEAVRIVEEAIKKDPDDDEAVELAVRLARMLKKVAEELQERAKKTGDPELLKLALRALEVAVRAVELAIKSNPDNDEAVETAVRLARELKKVAEELQERAKKTGDPELLKLALRALEVAVRAVELAIKSNPDNEEAVETAKRLAEELRKVAELLEERAKETGDPELQELAKRAKEVADRARELAKKSNPNN

SEQ ID NO: 87 - protein, 225 aa, synthetic construct
  REGION 1..225: note = Synthetic
NDEKRKRAEKALQRAQEAEKKGDVEEAVRAAQEAVRAAKESGDNDVLRKVAEQALRIAKEAEKQGNVEVAVKAARVAVEAAKQAGDNDVLRKVAEQALRIAKEAEKQGNVKVAADAVKVAVEAATQAGDQDVLRKASEQAKKVAKEAKKKGDHKVAVKATTLAVKAEFAAIQEEIKKLEEEAKKAEKGNGKEEIKELLLRLSELLARSLQLLAQQIEAIAKLIRG

SEQ ID NO: 88 - protein, 72 aa, synthetic construct
  REGION 1..72: note = Synthetic
SEVEKRLLEIHRRVAESHRILVEVHEALIEALRGNSEEAKEKLKELVKELEKIIKEEEELLKELEKIVEEAS

SEQ ID NO: 89 - protein, 73 aa, synthetic construct
  REGION 1..73: note = Synthetic
PDEEELEKLLRKIRELIKEIEEVIKEYQKLKERGDGGEKEEKELIQANLRLLKLHTRLLKLYLELLKLIIKQG

SEQ ID NO: 90-91 - skipped (2 entries)

SEQ ID NO: 92 - protein, 212 aa, synthetic construct
  REGION 1..212: note = Synthetic
NDEKRKRAEKALQRAQEAEKKGDVEEAVRAAQEAVRAAKESGDNDVLRKVAEQALRIAKEAEKQGNVEVAVKAARVAVEAAKQAGDNDVLRKVAEQALRIAEEALKQGNTEVAKKANRVAREAAKQAGDQDVLRKVEKMELKIRLAELLRKIRELIKEIEEVIKEYQKLKERGDGGEKEEKELIQANLRLLKLHTRLLKLYLELLKLIIRMG

SEQ ID NO: 93 - skipped

SEQ ID NO: 94 - protein, 201 aa, synthetic construct
  REGION 1..201: note = Synthetic
SEVEKRLLEIHREVAESHRILVEVHEALIEALRGNSEEAKEKLKELVKKLEKIIKEEEELLKKLEKIVVQALAKAAKQATDSRQVNEIVKQLAEIAKEATDKRLVIEIVKILAELAKQSTDSELVNEIVKQLAEVAKEATDKELVIYIVKILAELAKQSTDSELVNEIVKQLEEVAKEATDKELVEHIEKILEELKKQSTD

SEQ ID NO: 95 - protein, 73 aa, synthetic construct
  REGION 1..73: note = Synthetic
PDEEELEKLLRKIRELIKEIEEVIKEYQELKERGDGGEKEEEELIQANLRLLKLHTRLLKLYLELLKLIIKQG

SEQ ID NO: 96 - skipped

SEQ ID NO: 97 - protein, 255 aa, synthetic construct
  REGION 1..255: note = Synthetic
SETEKRLLEIHRRVAESHRILVEVHEALIEALRGNSEEAKEKLKELVKKLEKIIREEEKLTVELAAKVAKSADDSEKVNEIVKTLAEIAKEATDKEQVIRIVKILAELAKQSTDSELVNEIVKQLAEVAKEATDKELVIYIVDILLKLAEQADDDELVEEIRKQLEEVAKEATDKELVEIIKAVIVLLVIISVVARMGVTMEIHKSGREVKVVIKGLHESQQEQLLEAVLRAAEEAGVRVRIRFKGDTVTIVVRG

SEQ ID NO: 98 - protein, 73 aa, synthetic construct
  REGION 1..73: note = Synthetic
PDEEELEKLLRKIRELIKEIEEVIKEYQELKERGDGGEKEEKELIQANLRLLKLHTELLKLYLELLKLIIKQG

SEQ ID NO: 99 - protein, 225 aa, synthetic construct
  REGION 1..225: note = Synthetic
NDEKRKRAEKALQRAQEAEKKGDVEEAVRAAQEAVRAAKESGDNDVLRKVAEQALRIAEEAEKQGNVEVAVKAARVAVEAAKQAGDNDVLRKVAEQALRIAEEAEKQGNVKVAADAVEVAVEAATQAGDQDVLRKASEQAKKVAKEAKKKGDHKVAVKATTLAVKAEFAAIQEEIKKLEEEAKKAEKGNGKEEIKELLLELSELLARSLQLLAQQIEAIAKLIRG

SEQ ID NO: 100 - protein, 226 aa, synthetic construct
  REGION 1..226: note = Synthetic
SEVEKRLLEIHRQVAESHRILVEVHEALIEALRGNSEEAKEKLKELVKKLEKIIRQEELLLQLLEALVKALQAKKEGDPELVLEAAKDALRVAEQAAKEGDKEMFKQAAELALWLAKQLVEVASKEGDPELVLEAAKVALRVAELAAKNGDKEVFKKAAESALEVAKRLVEVASKEGDPELVEEAAKVAEEVRKLAKKQGDEEVYEKARETAREVKEELKRVREEK

SEQ ID NO: 101 - protein, 212 aa, synthetic construct
  REGION 1..212: note = Synthetic
NDEKRKRAEKALQRAQEAEKKGDVEEAVRAAEEAVRAAKESGDNDVLRKVAEQALRIAKEAEKQGNVEVAVKAARVAVEAAKQAGDNDVLRKVAEQALRIAEEALKQGNTEVAKEANRVAREAAKQAGDQDVLRKVEKMEEKIELAELLEKIRELIKEIEEVIKEYQKLKERGDGGEKEEKELIQANLRLLKLHTELLKLYLELLKLIIRQG

SEQ ID NO: 102 - protein, 399 aa, synthetic construct
  REGION 1..399: note = Synthetic
DEEEKLRESLEEQKKRVKEIEREHEEIREALKRLQQSVLRQAVRLAKLGDDSEMIRRIVKDLMQQAIEATDEREVEKIKKAAEELSKNSTDEDLKKQIKAVQELVKAVETVREAKKQGDPKKVLKAAEIAMEVATWAAERNDEMIFKLAAKMAKEVAERLKEVAKKENDSALELAAEAIKAAVEALEAALRTGDPRVRELAKELVRLAKEAAEEATRDSKDSDKNEALKLIVEAIKAAVRALEAAERTGDPEVRELARELVRLAVEAAEEVLRNPSSSDVNRALKLIVRAIEAAVQLLEHAEKSGDPEVRELARELVRRLVEAAEEVQRNPSDEQKNTQLRRLIAKAEVWLLNEEFKEAEKRFRKLQEETKKALEEVEELNQRFEEALEEVERIKRRQK

SEQ ID NO: 103-104 - skipped (2 entries)

SEQ ID NO: 105 - protein, 464 aa, synthetic construct
  REGION 1..464: note = Synthetic
DEEEKLRESLEEQKKRVKEIEREHEEIREALKRLQQSVLRQAVRLAKLGDDSEMIQRIVEDLARQAMEATDDREVEKIEKALDELAKNSTDEELKKVIKVAKTLVRVAKLVKEAEELLRQAKEKGSEEDLEKALRTAEEAAREAKKVLEEAEKAQIAEAALLAVELVVRVAELLLRIAKESGSEEALERALRVAEEAARLAKRVLELAEKQGDPEVALRAVELVVRVAELLLRIAKESGSSEALTRAWRVAIEAFQLAVRVLKLAQKQNDSEVARRATELMKRVAELMERIARESGSSAFKAAAEALEAAARTNDKEVVDLARELARLAIEALEEVERNSKSSDVNRALKLILRAIKAAVQLLEHAEKSGDPEVRELARELVRRLVEAAEEVQRNPSDEQKNTQLRRLIAKAEVWLLNEEFKEAEKRFRKLQEETKKALEEVEELNQRFEEALEEVERIKRRQK

SEQ ID NO: 106 - protein, 360 aa, synthetic construct
  REGION 1..360: note = Synthetic
MGDEEEKLRESLEEQKKRVKEIEREHEEIREALKRLQQSVLRQAVRLAKLGDDSRMIASIIRDLVKQALRATDEREQKKIKKALEELAQNATDEALKLLIEAAKSLLRAAEASERGDEEEFRKAAEKALELAKRLVEIAKKQSLPEFVLAAAEIALAVAELAAKNNDSEVLEKALRSAAEVAQRLLEVAEKENNQELVEEAAKILKRALEIVEKAAKKLKERNDQSSDVNRALTLIRRAQEAADQLLEHARKSGDPEVWELAIRLVARLVEAAAKVAANMSDEQQNTQLRRLIAKAEVWLLNEEFKEAEKRFRKLQEETKKALEEVEELNQRFEEALEEVERIKRRQKGGSGWSHPQFEK

SEQ ID NO: 107 - skipped

SEQ ID NO: 108 - protein, 515 aa, synthetic construct
  REGION 1..515: note = Synthetic
DEEEKLRESLEEQKKRVKEIEREHEEIREALKRLQQSVLRQAVRLAKLGDDSEMINRIVKDLAEQAKEATDKREVIKIVKALAELAKNSTDRELINEIAKQLAEVAKEATDEELQRYIAKILIELAEQAIKEAERSLREGNPEKAREDVRLALELVRLLLEIAQALNLLEVLEEAARLAFEVARVAKEVGSPETARQARETAERLLEWAMKMIEEKAKKEKDRNDQSSDVNEAAKLIREAAEAAQRAREAASRTGDPEVKDLAWRMVEAALKAAAVVATNPSDEEANEMLKQIVKLAQQAVEMLREAEESGDPEKREKAKQTVEQAIREAELLLLIWEWKRLRERLQELEEQHRELQEELKELREKWKRLKERLERLRLELRLEEMARRARNATDSEQINEIVKQLAEIAKEATDKRAVIKIVKILAELAKKSTDSELVNEIVKQLAEVAKEATDKELVIYIVKILAELAKQSTDSELVNEIVKQLEEVAKEATDKELVEHIEKILEELKKQSTD

SEQ ID NO: 109 - skipped

SEQ ID NO: 110 - protein, 358 aa, synthetic construct
  REGION 1..358: note = Synthetic
TTNFHLINGSEEARQLIQKAVEAISKKEGTEVHFEKSDGTLEIRVKNLHPRQEDLIKKFIEALLLALVAKGELEQAEKEGDAEVALRAVEKVVRVAELLLRLAKEAGSEEALKAALEIAEQAARLAKRVLELAEKQGDAEVALKAVLLVVIVAALLVKIAKESGSEEAKERATRVAREASRLADRVEELARKQNNEDVTLAAKVLKLAADIIAQLMNTELAEEAAQLATEALKATDREQLEVVRLALEIVQLAPDTRLARAALKLAKEAVKSTDQEELKKVKAILRVASEVLKLEEEAKKSQEEVERLKQEVEKASKAGLDHEGDSRIFKKIHDVVTKQIKVILRLIAVYAELVAIIG

SEQ ID NO: 111 - protein, 314 aa, synthetic construct
  REGION 1..314: note = Synthetic
NTHFIVVHGGEEARQLAETAVREISKKEGTEVRFEKKDGLLSIEVKNLSEELQRLIQELLQLLVRLAALLEAVRAVEEAIKRNPDNDEAVETAVRLARELKKVAELLQELAKKAGVPAILRGALLALEVAVRAVELAIKSNPDNDEAVETAVRLAVVKLALEIVQQLPDTELAREALELAKEAVKSTDSEALKVVYLALRIVQQLPDTELARLALELAKKAVEMTAQEVLEIARAALKAAQAFPNTELAELMLRLAEVAARVMKELERNDEEIKKSDELDDESLLEDIVELLKEIIKLWKILVEVSDVMLKLIS

SEQ ID NO: 112 - skipped

SEQ ID NO: 113 - protein, 409 aa, synthetic construct
  REGION 1..409: note = Synthetic
SSIFLLSNVDESARQLAEELVREISKKEGTEVRFEKDDGFLTIEVKNLSEERLREIARALQLIVDVANAERVVRERPGSNLAKKALEIILRAAEELAKLTLEASLKAAQIAAELVVREQPGSNLAEKAKTIILQARAAELAIRIEEQLKDTELAKEAKELATEAIKAQDKEALKVVELALKIVQQLPDTELAKEALKLAKEAVKSTDSEALKVVELALEIVQQLPDTELAKEALELAEEAVKSTDSEALKVVKLALEIVQQLPDTELAREALELAKEAVKSTDSEALKVVYLALRIVQQLPDTELARLALELAKKAVEMTAQEVLEIARAALKAAQAFPNTELAELMLRLAEVAARVMKELERNDEEIKKSDELDDESLLEDIVELLKEIIKLWKILVEVSDVMLKLIS

SEQ ID NO: 114 - protein, 463 aa, synthetic construct
  REGION 1..463: note = Synthetic
TWQWVLINISEEARQLIEKAVRAISKKEGTEVHFEKDDGVLHIRVKNLHEKRAREIHKVAKLILEVAAAERIVRERPGSNLAKKALEIILRAAEELAKADVDAALEAAVRAAEKVVREQPGSNLAKKALEIILRAAEELAKLPDPEALKEAVKAAEKVVREQPNSELAKKAREIIKRAAEELAKSPDKEAIQEAATALAILIELILSNTELAREAKELATEARKATDSEAAKVVFLALFIVVQLPDTELAKEALELAKEAVKSTDSEALKVVELALEIVQQLPDTELAKEALKLAKEAVKSTDSEALKVVYLALRIVQQLPDTELAREALELAKEAVKSTDSEQLEVVRLALEIVQLAPDTRLARAALKLAKEAVKSTDQEELKKVKAILRVASEVLKLEEEAKKSQEEVERLKQEVEKASKAGLDHEGDSRIFKKIHDVVTKQIKVILRLIAVYAELVAIIG

SEQ ID NO: 115 - skipped

SEQ ID NO: 116 - protein, 346 aa, synthetic construct
  REGION 1..346: note = Synthetic
MGNTHFIVVHGGEEARQLAETAVREISKKEGTEVRFEKKDGLLSIEVKNLSEELQRLIQELLQLLVRLAALLEAVRAVEEAIKRNPDNDEAVETAVRLARELKKVAELLQELAKKAGVPAILRGALLALLVAARAVALAALANSKNDEAREALKRLAEELKKVAKELKERAKKTKDSELERLALAAELMALAAEIVAQLANTELAREALELATEAIKASDDEALRVVKLALRIVQQLPDTELARLALELAKKAVEMTAQEVLEIARAALKAAQAFPNTELAELMLRLAEVAARVMKELERNDEEIKKSDELDDESLLEDIVELLKEIIKLWKILVEVSDVMLKLIS

SEQ ID NO: 117 - protein, 375 aa, synthetic construct
  REGION 1..375: note = Synthetic
MGTTNFHLINGSEEARQLIQKAVEAISKKEGTEVHFEKSDGTLEIRVKNLHPRQEDLIKKFIEALLLALVAKGELEQAEKEGDAEVALRAVEKVVRVAELLLRLAKEAGSEEALKAALEIAEQAARLAARVFTLAVKQNDSDVAKRAVELIKRVAELLKRIAKESGSEEAKERAEKVEKAAEVAEIAARILDQLSNTELAKEALKLASEALKSDIKEALEVVRLALRIVQQLPDTELAREALELAKEAVKSTDSEQLEVVRLALEIVQLAPDTRLARAALKLAKEAVKSTDQEELKKVKAILRVASEVLKLEEEAKKSQEEVERLKQEVEKASKAGLDHEGDSRIFKKIHDVVTKQIKVILRLIAVYAELVAIIG

SEQ ID NO: 118 - skipped

SEQ ID NO: 119 - protein, 484 aa, synthetic construct
  REGION 1..484: note = Synthetic
MGNTHFIVVHGGEEARQLAETAVREISKKEGTEVRFEKKDGLLSIEVKNLSEELQRLIQELLQLLVRLAALLEAVRAVEEAIKRNPDNDEAVETAVRLARELKKVAELLQELAKKAGVPAILRGALLALEVAVRAVELAIKSNPDNDEAVETAVRLARELLKVAAELAERAAKTKDEELIKLAQRAIEVAQRAVELAKKSNKDNEEAEKTRLALEAMELALQIFRQLKNTELAREAIELATEAAKATDSEALKVVKLALKIVQQLPDTELAKEALELAKEAVKSTDSEALKVVELALEIVQQLPDTELAKEALKLAKEAVKSTDSEALKVVYLALRIVQQLPDTELAREALELAKEAVKSTDSEQLEVVRLALEIVQLAPDTRLARAALKLAKEAVKSTDQEELKKVKAILRVASEVLKLEEEAKKSQEEVERLKQEVEKASKAGLDHEGDSRIFKKIHDVVTKQIKVILRLIAVYAELVAIIG

SEQ ID NO: 120 - protein, 305 aa, synthetic construct
  REGION 1..305: note = Synthetic
MGTTNFHLINGSEEARQIIEKIVEEVARKAGTEVHFEKSDGTLEIRVKNLHEELERRIKKAIEAALAAQIAKKLAEQIERQLSNTELAKEAKKLATEAEKARDSEAVQVVLLALEIVQQLPDTELAKEALELAEEAVKSTDSEALKVVKLALEIVQQLPDTELAREALELAKEAVKSTDSEALKVVYLALRIVQQLPDTELARLALELAKKAVEMTAQEVLEIARAALKAAQAFPNTELAELMLRLAEVAARVMKELERNDEEIKKSDELDDESLLEDIVELLKEIIKLWKILVEVSDVMLKLIS

SEQ ID NO: 121 - DNA, 21 bp, synthetic construct
  source 1..21: mol_type = other DNA
taagaaggagatatcatcatg

SEQ ID NO: 122 - DNA, 21 bp, synthetic construct
  source 1..21: mol_type = other DNA
taaagaaggagatatcatatg

SEQ ID NO: 123 - protein, 8 aa, synthetic construct
WSHPQFEK

SEQ ID NO: 124 - protein, 49 aa, synthetic construct
KAKEEVRELNEEFKEAEKRFRKLQEETKKALEEVEELNQRFEEALEEVE

SEQ ID NO: 125 - protein, 49 aa, synthetic construct
ERLKELREEWKRLRERLQELEEQHRELQEELKELREKWKRLKERLEELR

SEQ ID NO: 126 - protein, 7 aa, synthetic construct
EEKEKKR

SEQ ID NO: 127 - protein, 7 aa, synthetic construct
DEKREEE

SEQ ID NO: 128 - protein, 7 aa, synthetic construct
KEEERRQ

SEQ ID NO: 129 - protein, 7 aa, synthetic construct
VFFTVFV

SEQ ID NO: 130 - protein, 7 aa, synthetic construct
KNWQLNL

SEQ ID NO: 131 - protein, 7 aa, synthetic construct
RKRKEEE

SEQ ID NO: 132 - protein, 7 aa, synthetic construct
EEKEEQE

SEQ ID NO: 133 - protein, 7 aa, synthetic construct
EREEERE

SEQ ID NO: 134 - protein, 7 aa, synthetic construct
RERQEKR

SEQ ID NO: 135 - protein, 7 aa, synthetic construct
LLLLLLL

SEQ ID NO: 136 - protein, 7 aa, synthetic construct
LWLHLWL

SEQ ID NO: 137 - protein, 7 aa, synthetic construct
RKERQEG

SEQ ID NO: 138 - protein, 7 aa, synthetic construct
ELVHLVL

SEQ ID NO: 139 - protein, 7 aa, synthetic construct
LQIILQL

SEQ ID NO: 140 - protein, 7 aa, synthetic construct
ESREASA

SEQ ID NO: 141 - protein, 7 aa, synthetic construct
ALALALA

SEQ ID NO: 142 - protein, 7 aa, synthetic construct
EEREERE

SEQ ID NO: 143 - protein, 7 aa, synthetic construct
KEEKKEE

SEQ ID NO: 144 - protein, 7 aa, synthetic construct
EEEEEEE

SEQ ID NO: 145 - protein, 7 aa, synthetic construct
KKQRKKE

SEQ ID NO: 146 - protein, 7 aa, synthetic construct
RREQRKR

(19) United States
(12) Patent Application Publication
(10) Pub. No.: US 2024/0029824 A1
(43) Pub. Date: Jan. 25, 2024

(54) De novo design of obligate ABC heterotrimeric proteins

(71) Applicant: University of Washington, Seattle, WA (US)

(72) Inventors: David BAKER, Seattle, WA (US); Sherry BERMEO, Seattle, WA (US); Andrew FAVOR, Seattle, WA (US); Scott BOYKEN, Seattle, WA (US); Yang HSIA, Seattle, WA (US)

(21) Appl. No.: 18/310,680

(22) Filed: May 2, 2023

Related U.S. Application Data
(60) Provisional application No. 63/338,260, filed on May 4, 2022.

(51) Int. Cl. G16B 20/00; G16B 15/20

(52) U.S. Cl. CPC G16B 20/00; G16B 15/20

(57) ABSTRACT

Disclosed are polypeptides having an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of the amino acid sequences listed in Tables 1, 2, and 3, and heteropolymers formed from such polypeptides.

### CROSS REFERENCE

This application claims priority to U.S. Provisional Patent Application Ser. No. 63/338,260 filed May 4, 2022, incorporated by reference herein in its entirety.

### BACKGROUND

The de novo design of three protein chains that associate to form an obligate ‘ABC’ heterotrimer, but not binary AB, AC and BC heterodimers, is an outstanding challenge for protein design. ABC heterotrimers are a difficult design challenge because of the interaction cooperativity required for the three unique components to assemble only into the desired structure. Although an ABC heterotrimer only has one extra component compared to a heterodimer, the latter has only four alternate species, while an ABC heterotrimer has 15 alternate species.

### REFERENCE TO AN ELECTRONIC SEQUENCE LISTING

A computer readable form of the Sequence Listing is filed with this application by electronic submission and is hereby incorporated by reference in its entirety. The Sequence Listing is contained in the XML file created on Apr. 20, 2023 having the name “22-0585-US.xml” and is 155,987 bytes in size.

### SUMMARY OF THE DISCLOSURE

In one aspect, the disclosure provides polypeptide comprising an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of the amino acid sequences listed in Tables 1, 2, and 3, wherein 0-7 residues at the N and/or C-terminus are optional and may be absent and not considered when determining percent identity. In one embodiment, interface residues in the reference sequence as identified in Table 4 are maintained. In another embodiment, residues capable of hydrogen-bonding as identified in Table 4 are maintained. In a further embodiment, W, Y, and F residues in the reference sequence are maintained. In one embodiment, mutations in residues relative to the reference sequence are conservative amino acid substitutions.

In a further embodiment, the disclosure provides fusion proteins comprising (a) a polypeptide of any embodiment or combination of embodiments; (b) a second polypeptide; and (c) an optional amino acid linker linking the polypeptide and second polypeptide. In one embodiment, the second polypeptide comprises a helical repeat protein or a protein with mixed alpha helix/beta sheet secondary structure. In a further embodiment, the fusion protein comprises an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of sequences in Tables 5 and 6.

The disclosure also provides nucleic acids encoding a polypeptide or fusion protein of any embodiment herein; expression vectors comprising a nucleic acid of the disclosure operatively linked to a suitable control element, and host cells comprising the polypeptide, fusion protein, nucleic acid, and/or expression vector of any embodiment herein.

The disclosure further provides heterotrimers, heterodimers, or heterotetramers comprising polypeptides that comprise an amino acid sequence at least 70%, 75%, 80%, 85%, 90%, 91%, 92%, 93%, 94%, 95%, 96%, 97%, 98%, 99%, or 100% identical to an amino acid sequence combinations listed in Table 1, Table 2, Table 3, Table 5, or Table 6, wherein 0-7 residues at the N and/or C-terminus of the polypeptides are optional and may be absent and not considered when determining percent identity. In one embodiment, the heterotrimer, heterodimer, or heterotetramer comprises an interaction hub building block between chains in larger closed structures.

The disclosure also provides kits comprising one or more polypeptide, fusion protein, nucleic acid, expression vector, host cell, and/or heterotrimer of any embodiment herein, and methods for use of the polypeptide, fusion protein, nucleic acid, expression vector, host cell, heterotrimer, and/or kit of any embodiment herein.

### DESCRIPTION OF THE FIGURES

FIG. 1. Overview of design approach. (a) Base heterotrimer design: (I) for an ABC coiled coil, three helices are individually sampled across 8 parameters, as shown by their respective symbols. These backbones are then coupled to (II) MC HBNet to find hydrogen bond networks spanning all three helices. For the helical bundle approach, to break down the combinatorial explosion that arises when all 17 parameters are sampled simultaneously, a stepwise approach was taken to break apart the search problem in three steps. (1) First backbone sampling is carried out for the three inner helices and one outer helix, and MC HBNet is used to identify the subset of backbones that can host a network spanning all four helices. (2) A 5th helix is then sampled and required to be part of the subsequent MC HBNet search that includes this new helix and the three inner helices, followed by (3) a final iteration to add the 6th helix and a final MC HBNet search between this new helix and the three inner helices. (III) Backbones from both approaches can be optionally trimmed, packed with phenylalanine and other aliphatic residues at the core, and decorated with charged residues at the surface to enhance electrostatic interaction across the chains. (IV) Only the 6-helix bundles need short designed loops to form three helical hairpin units and make the final base heterotrimer. Two possible loop combinations are shown, with clockwise or counter-clockwise closure, and with loops all facing the same direction (solid lines) or loops at opposing termini ends (dashed lines). (b) Designed helical repeat (DHR) monomers can be rigidly joined to both coiled coil and helical bundle heterotrimers through single fusions, which can then be combined to make 4-arm and 3-arm heterotrimers, respectively. (c) Heterotrimer arms can be combined with other designed building blocks to create higher order nanostructures, such as A2B2 heterotetramers or A3B3C4/A4B4C4 hetero-oligomers.

FIG. 2. Experimental characterization of designed single helix heterotrimers. (a) Cross-sections across the coiled coil heterotrimer (DHT01) show core packing across the inner 5 heptads making up the shared ABC interface, with hydrogen bond networks and hydrophobic packing highlighted. (b) Design models of DHT01 and its two 4-arm fusions and shown in cartoon representation. (c) Superosem 200 chromatograms and SDS-PAGE gel (inset) show 3 chains eluting at a monodisperse peak, (d) SAXS profiles indicate good quality of fit (U) between the respective design models and experimental data collected, and (e) CD spectra shows designs are helical and (f) thermal melt indicates they are stable up to 95° C.

FIG. 3. Experimental characterization of designed helical hairpin heterotrimers. (a) Design models for heterotrimers are shown from a top-down view with a cartoon representation and with hydrogen bond networks in stick representation. (b) Monodisperse SEC traces and (c) Design models for DHT03 hierarchical 1, 2, and 3-arms are shown in a cartoon representation, followed by (d) SEC indicating the ABC heterotrimer is still present. Components for 2arm_A21/B21/C and subsequent 3-arm fusions were separately expressed and individually purified, mixed at a 1:1:1 ratio, and then annealed to reconstitute the ABC heterotrimer.

FIG. 4. Recursive design of higher order assemblies. (a) SAXS profiles indicate good quality of fit (χ) between the design model (line) and experimental scattering data (black dots) for four A2B2 tetramers: three Type-I tetramers (C2-DHT01-01, C2-DHT01-02, C2-DHT01-03), and one Type II tetramer (C2-DHT01-04). (b) nsEM 3D reconstructions and 2D class averages for a Type I A2B2 heterotetramer (C2-DHT01-03, top), and a Type II A2B2 heterotetramer (C2-DHT01-04, bottom). (c) Characterization of C3-symmetric oligomers; nsEM 3D reconstruction and 2D class averages for two A3B3C3 cyclic designs, C3-DHT03-01 (top) and C3-DHT03-02 (bottom). (d) Characterization of C4-symmetric oligomers; nsEM 3D reconstruction and 2D class averages for two A4B4C4 cyclic designs, C4-DHT03-01 (top) and C4-DHT03-02 (bottom). (e) Fusion-accessibility of the a third chain enables further design opportunities; nsEM 3D reconstruction and 2D class averages for versions of C3-DHT03-01 and C4-DHT03-01 with DHR82 fused to chain C of DHT03 are shown.

FIG. 5. Helical wheel for coiled coil DHT01 and mutations at position ‘g’ affect ABC assembly. (a) Shared interfaces of all three chains of the heterotrimer are broken up by heptads and helical wheel positions, with the amino acid letters in bold indicating the residues found by Monte Carlo HBNet. Residues contributing to the designed hydrogen bond networks are found at ‘a,’ ‘d,’ ‘e,’ and ‘g’ positions (top to bottom: SEQ ID NO: 1, 124, and 125). (b) A helical wheel was drawn for each chain and then combined to form a heterotrimer wheel, with positions coded to indicate the presence of nonpolar, polar, or mixed (nonpolar and polar) residues. Letters in bold again match the residues found by Monte Carlo HBNet (top left to right: SEQ ID NOs: 126-136; bottom left to right: SEQ ID NOs: 137-146. (c) A helical wheel for a heterotrimer variant that now has only polar residues at position ‘g’ for chains A and B, with respective mutations shown. Experimental validation of this design by LC-MS indicates the absence of chain B in the IMAC pull-down eluate. (d) Three mutations of the variant heterotrimer from (c) are shown in cartoon and stick representation. It was hypothesized that polar or charged amino acids could help contribute to ionic interactions. One example of an ionic interaction designed for this peptide is shown to the lower right, as determined by a 1.8 Å x-ray crystal structure.

FIG. 6. CD plots for three helical bundle heterotrimers. (a) Wavelength plots indicate that all three heterotrimers are helical at 25° C. and can retain their secondary structure even after increasing the temperature to 95° C. and returning to 25° C. again. (b) Temperature plots indicate that all three heterotrimers are thermostable and do not completely unfold at 95° C.

FIG. 7. DHT03 can undergo different loop connectivities to form alternate desired oligomers. Different loop closures were applied to DHT03 to create a total of five heterodimers, one alternate ABC heterotrimer, and one ABCD heterotetramer. The alternate heterotrimer and ABCD heterotetramer were made by removing the existing loop on the chain C helical hairpin, resulting in single helices for each chain. Black solid lines represent loops facing all on the same side, while dashed lines represent loops on opposite ends of termini.

FIG. 8. Hierarchical building with DHT03 heterotrimer base. (a) Three more 2-arm constructs built off of 1arm_A21/B/C are shown in a cartoon representation. Curved and straight DHRs were used as input for rigid helical fusion. (b) These constructs had monodisperse peaks by SEC.

FIG. 9. Hierarchical building with DHT05 heterotrimer base. (a) Hierarchical building approach of DHT05 heterotrimer base is shown, with two successful 1-arm fusions, as determined by monodisperse SEC peaks and SAXS (dots, experimental data; line calculated from design model). (b) The one arm construct with a rigid fusion to chain A can be built into a 2-arm construct with another rigid fusion to chain B, as determined by SEC and SAXS. (c) The two 1-arm constructs from (a) can be combined and sustain another rigid fusion to chain B to create a 3-arm construct, as determined by SEC and SAXS indicating only the formation of an ABC heterotrimer. (d) A 2-arm construct can be aligned to half of an LHD heterodimer fusion (LHD101B14)⁶ with compatible DHR and termini, and then cut and stitched together. This results in an extended 2-arm heterotrimer with one arm capable of binding the respective LHD heterodimer fusion partner, as determined by a leftward shift in the main SEC peak when LHD101A53 is mixed with the co-expressed 2-arm construct. This results in a four component ABCD heterotetramer. Determined radius of gyration via SAXS (Table 7) also matches the heterotetramer better compared to that of the heterotrimer above.

FIG. 10. Building block connections during cyclic fusion of A2B2 tetramers. (a) The cyclic design strategy for A2B2 tetramers involved splicing of two types of chain combinations from DHT01-4arm-02. For type-I A2B2 tetramers (top), the C-terminal DHR-arm of chain A is fused to the N-terminus of a different DHR repeat segment, which then has its C-terminus fused to the N-terminal DHR-arm of chain B. For type-II A2B2 tetramers (bottom), the C-terminal DHR-arm of chain A is fused to the N-terminus of a different DHR repeat segment, which then has its C-terminus fused to the N-terminal DHR arm of chain C. (b) The cyclic design strategy for ring-proteins made from the DHT03 helical bundle heterotrimer. Both DHR-arms in DHT03_2arm_A21/B21/C have only N-termini accessible for fusion, necessitating two separate fusion steps to form the cyclic structures, with each fusion step joining one of DHT03's DHR-extended arms to a C-terminal DHR chain of an LHD heterodimer, which possessed fusion-accessible C-termini on both of its chains.

FIG. 11. Computational protocol for realignment of PDB models to experimentally observed symmetry states. Several ring-proteins assembled with lower degrees of cyclic symmetry than their design models, as shown by nsEM; C3-DHT03-01 had originally been designed as a C4 ring, while C4-DHT03-01 and C4-DHT03-02 had been designed as C5 rings. Symmetry-correction in the resolved structures followed a 5-step protocol: (a) AlphaFold2⁸ was run for each chain (chains A and B) in the ring structure that had undergone WORMS fusion. For each chain, five candidate pdbs were generated, corresponding to predictions performed by AlphaFold2's five different sets of model parameters. (b) Predicted chain structures with high pLDDT were selected, two types of interface-aligned models were created by either aligning the predicted chain models to the DHT03 heterotrimer interface (top) or the LHD heterodimer interface (bottom). (c) Several duplicate copies (one copy per degree of symmetry) of the interface-aligned models containing both chains A and B were fit to the reconstructed 3D electronic density maps in ChimeraX™ in order to find reasonable starting positions for further refinement. For the DHT-aligned models, gaps were present at the LHD interface, and for the LHD-aligned models, gaps were present at the DHT interface. (d) Iterative cycles of alignment of between DHT-aligned chains to their LHD-aligned counterparts were performed, which gradually reduced the ring-closure error at both interfaces. (e) In order to refine the structure to satisfy energetics of proper interface formation, and to further optimize the structure's fit within the 3D reconstructed map, Rosetta relax was performed with an additional constraint¹⁰.

FIG. 12. Opportunities and limitations of designs using fusion of multiple distinct oligomers. (a) Oligomeric proteins provide scaffolds upon which functional motifs can be affixed, with a common application being the fusion of oligomers to protein domains that bind targets of interest. When using cyclic homo-oligomers, a central benefit lies in their presentation of motifs with high valency, which increases with the oligomer's degree of symmetry. On the other hand, heterodimers provide an ability to present two distinct functional motifs in conjunction. Heterotrimers extend such possibilities by enabling the presentation of three distinct motifs at a time. (b) Heterotrimers allow for the combined benefits of homo-oligomers and heterodimers in motif presentation, by allowing two distinct motifs to be spliced to two of their three chains, while joining the third chain to a symmetric homo-oligomer, allowing an increase in valency while presenting multiple distinct motifs in conjunction. Additionally, the number of distinct motifs that can be presented by these constructs can be increased further by fusing multiple heterotrimers to a central heterodimer, or another central heterotrimer. (c) When fusions are made using two homo-oligomers, possessing a specific degree of cyclic symmetry about their interfaces, the higher-order geometries of such designs are limited to those possessing the same point-group symmetries as their constituent building blocks. Specifically, the classes of higher order symmetry resulting from fusion of two symmetric homo-oligomers are either the regular polyhedral groups (nanocages) or wallpaper groups (2D lattices), as defined by the point-symmetries of their constituent interfaces. When using heterodimers to create higher order assemblies through rigid fusion, two fusion-accessible termini are used up with each splice step, limiting the range of designs that can be made from such building blocks to simple ring structures, or chains of heterodimers that maintain only two fusion-accessible termini at splice steps. In contrast, higher ordered assemblies made through fusion of two or more heterotrimers are not limited to the geometric constraints of homo-oligomers or heterodimers; each rigid fusion step leaves a net increase in the number of fusion-accessible chains, enabling extensive opportunities for subsequent splicing. (d) If many distinct heterotrimers are available, it becomes possible to explore many new types of asymmetric constructs that cannot be designed using heterodimers or homo-oligomers. While heterotrimers can certainly be combined with homo-oligomers to create symmetric designs with additional branching, the design of extensively branching asymmetric designs is an avenue that can only be explored using heterotrimeric building blocks.

FIG. 13. Heterotrimers enable modular functionalization of higher order assemblies. (a) Ring proteins. When using heterodimers to create multimeric complexes with cyclic symmetry, the fusion-accessible termini from each subunit are both used up to form the splice junction. Using a heterotrimeric complex to form the interface between ring subunits uses only two chains for ring-closure, leaving the third chain available for fusion to additional scaffolds. (b) Nanocages. Homo-oligomers with cyclic symmetries can be rigidly fused to heterodimers, to form an interface connecting the symmetric centers in protein nanocages, while keeping the point-group interfaces on separate chains within two-component assemblies. However, in these constructs, all fusion-accessible termini are lost by junction formation, which limits the ability to affix further functional motifs or antigens to these structures. In contrast, when using two chains from a heterotrimer to form the interface between the chains forming symmetric interfaces, the third chain remains available for splicing. The fusion-accessibility of the third chain provides extensive design opportunities; for a given nanocage containing a heterotrimeric interface, the third chain can be fused to any number of motifs or antigen-presenting domains, providing many functional opportunities for cage customization without redesigning the base cage scaffold itself. (c) Unbounded assemblies. Two-dimensional lattices can be formed by bridging symmetric point-group interfaces. In these designs, protein chains that form cyclic-symmetric interfaces are connected to chains that form either C2 or D2 symmetric interfaces; interfaces connecting the symmetric centers can be formed by fusion to the chains of a hetero-oligomer. When using heterodimeric interfaces to connect the two types of symmetry-forming chains, a 2-component sheet can be constructed; fusion-accessible termini are used up during splicing, leaving limited opportunities to array functional motifs upon these lattices. When using two chains from a heterotrimer to join the two symmetry-forming components, the third chain is again left accessible for further design opportunities. (d) In the case of sheets containing dihedral interfaces, the cyclic symmetric centers periodically alternate orientation, flipping about the plane of the sheet. When heterotrimers are incorporated into this system, the third chain is available for fusion to an additional C2-symmetric component, forming interfaces in the direction normal to the sheet. Through this arrangement, lattices joined together by heterotrimers can assemble layered sheets, enabling the design of anisotropic biomaterials. (e) In sheets possessing C2 interfaces, asymmetry between the two sides of the sheet is maintained, providing opportunities for one-directional display of functional motifs or antigens.

Through the ability to array functional domains in a controlled, periodic manner on one surface of a 2D-lattice, such designs can be utilized for a wide range of applications within protein-based nanotechnology.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2^{nd} Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Any N-terminal methionine residue may be present in the polypeptides of the disclosure, or may be deleted and not considered when determining percent identity.

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

In a first aspect, the disclosure provides polypeptides comprising an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of the amino acid sequences listed in Tables 1, 2, and 3 (SEQ ID NO:1-43, 47, 55, 59, 65, 88, 89, 95, and 98), wherein 0-7 residues at the N and/or C-terminus are optional and may be absent and not considered when determining percent identity.

As disclosed by the inventors in the examples, the polypeptides are capable of associating with other protein chains to form an obligate ‘ABC’ heterotrimers (Table 1), but not binary AB, AC and BC heterodimers when all three chains are present; obligate heterodimers (Table 2), or obligate heterotetramers (Table 4). The polypeptides can be used to create cooperative heteropolymers that can assemble as freestanding helical units or as hubs in larger designed assemblies. Their small base size and high soluble expression make them useful, for example, in biological scaffolding applications involving the recruitment or display of three different proteins. Fusion of polypeptide chains to other proteins for scaffolding applications may be covalent, or simply through flexible linkers sequences.

The polypeptides have the ability to sustain rigid helical fusions to monomeric repeat proteins (as described for the fusion proteins of the disclosure), enabling the incorporation of arms that can be extended to provide three new and unique elongated connection points. The heteropolymers can be used as interaction hub building blocks between chains in larger closed structures generated through geometry-aware rigid fusion to themselves and other designed proteins. A notable feature is that these new assemblies can continue to be built out recursively, unlike previously designed rings, as they have outward facing chains with two or more free termini. The number and orientational accessibility of the chain termini found in the heteropolymers enable the display of multiple distinct functional domains for signaling, recruitment and engagement of specific cells, the study of protein-protein interactions, and other applications. As illustrated by the larger ring designs, the modularity and orthogonality of the designed protein interfaces makes it possible to combine the heterotrimers with other heteromeric building blocks to construct more diverse nanostructures.

As used herein, “wherein 0-7 residues at the N and/or C-terminus are optional and may be absent” means that the N-terminal 1, 2, 3, 4, 5, 6, or 7 amino acid residues may be absent, and/or the C-terminal 7, 6, 5, 4, 3, 2, or 1 amino acid residues may be absent, and are not considered when determining percent identity.

TABLE 1
Obligate Heterotrimers

| | | | | Seq ID |
|---|---|---|---|---|
| Name | State | Chain | Design sequences | combos |
| DHT01 | ABC | A | DEEEKLRESLEEQKKRVKEIEREHEEIREALKRLQESVKRQEEA | 1 + 2 + 3 |
| | | | LRQLG (SEQ ID NO: 1) | |
| | | B | SEQERREKAKEEVRELNEEFKEAEKRFRKLQEETKKALEEVEEL | |
| | | | NQRFEEALEEVERIKRRQK (SEQ ID NO: 2) | |
| | | C | DEEEEKKKEEKKKEERLKELREEWKRLRERLQELEEQHRELQEE | |
| | | | LKELREKWKRLKERLEELREKLRREEQRRREEK (SEQ ID | |
| | | | NO: 3) | |
| DHT02 | ABC | A | PDSDELRKLLKQLEDLLQQLKDLIDKAERHNGDNDVILELHEIY | 4 + 5 + 6 |
| | | | LRLLDIYQKLAKIILKLLR (SEQ ID NO: 4) | |
| | | B | PQLDKFRDESRYLDKASKEVEDEIKKIEKIIRDADEKSLPEEAL | |
| | | | QDVVKSVVKVYLKSVKETIKSIELVIELIKVIM (SEQ ID | |
| | | | NO: 5) | |
| | | C | ETEEIAYSSREAEKELKKIEEEQKKAKKELEEQKRQNGEPSSEI | |
| | | | SKQVLKLILKALKVIAKIQKSAQRQQDALIKILS (SEQ ID | |
| | | | NO: 6) | |
| DHT03 | ABC | A | PEEEKLKELLKELKKVLDRLKKILERNDEEIKKSDELDDESLLE | 7 + 8 + 9, |
| | | | DIVELLKEIIKLWKILVELSDILLKLIS (SEQ ID NO: 7) | or |
| | | B | PVDEIDKEVKKLEEEAKKSQEEVERLKQEVEKASKAGLDHEGDS | 7 + 8 + 59, |
| | | | RIFKKIHDVVTKQIKVIIRLIEVYVRLVEIIL (SEQ ID | or |
| | | | NO: 8) or | |
| | | | PVDEIDKEVKKLEEEAKKSQEEVERLKQEVEKASKAGLDHEGDS | 7 + 55 + 65, |
| | | | RIFKKIHDVVTKQIKVILRLIAVYAELVAIIG (SEQ ID | or |
| | | | NO: 55) | |
| | | C | KQKEAIKVYLELLEVHSRVLKALIEQIKLFIELIKRPDEDLADK | 7 + 55 + 9, |
| | | | VRKSSEELKKIIKEVEKILRKVDDILYKVKS (SEQ ID | or |
| | | | NO: 9) or | |
| | | | KQKEAIKVYLELLEVHSRVLKALIEQIKLFIELIMEPDEDLADK | 7 + 55 + 59, |
| | | | VRKSSEELKKIIKEVEKILRKVDDILEKVKS (SEQ ID | or |
| | | | NO: 59) or | |
| | | | KQKEAIKVYLELLEVHSRVLKALIEQILLFIELIVRPDEDLADK | 7 + 8 + 65 |
| | | | VRKSSEELKKIIKEVEKILRKVDDILYKVKS (SEQ ID | |
| | | | NO: 65) | |
| DHT04 | ABC | A | PERELLELLIRLFNSQVKLFELQKELLELLRELLEENSEDNSRK | 10 + 11 + 12 |
| | | | LEDLLRKIQELLKENQDLLYTVEDVLRKLK (SEQ ID | |
| | | | NO: 10) | |
| | | B | DEELIIYIIELLLKDDELLLKSLEIILRIIQLQLTSKDKDDGTD | |
| | | | KIKKLVDESKKILDDSERLAEELRKIEEKLK (SEQ ID | |
| | | | NO: 11) | |
| | | C | PKEEIRKLLKLLLRLNELLLKLHELLLRLLTNQGDHETTDEVVK | |
| | | | RYDKIVKDYDKIVKDIKEIIDKLL (SEQ ID NO: 12) | |
| DHT05 | ABC | A | PEEKKEIKRTTEEVKEEFRKIQEEIKKLEEEAKKAEKGNGKEEI | 13 + 14 + 15, |
| | | | KELLLRLSELLARSLQLLAQQIEAIAKLIRG (SEQ ID | or |
| | | | NO: 13) | |
| | | B | SEVEKRLLEIHRRVAESHRILVEVHEALIEALRGNSEEAKEKLK | 13 + 14 + 89, |
| | | | ELVKKLEKIIKEEEELLKKLEKIVKEAS (SEQ ID NO: 14) | or |
| | | | or | |
| | | | SEVEKRLLEIHRRVAESHRILVEVHEALIEALRGNSEEAKEKLK | 13 + 88 + 95, |
| | | | ELVKELEKIIKEEEELLKELEKIVEEAS (SEQ ID NO: 88) | or |
| | | C | PDEEELEKLLRKIRELIKEIEEVIKEYQELKERGDGGEKEEKEL | 13 + 88 + 15 |
| | | | IQANLRLLKLHTRLLKLYLELLKLIIKQG (SEQ ID NO: 15) | |
| | | | or | |
| | | | PDEEELEKLLRKIRELIKEIEEVIKEYQKLKERGDGGEKEEKEL | 13 + 88 + 89, |
| | | | IQANLRLLKLHTRLLKLYLELLKLIIKQG (SEQ ID NO: 89) | or |
| | | | or | |
| | | | PDEEELEKLLRKIRELIKEIEEVIKEYQELKERGDGGEKEEEEL | 13 + 14 + 95, |
| | | | IQANLRLLKLHTRLLKLYLELLKLIIKQG (SEQ ID NO: 95) | or |
| | | | or | |
| | | | PDEEELEKLLRKIRELIKEIEEVIKEYQELKERGDGGEKEEKEL | 13 + 14 + 98, |
| | | | IQANLRLLKLHTELLKLYLELLKLIIKQG (SEQ ID NO: 98) | or |
| | | | | 13 + 88 + 98 |
| DHT06 | ABC | A | DEKELAKTNAEILKTLLELFEAESRLQEIILKLLERPDEKEEDE | 16 + 17 + 18 |
| | | | LKRALERLKEALERLEEAIKRYEETLKRLL (SEQ ID | |
| | | | NO: 16) | |
| | | B | SEEDELEKAIREQKKALERLKEAIERYEETLKRGKNEKELAKTN | |
| | | | AEILKTLLELFKSQLELAEIILKLLR (SEQ ID NO: 17) | |
| | | C | SEEDELRKLTEYQNRALEELEKAIKEYEKTLRRLLGAPDERDEK | |
| | | | ELAETNARILRTLLELFRTASEALDIILKLLR (SEQ ID | |
| | | | NO: 18) | |
| DHT07 | ABC | A | SEKELIQLYVEAQELYAEAFELQVEIFKLLVKILSANSPKDEKE | 19 + 20 + 21 |
| | | | KIEKRIEEVQKRIKEIQERLQEIQKRLEEAG (SEQ ID | |
| | | | NO: 19) | |
| | | B | DREEKLERETEKLEKKIRESQRRMEELRKQIETDPDEKELLEIL | |
| | | | IRSLRLLEEQLEALRKLNHAAQQLLG (SEQ ID NO: 20) | |
| | | C | SEREVIQRLIEVSEELSRATERLVEAHKIVLRAIASPNESEKEE | |
| | | | IQKLAKELEEILKKLKKVIKESRELAEKAS (SEQ ID | |
| | | | NO: 21) | |
| DHT08 | ABC | A | SKEEIEKELKKLKKELEELKREIEKSTKKLEEVRGSNSEKELIQ | 22 + 23 + 24 |
| | | | IIKEQIEISERLLEISTRLLEIIIKLLG (SEQ ID NO: 22) | |
| | | B | DKEEKIKEILRRVQELRREHEELEERLKELKKELEKSPSQEELK | |
| | | | KFIKLAQELIKLTLELIELSLRLIELLLG (SEQ ID NO: 23) | |
| | | C | DEEEVKEVAKEYREEVRKFKETIKEIRERIKRGSNEKEMIKLYV | |
| | | | KLIELHTRLVKLHTKIVEILVKLLL (SEQ ID NO: 24) | |
| DHT09 | ABC | A | SEDEDLIRSVRVIQKSVEIILEMVKIIQELLKSTSEEELKELLK | 25 + 26 + 27 |
| | | | KLKRLIEKLEELLRQYEELLKKIKG (SEQ ID NO: 25) | |
| | | B | SKEEILKLHLEVAEIHIETVKIYLEVIKILIRLI EKNSSKEDER | |
| | | | ELEEELKELERELEELQKKLEEIRKELQEIEG (SEQ ID | |
| | | | NO: 26) | |
| | | C | SREETIIQFTKLLIKFNSLLVKHLKLIIRFIELEENNSSKESEK | |
| | | | EILEESKRIVERLEEIVRELQKLSKRLEEIIG (SEQ ID | |
| | | | NO: 27) | |
| DHT10 | ABC | A | SEEEELKKLQEEIKKLKKQVERLEKESKELEKRLEKSPSEEIAK | 28 + 29 + 30 |
| | | | ELLRITAELVEISEELAEATKRLLEIAG (SEQ ID NO: 28) | |
| | | B | SELEEEIKRIRKEIEESRRRIEELRKKAQKAREGSSEKEFLQIY | |
| | | | IELQKVLIEQIKIYIELLQIIFELQS (SEQ ID NO: 29) | |
| | | C | SEKEEAEKKAREVKKRIEEQEKESKKVKEEAKKLGPDTEVLKIL | |
| | | | IEVHELHIESLRVVAEITQALVEILK (SEQ ID NO: 30) | |
| DHT03_ | ABC | A | HEELADEVRKSSEELKKIIEEVEKILRKVDDILYKVG (SEQ | 41 + 42 + 43 |
| variant_ | | | ID NO: 41) | |
| ABC-2 | | | | |
| | | B | QKEAIKVYLELLEVHSRVLKALIEQIKLFIELIKRK (SEQ ID | |
| | | | NO: 42) | |
| | | C | PDEIDKEVKKLEEEAKKSQEEVERLKQEVEEASKAGLDHEADSR | |
| | | | IFKKIHDVVTKQIKVIIRLIEVYVRLAEIIGEPSEKLKELLKEL | |
| | | | KKVLDRLKKILERNDEEIKKSDELDDESLLEDIVELLKEIIKLW | |
| | | | KILVELSDILLKLID (SEQ ID NO: 43) | |

TABLE 2
Obligate Heterodimers

| | | | | SEQ ID |
|---|---|---|---|---|
| name | State | Chain | Sequence | Combo |
| DHT03_ | AB | A | DESLLEDIVELLKEIIKLWKILVELSDILLKLIGGDD | 31 + 32 |
| variant_ | | | KVRKSSEELKKIIKEVEKILRKVDDILYKVSAPSEEK | |
| AB-1 | | | QKEAIKVYLELLEVHSRVLKALIEQIKLFIELIKR | |
| | | | (SEQ ID NO: 31) | |
| | | B | PDEIDKEVKKLEEEAKKSQEEVERLKQEVEEASKAGL | |
| | | | DHEADSRIFKKIHDVVTKQIKVIIRLIEVYVRLVEII | |
| | | | LGSGSDEKLKELLKELKKVLDRLKKILERNDEEIKKS | |
| | | | (SEQ ID NO: 32) | |
| DHT03_ | AB | A | DHEADSRIFKKIHDVVTKQIKVIIRLIEVYVRLVEII | 33 + 34 |
| variant_ | | | LGSGSDEKLKELLKELKKVLDRLKKILERNDEEIKKS | |
| AB-2 | | | DELDDESLLEDIVELLKEIIKLWKILVELSDILLKLI | |
| | | | D (SEQ ID NO: 33) | |
| | | B | HEELADKVRKSSEELKKIIKEVEKILRKVDDILEKVS | |
| | | | APSEEKQKEAIKVYLELLEVHSRVLKALIEQIKLFIE | |
| | | | LIKRKVKRVPDEIDKEVKKLEEEAKKSQEEVERLKQE | |
| | | | VEEASKAG (SEQ ID NO: 34) | |
| DHT03_ | AB | A | DESLLEDIVELLKEIIKLWKILVELSDILLKLIGGDD | 35 + 36 |
| variant_ | | | KVRKSSEELKKIIKEVEKILRKVDDILYKVSAPSEEK | |
| AB-3 | | | QKEAIKVYLELLEVHSRVLKALIEQIKLFIELIKRKH | |
| | | | KRVPDEIDKEVKKLEEEAKKSQEEVERLKQEVEEASK | |
| | | | AG (SEQ ID NO: 35) | |
| | | B | DHEADSRIFKKIHDVVTKQIKVIIRLIEVYVRLVEII | |
| | | | LGSGSDEKLKELLKELKKVLDRLKKILERNDEEIKKS | |
| | | | (SEQ ID NO: 36) | |
| DHT03_ | AB | A | DHEADSRIFKKIHDVVTKQIKVIIRLIEVYVRLVEII | 37 + 38 |
| variant_ | | | LGSGSDEKLKELLKELKKVLDRLKKILERNDEEIKKS | |
| AB-4 | | | DELDDESLLEDIVELLKEIIKLWKILVELSDILLKLI | |
| | | | GGDDKVRKSSEELKKIIKEVEKILRKVDDILYKVS | |
| | | | (SEQ ID NO: 37) | |
| | | B | QKEAIKVYLELLEVHSRVLKALIEQIKLFIELIKRKH | |
| | | | KRVPDEIDKEVKKLEEEAKKSQEEVERLKQEVEEASK | |
| | | | AG (SEQ ID NO: 38) | |
| DHT03_ | AB | A | QKEAIKVYLELLEVHSRVLKALIEQIKLFIELIK | 39 + 40, |
| variant_ | | | (SEQ ID NO: 39) | or 42 + 40 |
| AB-5 | | | or | |
| | | | QKEAIKVYLELLEVHSRVLKALIEQIKLFIELIKRK | |
| | | | (SEQ ID NO: 42) | |
| | | B | PVDEIDKEVKKLEEEAKKSQEEVERLKQEVEKASKAG | |
| | | | LDHEGDSRIFKKIHDVVTKQIKVIIRLIEVYVRLVEI | |
| | | | ILGSGSDEKLKELLKELKKVLDRLKKILERNDEEIKK | |
| | | | SDELDDESLLEDIVELLKEIIKLWKILVELSDILLKL | |
| | | | IDRDTHEDLADKVRKSSEELKKIIKEVEKILRKVDDI | |
| | | | LYKVKS (SEQ ID NO: 40) | |

TABLE 3
Obligate heterotetramer

| | | | | SEQ ID |
|---|---|---|---|---|
| Name | State | Chain | Sequence | Combo |
| DHT03_ | ABCD | D | DEDLADKVRKSSEELKKIIKEVEKILRKVDDILYKVKS | 7 + 8 + 39 + |
| variant_ | | | (SEQ ID NO: 47) | 47 |
| ABCD-1 | | | | |

In one embodiment, interface residues in the reference sequence as identified in Table 4 are maintained in the polypeptides of the disclosure. The interface residues are those residues at the interface of a heteropolymer that includes the polypeptide. Table 4 shows the position of interface residues relative to the amino acid numbering of sequences shown in Tables 1-3. By way of non-limiting example, interface residues for SEQ ID NO:1 (DHT01 chain A) are residues 9, 10, 13, 17, 20, 24, 27, 30, 31, 34, 37, 38, and 41. Thus, in one embodiment, residues 9, 10, 13, 17, 20, 24, 27, 30, 31, 34, 37, 38, and 41 in the polypeptide are identical to those in SEQ ID NO:1. It will be apparent to those of skill in the art to determine the interface residues in the other sequences listed in Table 4.

As used herein, “maintained” means identical amino acid residue as in the reference protein.

In another embodiment, residues capable of hydrogen-bonding as identified in Table 4 are maintained in the polypeptides of the disclosure. Table 4 shows the position of hydrogen-bonding residues (“HBNet residues”) relative to the amino acid numbering of sequences shown in Tables 1-3. By way of non-limiting example, hydrogen-bonding residues for SEQ ID NO:1 (DHT01 chain A) are residues 9, 13, 24, 37, 41. Thus, in one embodiment, residues 9, 13, 24, 37, and 41 in the polypeptide are identical to those in SEQ ID NO:1. It will be apparent to those of skill in the art to determine the hydrogen-bonding residues in the other sequences listed in Table 4.

In a further embodiment W, Y, and F residues in the reference sequence are maintained in the polypeptides of the disclosure. Table 4 identifies positions of W, Y, and F (large aromatic) residues in the reference sequences. By way of non-limiting example, large aromatic residues for SEQ ID NO:2 (DHT01 chain B) are residues 20, 27, and 48. Thus, in one embodiment, residues 20, 27, and 48 in the polypeptide are identical to those in SEQ ID NO:2. It will be apparent to those of skill in the art to determine the large aromatic residues in the other sequences listed in Table 4.

TABLE 4

| | | | | Large | |
|---|---|---|---|---|---|
| | | | | aromatic | |
| | | | | residues | |
| Design | | | HBNet | (TRP, TYR, | Loop |
| name | Chain | Interface residues | residues | PHE) | residues |
| DHT01 | A (SEQ | 9, 10, 13, 17, 20, 24, | 9, 13, | n/a | n/a |
| | ID | 27, 30, 31, 34, 37, 38, | 24, 37, | | |
| | NO: 1) | 41 | 41 | | |
| | B (SEQ | 9, 13, 16, 17, 20, 24, | 17, 34, | 20, 27, 48 | n/a |
| | ID | 27, 30, 31, 34, 37, 38, | 45 | | |
| | NO: 2) | 41, 44, 45, 48, 51, 52, | | | |
| | | 55 | | | |
| | C (SEQ | 17, 20, 24, 27, 31, 34, | 24, 37, | 24, 52 | n/a |
| | ID | 37, 38, 41, 42, 45, 48, | 38, 42, | | |
| | NO: 3) | 52, 55, 59, 62, 66 | 52 | | |
| DHT02 | A (SEQ | 9, 10, 13, 16, 17, 20, | 41, 44, | 44, 51 | 31-33 |
| | ID | 23, 24, 27, 34, 36, 37, | 51, 52 | | |
| | NO : 4) | 38, 40, 41, 44, 45, 47, | | | |
| | | 48, 50, 51, 52, 54, 55 | | | |
| | B | 10, 13, 16, 17, 20, 24, | 10, 17, | 55 | 37-40 |
| | (SEQ ID | 27, 30, 31, 34, 44, 45, | 55, 58, | | |
| | NO : 5) | 47, 48, 51, 52, 54, 55, | 62, 65 | | |
| | | 56, 58, 59, 62, 63, 65, | | | |
| | | 66, 68, 69, 70 | | | |
| | C | 9, 12, 16, 19, 23, 26, | 9, 23, | n/a | 38-41 |
| | (SEQ ID | 30, 34, 44, 45, 48, 49, | 63, 67, | | |
| | NO : 6) | 51, 55, 56, 58, 59, 60, | 69, 70 | | |
| | | 62, 63, 65, 66, 67, 69, | | | |
| | | 70 | | | |
| DHT03 | A (SEQ | 9, 10, 13, 16, 17, 20, | 27, 57, | 57 | 34-38 |
| | ID | 23, 24, 27, 31, 42, 43, | 64 | | |
| | NO : 7) | 46, 47, 49, 50, 53, 54, | | | |
| | | 56, 57, 59, 60, 61, 63, | | | |
| | | 64 | | | |
| | B | 9, 12, 16, 19, 23, 26, | 19, 51, | 47, 68 | 36-39 |
| | (SEQ ID | 30, 33, 44, 46, 47, 50, | | | |
| | NO: 8 or | 51, 53, 54, 55, 57, 58, | 55, 57, | | |
| | 55) | 60, 61, 64, 65, 67, 68 | 68 | | |
| | C | 8, 9, 10, 12, 13, 15, | 9, 16, | 9, 30 | 36-38 |
| | (SEQ ID | 16, 17, 19, 20, 22, 23, | | | |
| | NO : 9, | 24, 26, 27, 29, 30, 31, | 17, 26, | | |
| | 59, or | 33, 34, 41, 42, 45, 48, | 48, 49 | | |
| | 65) | 49, 52, 55, 56, 59, 62, | | | |
| | | 63, 66 | | | |
| DHT04 | A | 8, 9, 10, 12, 13, 15, | 15, 16, | 13, 20 | 36-38 |
| | (SEQ ID | 16, 17, 19, 20, 22, 23, | 23, 59 | | |
| | NO: 10) | 26, 27, 29, 30, 33, 34, | | | |
| | | 45, 48, 49, 52, 55, 56, | | | |
| | | 59, 62, 63, 66 | | | |
| | B (SEQ | 8, 9, 11, 12, 13, 15, | 15, 16, | n/a | 36-38 |
| | ID | 16, 18, 19, 20, 22, 23, | 22, 53, | | |
| | NO: 11) | 25, 26, 27, 29, 30, 32, | 60 | | |
| | | 34, 46, 49, 50, 53, 56, | | | |
| | | 57, 60, 63, 64, 67 | | | |
| | C (SEQ | 8, 9, 11, 12, 13, 15, | 16, 23, | 46, 53 | 32-36 |
| | ID | 16, 18, 19, 20, 22, 23, | | | |
| | NO: 12) | 25, 26, 27, 29, 30, 31, | 46, 53 | | |
| | | 39, 42, 43, 46, 49, 50, | | | |
| | | 53, 56, 57, 60 | | | |
| DHT05 | A (SEQ | 10, 11, 14, 18, 21, 25, | 10, 11, | 18 | 38-40 |
| | ID | 28, 32, 35, 44, 47, 48, | 52, 58, | | |
| | NO: 13) | 49, 51, 52, 54, 55, 56, | 65 | | |
| | | 58, 59, 61, 62, 63, 65, | | | |
| | | 66, 68 | | | |
| | B | 8, 10, 11, 14, 15, 17, | 11, 17, | n/a | 34-36 |
| | (SEQ ID | 18, 20, 21, 22, 24, 25, | 18, 25, | | |
| | NO: 14 | 27, 28, 29, 31, 32, 39, | 57 | | |
| | or 88) | 43, 46, 47, 50, 53, 54, | | | |
| | | 57, 60, 61, 64 | | | |
| | C | 9, 10, 13, 16, 17, 20, | 27, 48, | 27, 62 | 34-37 |
| | (SEQ ID | 23, 24, 27, 30, 44, 45, | 55, 56, | | |
| | NO: 15, | 47, 48, 49, 51, 52, 54, | 62 | | |
| | 89, 95, | 55, 56, 58, 59, 61, 62, | | | |
| | or 98) | 63, 65, 66 | | | |
| DHT06 | A (SEQ | 8, 9, 10, 12, 13, 15, | 9, 23, | 20, 66 | 36-38 |
| | ID | 16, 17, 19, 20, 22, 23, | 24, 66 | | |
| | NO: 16) | 24, 26, 27, 29, 30, 31, | | | |
| | | 33, 34, 45, 48, 49, 52, | | | |
| | | 55, 56, 59, 62, 63, 66 | | | |
| | B (SEQ | 9, 10, 13, 16, 17, 20, | 13, 27, | 27, 55 | 34-36 |
| | ID | 23, 24, 27, 30, 31, 40, | 44, 57, | | |
| | NO: 17) | 41, 43, 44, 45, 47, 48, | 58 | | |
| | | 50, 51, 52, 54, 55, 57, | | | |
| | | 58, 59, 61, 62 | | | |
| | C (SEQ | 9, 10, 13, 16, 17, 20, | 10, 13, | 27, 61 | 36-39 |
| | ID | 23, 24, 27, 30, 31, 34, | 27, 50, | | |
| | NO: 18) | 35, 46, 47, 50, 51, 53, | 65 | | |
| | | 54, 56, 57, 58, 60, 61, | | | |
| | | 63, 64, 65, 67, 68 | | | |
| DHT07 | A (SEQ | 8, 9, 10, 12, 13, 15, | 9, 13, | 9, 16, 20, 27 | 37-39 |
| | ID | 16, 17, 19, 20, 22, 23, | 16, 23, | | |
| | NO: 19) | 24, 26, 27, 29, 30, 31, | 54 | | |
| | | 33, 34, 35, 46, 50, 53, | | | |
| | | 54, 57, 60, 64, 67 | | | |
| | B (SEQ | 10, 13, 17, 20, 21, 24, | 20, 47, | n/a | 33-36 |
| | ID | 27, 31, 40, 41, 43, 44, | | | |
| | NO: 20) | 47, 48, 50, 51, 54, 55, | 54, 62 | | |
| | | 57, 58, 61, 62 | | | |
| | C (SEQ | 9, 10, 12, 13, 16, 19, | 13, 27, | n/a | 36-38 |
| | ID | 20, 23, 24, 26, 27, 29, | 66 | | |
| | NO: 21) | 30, 31, 33, 34, 45, 47, | | | |
| | | 48, 52, 55, 56, 59, 62, | | | |
| | | 63, 66 | | | |
| DHT08 | A (SEQ | 9, 12, 16, 19, 23, 26, | 26, 27, | n/a | 35-38 |
| | ID | 27, 30, 33, 42, 43, 45, | 49, 53, | | |
| | NO: 22) | 46, 49, 50, 52, 53, 56, | 60, 61 | | |
| | | 57, 59, 60, 61, 63, 64 | | | |
| | B (SEQ | 9, 10, 13, 16, 20, 23, | 20, 51, | 46 | 37-39 |
| | ID | 27, 30, 34, 43, 44, 46, | | | |
| | NO: 23) | 47, 50, 51, 53, 54, 56, | 57, 64 | | |
| | | 57, 58, 60, 61, 63, 64, | | | |
| | | 65 | | | |
| | C (SEQ | 8, 9, 12, 16, 19, 23, | 12, 43, | 12, 19, 43 | 33-35 |
| | ID | 26, 30, 39, 40, 42, 43, | 50, 57 | | |
| | NO: 24) | 44, 46, 47, 49, 50, 53, | | | |
| | | 54, 56, 57, 58, 60, 61 | | | |
| DHT09 | A (SEQ | 9, 10, 12, 13, 16, 17, | 9, 16, | 60 | 33-35 |
| | ID | 19, 20, 21, 23, 24, 26, | 28, 60 | | |
| | NO: 25) | 27, 28, 30, 31, 43, 46, | | | |
| | | 49, 50, 53, 56, 57, 60 | | | |
| | B (SEQ | 8, 9, 10, 12, 13, 15, | 9, 16, | 23 | 37-39 |
| | ID | 16, 17, 19, 20, 22, 23, | 19, 23, | | |
| | NO: 26) | 24, 26, 27, 29, 30, 31, | 61 | | |
| | | 33, 34, 46, 50, 53, 57, | | | |
| | | 60, 61, 64, 67 | | | |
| | C (SEQ | 9, 10, 12, 13, 14, 16, | 10, 17, | 9, 16, 30 | 37-39 |
| | ID | 17, 18, 19, 20, 21, 23, | 18, 23, | | |
| | NO: 27) | 24, 26, 27, 28, 30, 31, | 50 | | |
| | | 33, 46, 47, 50, 53, 54, | | | |
| | | 57, 60, 61, 64, 67, 68 | | | |
| DHT10 | A (SEQ | 9, 13, 16, 20, 23, 27, | 27, 50, | n/a | 37-39 |
| | ID | 30, 34, 42, 43, 46, 47, | 57, 64 | | |
| | NO: 28) | 49, 50, 51, 53, 54, 56, | | | |
| | | 57, 60, 61, 63, 64 | | | |
| | B (SEQ | 10, 14, 17, 21, 24, 28, | 17, 44, | 40, 44, 58 | 34-36 |
| | ID | 31, 40, 41, 44, 45, 47, | 54, 58 | | |
| | NO: 29) | 48, 50, 51, 52, 54, 55, | | | |
| | | 57, 58, 59, 61, 62 | | | |
| | C (SEQ | 10, 13, 17, 20, 24, 27, | 20, 24, | n/a | 35-38 |
| | ID | 31, 34, 40, 41, 43, 44, | 48, 51, | | |
| | NO: 30) | 45, 47, 48, 50, 51, 52, | 62 | | |
| | | 54, 55, 57, 58, 59, 61, | | | |
| | | 62 | | | |

In one embodiment, mutations in polypeptide residues relative to the reference sequence are conservative amino acid substitutions. As used herein, a “conservative amino acid substitution” means a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Particular conservative substitutions include, but are not limited to, Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In another embodiment, the disclosure provides fusion proteins comprising
(a) the polypeptide of any embodiment or combination of embodiments herein;
(b) a second polypeptide; and
(c) an optional amino acid linker linking the polypeptide and second polypeptide.

As described herein the proteins may be fused to any further polypeptide domain as suitable for an intended purpose. As described above, the polypeptides have the ability to sustain rigid helical fusions to monomeric repeat proteins (i.e., exemplary second polypeptides)), enabling the incorporation of arms that can be extended to provide three new elongated connection points. In other embodiments, the second protein may be, for example, an antigen or other protein to be displayed on heteropolymers formed from the fusion proteins. In another embodiment, the second polypeptide may comprise an antibody or antigen-binding fragment thereof. In one embodiment, the second polypeptide comprises a helical repeat protein or a protein with mixed alpha helix/beta sheet secondary structure. In various non-limiting embodiments, the fusion protein comprises an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of sequences in Tables 5 and 6 (SEQ ID NO:48-50, 52, 54, 58, 60, 61, 63, 64, 66, 67, 70, 72, 73, 74, 80, 83, 86, 87, 92, 94, 97, 99-102, 105, 106, 108, 110, 111, 113, 114, 116, 117, 119, and 120). These embodiments comprise fusions to monomeric repeat proteins, as described in the examples that follow.

TABLE 5
Fusion arms

| | | Fusion | | Seq ID |
|---|---|---|---|---|
| Name | State | chain | Design sequences | combos |
| DHT01- | ABC | A | DEEEKLRESLEEQKKRVKEIEREHEEIREALKRLQQSVLRQAVRLAK | 48 + 49 + 50 |
| 4arm- | | | LGDDSEMINRIVKDLAEQAKEATDKREVIKIVKALAELAKNSTDSEL | |
| 01 | | | VNEIVKQLAEVAKEATDKELVIYIVKILAELAKQSTDSELVNEIVKQ | |
| | | | LEEVAKEATDKELVEHIEKILEELKKQSTD (SEQ ID NO: 48) | |
| | | B | SEQERREKAKEEVRELNEEFKEAEKRFRKLQEETKKALEEVEELNQR | |
| | | | FQEALTEVAAKKLALEISRVIKTLKESGSSYEEIAEIVARIVAEIVE | |
| | | | ALKRSGASEKDIATIVAAIISAVIQTLKESGSSYEVIAEIVARIVAE | |
| | | | IVEALKRSGTSEDEIAEIVARVISEVIRTLKESGSSYEVIKEIVQRI | |
| | | | VEEIVEALKRSGTSEDEINEIVRRVKSEVERTLKESGSS (SEQ ID | |
| | | | NO: 49) | |
| | | C | MSSDEEEARELIERAKEAAERAQEAAERTGDPRVRELARELKRLAQE | |
| | | | AAEEVKRDPSSSDVNEALKLIVEAIEAAVRALEAAERTGDPEVRELA | |
| | | | RELVRLAVEAAEEVQRNPSSSDVNEALKLIVEAIEAAVRALEAAERT | |
| | | | GDPEVRELARRMVRAAVRAAEVVQRNPSDEEANEMLKQIVKLAQQAV | |
| | | | EMLREAEESGDPEKREKAKQTVEQAIREAELLLLIWEWKRLRERLQE | |
| | | | LEEQHRELQEELKELREKWKRLKERLERLRLELRLEEMARRARNATD | |
| | | | SEQINEIVKQLAEIAKEATDKRAVIKIVKILAELAKKSTDSELVNEI | |
| | | | VKQLAEVAKEATDKELVIYIVKILAELAKQSTDSELVNEIVKQLEEV | |
| | | | AKEATDKELVEHIEKILEELKKQSTD (SEQ ID NO: 50) | |
| DHT01- | ABC | A | DEEEKLRESLEEQKKRVKEIEREHEEIREALKRLQQSVLRQAVRLAK | 48 + 52 + 50 |
| 4arm- | | | LGDDSEMINRIVKDLAEQAKEATDKREVIKIVKALAELAKNSTDSEL | |
| 02 | | | VNEIVKQLAEVAKEATDKELVIYIVKILAELAKQSTDSELVNEIVKQ | |
| | | | LEEVAKEATDKELVEHIEKILEELKKQSTD (SEQ ID | |
| | | | NO: 48) | |
| | | B | MSSDEEEARELIERAKEAAERAQEAAERTGDPRVRELARELKRLAQE | |
| | | | AAEEVKRDPSSSDVNEALKLIVEAIEAAVRALEAAERTGDPEVRELA | |
| | | | RELVRLAVEAAEEVQRNPSSSDVNRALKLIVRAIEAAVQLLEHAEKS | |
| | | | GDPEVRELARELVRRLVEAAEEVQRNPSDEQKNTQLRRLIAKAEVWL | |
| | | | LNEEFKEAEKRFRKLQEETKKALEEVEELNQRFEEALEEVERIKRRQ | |
| | | | K (SEQ ID NO: 52) | |
| | | C | MSSDEEEARELIERAKEAAERAQEAAERTGDPRVRELARELKRLAQE | |
| | | | AAEEVKRDPSSSDVNEALKLIVEAIEAAVRALEAAERTGDPEVRELA | |
| | | | RELVRLAVEAAEEVQRNPSSSDVNEALKLIVEAIEAAVRALEAAERT | |
| | | | GDPEVRELARRMVRAAVRAAEVVQRNPSDEEANEMLKQIVKLAQQAV | |
| | | | EMLREAEESGDPEKREKAKQTVEQAIREAELLLLIWEWKRLRERLQE | |
| | | | LEEQHRELQEELKELREKWKRLKERLERLRLELRLEEMARRARNATD | |
| | | | SEQINEIVKQLAEIAKEATDKRAVIKIVKILAELAKKSTDSELVNEI | |
| | | | VKQLAEVAKEATDKELVIYIVKILAELAKQSTDSELVNEIVKQLEEV | |
| | | | AKEATDKELVEHIEKILEELKKQSTD (SEQ ID NO: 50) | |
| DHT03_ | AB | A | SEKEKVEELAQRIREQLPDTELAREAQELADEARKSDDSEALKVVYL | 54 + 55 + 9, or |
| 1arm_ | C | | ALRIVQQLPDTELAREALELAKEAVKSTDSEALKVVYLALRIVQQLP | |
| A21/B/C | | | DTELARLALELAKKAVEMTAQEVLEIARAALKAAQAFPNTELAELML | |
| | | | RLAEVAARVMKELERNDEEIKKSDELDDESLLEDIVELLKEIIKLWK | |
| | | | ILVEVSDVMLKLIS (SEQ ID NO: 54) | |
| | | | or | |
| | | | SEKEKVEELAQRIREQLPDTELAREAQELADEARKSDDSEALKVVYL | 63 + 55 + 9, or |
| | | | ALRIVQQLPDTELAREALELAKEAVKSTDSEALKVVYLALRIVQQLP | |
| | | | DTELARLALELAKKAVEMTAQEVLEIAKAALKAAQAFPNTELAELML | |
| | | | RLAEVAARVMKELERNDEEIKKSDELDDESLLEDIVELLKEIIKLWK | |
| | | | ILVEVSDVMLKLIS (SEQ ID NO: 63) | |
| | | | or | |
| | | | SEKEKVEELAQRIREQLPDTELAREAQELADEARKSDDSEALKVVYL | 66 + 55 + 9, or |
| | | | ALRIVQQLPDTELAREALELAKEAVKSTDSEALKVVYLALRIVQQLP | |
| | | | DTELARLALELAKKAVEMTAQEVLAIAKAALKAAQAFPNTELAELML | |
| | | | RLAEVAARVMKELERNDEEIKKSDELDDESLLEDIVELLKEIIKLWK | |
| | | | ILVEVSDVMLKLIS (SEQ ID NO: 66) | |
| | | | or | |
| | | | SEKEKVEELAQRIREQLPDTELAREAQELADEARKSDDSEALKVVYL | 72 + 55 + 9 |
| | | | ALRIVQQLPDTELAREALELAKEAVKSTDSEALKVVYLALRIVQQLP | |
| | | | DTELARLALELAKKAVEMTAQEVLAIAKAALKAAQAFPNTELAELML | |
| | | | RLAEVAARVMKELERNDEEIKKSDELDDASLLADIVRLLAEIIKLWK | |
| | | | ILVEVSDVMLKLIS (SEQ ID NO: 72) | |
| DHT03_ | ABC | B | SEKEKVEELAQRIREQLPDTELAREAQELADEARKSDDSEALKVVYL | 7 + 58 + 59, or |
| 1arm_ | | | ALRIVQQLPDTELAREALELAKEAVKSTDSEQLEVVRLALEIVQLAP | |
| A/B21/C | | | DTRLARAALKLAKEAVKSTDQEELKKVKAILRVASEVLKLEEEAKKS | |
| | | | QEEVERLKQEVEKASKAGLDHEGDSRIFKKIHDVVTKQIKVIIRLIE | |
| | | | VYVRLVEIIL (SEQ ID NO: 58) | |
| | | | or | |
| | | | SEKEKVEELAQRIREQLPDTELAREAQELADEARKSDDSEALKVVYL | 7 + 73 + 59 |
| | | | ALRIVQQLPDTELAREALELAKEAVKSTDSEQLEVVRLALEIVQLAP | |
| | | | DTRLARAALKLAKEAVKSTDQEELKKVKAILRVASEVLKLEEEAKKS | |
| | | | QEEVERLKQEVEKASKAGLDHEGDSRIFKKIHDVVTKQIKVILRLIA | |
| | | | VYAELVAIIG (SEQ ID NO: 73) | |
| DHT03_ | ABC | A | SEKEKVEELAQRIREQLPDTELAREAQELADEARKSDDSEALKVVYL | 60 + 61 + 59 |
| 2arm_ | | | ALRIVQQLPDTELAREALELAKEAVKSTDSEALKVVELALKIVQQLP | |
| A21/B21/ | | | DTELAKEALKLAKEAVKSTDSEALKVVELALEIVQQLPDTELAKEAL | |
| C | | | ELAEEAVKSTDSEALKVVKLALEIVQQLPDTELAREALELAKEAVKS | |
| long | | | TDSEALKVVYLALRIVQQLPDTELARLALELAKKAVEMTAQEVLEIA | |
| | | | RAALKAAQAFPNTELAELMLRLAEVAARVMKELERNDEEIKKSDELD | |
| | | | DESLLEDIVELLKEIIKLWKILVEVSDVMLKLIS (SEQ ID | |
| | | | NO: 60) | |
| | | B | SEKEKVEELAQRIREQLPDTELAREAQELADEARKSDDSEALKVVYL | |
| | | | ALRIVQQLPDTELAREALELAKEAVKSTDSEALKVVELALKIVQQLP | |
| | | | DTELAKEALELAKEAVKSTDSEALKVVELALEIVQQLPDTELAKEAL | |
| | | | KLAKEAVKSTDSEALKVVYLALRIVQQLPDTELAREALELAKEAVKS | |
| | | | TDSEQLEVVRLALEIVQLAPDTRLARAALKLAKEAVKSTDQEELKKV | |
| | | | KAILRVASEVLKLEEEAKKSQEEVERLKQEVEKASKAGLDHEGDSRI | |
| | | | FKKIHDVVTKQIKVILRLIAVYAELVAIIG (SEQ ID NO: 61) | |
| DHT03_ | ABC | B | TEEKIAKEISRIAEESKKRIEELARKADNKTTETEVDKAIEKIAKLA | 63 + 64 + 65 |
| 2arm_ | | | REAIKRIEDLAKNLASEEFMARAISAIAELAKKAIEAIYRLADNHTT | |
| A21/Bt18/ | | | DTFMAKAIEAIAELAKEAIKAIADLAKNHTTEEFMARAISAIAELAK | |
| C | | | KAIEAIYRLADNHTTDTFMAKAIEAIAELAKEAIKAIADLAKNHTTE | |
| | | | EFMAKAISAIMELAVKAILAIARLASNHTSQTYREKAREAVEKIART | |
| | | | AEKAIEDLAKNITTEEYKERAKKARAIVRVMAEVAKLLIEAVESQEE | |
| | | | VERLKQEVEKASKAGLDHEGDSRIFKKIHDVVTKQIKVIIRLIAVYA | |
| | | | ELVAIIG (SEQ ID NO: 64) | |
| DHT03_ | ABC | B | SEEVNERVKQLAEKAKEATDKEEVIEIVKELAELAKQSTDSELVNEI | 66 + 67 + 56 |
| 2arm_ | | | VKQLAEVAKEATDKELVIYIVKILAELAKQSTDSELVNEIVKQLAEV | |
| A21/B14/ | | | AKEATDKELVKYIAEILDELAKQSTDDELRKEIIKQMLEVAAEAKDK | |
| C | | | ELIEDLVKIMVDMLEELAKSSQEEVERLKQEVEKASKAGLDHEGDSR | |
| | | | IFKKIHDVVTKQIKIIILLIAVYAMLVAAIG (SEQ ID NO: 67) | |
| DHT03_ | ABC | B | NDEKRKRAEKALQRAQEAEKKGDVEEAVRAAQEAVRAAKESGDNDVL | 63 + 70 + 56 |
| 2arm_ | | | RKVAEQALRIAKEAEKQGNVEVAVKAARVAVEAAKQAGDNDVLRKVA | |
| A21/B62/ | | | EQALRIAKEAEKQGNAEVAAKAAKVAAEAAAQAGDDDVLKKVIEVAN | |
| C | | | RIAKVANKLGDIKTAVEALEIAAEASQVMVELLKQEVEKASKAGLDH | |
| | | | EGDSRIFKKIHEVVTIQIKIIIILIAVYALLVAIIG (SEQ ID | |
| | | | NO: 70) | |
| DHT03_ | ABC | C | KQKEAIKVYLELLEVHSRVLKALIEQIKLFIELIMEPDEDLADKVRK | 72 + 73 + 74 |
| 3arm_ | | | SSEELKKIIKEVEKILRKVELKLRIDKARKLVRERPGSNLAKKALEE | |
| A21/B21/ | | | MLRAAEEAAKLPDPEALKQAVKAAEEVVREQPGSNLAKKALEIILRA | |
| C53 | | | AEELAKLPDPEALKEAVKAAEKVVREQPGSELAKKALEIIERAAEEL | |
| | | | KKSPDPEAQKEAKKAEQKVREERPG (SEQ ID NO: 74) | |
| DHT03_ | ABC | C | KQKEAIKVYLELLEVHSRVLKALIEQIKLFIELIMEPDEDLADKVRK | 72 + 73 + 80 |
| 3arm_ | | | SSEELKKIIKEVEKILKKVRKILIRLKARRVEKEAKELAKELDSEEA | |
| A21/B21/ | | | KKVVERIKEAAEAAERAAEQGKDEVAELALKVLREAIELAKENRSEE | |
| C59 | | | ALKVVLEIARAALAAAQAAEEGKTEVAKLALKVLEEAIELAKENRSE | |
| | | | EALKVVLEIARAALAAAQAAEEGKSDEARDALRRLEEAIEEAKENRS | |
| | | | KESLEKVREEAKEAEQQAEDAREGK (SEQ ID NO: 80) | |
| DHT03_ | ABC | C | KQKEAIKVYLELLEVHSRVLKALIEQIKLFIELIMEPDEDLADKVRK | 72 + 73 + 83 |
| 3arm_ | | | SSEELKKIIKEVEKILKKVSQIVAELALKRAREAEKKGDVEEAVRAA | |
| A21/B21/ | | | QKAVKAAKDAGDNDMLRKVAEVALRIAKEAEKQGNVEVAVKAARVAV | |
| C62 | | | EAAKQAGDNDVLRKVAEQALRIAKEAEKQGNVEVAVKAARVAVEAAK | |
| | | | QAGDQDVLRKVSEQAERISKEAKKQGNSEVSEEARKVADEAKKQTGD | |
| | | | (SEQ ID NO: 83) | |
| DHT03_ | ABC | C | KQKEAIKVYLELLEVHSRVLKALIEQIKLFIELIMEPDEDLADKVRK | 72 + 73 + 86 |
| 3arm_ | | | SSEELKKIIEEVKQILRLVEAELMRQKAEEAIKKARKTGDPELLRKA | |
| A21/B21/ | | | LELLEEAVRIVEEAIKKDPDDDEAVELAVRLARMLKKVAEELQERAK | |
| C82 | | | KTGDPELLKLALRALEVAVRAVELAIKSNPDNDEAVETAVRLARELK | |
| | | | KVAEELQERAKKTGDPELLKLALRALEVAVRAVELAIKSNPDNEEAV | |
| | | | ETAKRLAEELRKVAELLEERAKETGDPELQELAKRAKEVADRARELA | |
| | | | KKSNPNN (SEQ ID NO: 86) | |
| DH 05_ | ABC | A | NDEKRKRAEKALQRAQEAEKKGDVEEAVRAAQEAVRAAKESGDNDVL | 87 + 88 + 89, |
| 1arm_ | | | RKVAEQALRIAKEAEKQGNVEVAVKAARVAVEAAKQAGDNDVLRKVA | or |
| A62/B/C | | | EQALRIAKEAEKQGNVKVAADAVKVAVEAATQAGDQDVLRKASEQAK | |
| | | | KVAKEAKKKGDHKVAVKATTLAVKAEFAAIQEEIKKLEEEAKKAEKG | |
| | | | NGKEEIKELLLRLSELLARSLQLLAQQIEAIAKLIRG (SEQ ID | |
| | | | NO: 87) | |
| | | | or | |
| | | | NDEKRKRAEKALQRAQEAEKKGDVEEAVRAAQEAVRAAKESGDNDVL | 99 + 88 + 89 |
| | | | RKVAEQALRIAEEAEKQGNVEVAVKAARVAVEAAKQAGDNDVLRKVA | |
| | | | EQALRIAEEAEKQGNVKVAADAVEVAVEAATQAGDQDVLRKASEQAK | |
| | | | KVAKEAKKKGDHKVAVKATTLAVKAEFAAIQEEIKKLEEEAKKAEKG | |
| | | | NGKEEIKELLLELSELLARSLQLLAQQIEAIAKLIRG (SEQ ID | |
| | | | NO: 99) | |
| DHT05_ | ABC | C | NDEKRKRAEKALQRAQEAEKKGDVEEAVRAAQEAVRAAKESGDNDVL | 13 + 88 + 92, |
| 1arm_ | | | RKVAEQALRIAKEAEKQGNVEVAVKAARVAVEAAKQAGDNDVLRKVA | or |
| A/B/C62 | | | EQALRIAEEALKQGNTEVAKKANRVAREAAKQAGDQDVLRKVEKMEL | |
| | | | KIRLAELLRKIRELIKEIEEVIKEYQKLKERGDGGEKEEKELIQANL | |
| | | | RLLKLHTRLLKLYLELLKLIIRMG (SEQ ID NO: 92) | |
| | | | or | |
| | | | NDEKRKRAEKALQRAQEAEKKGDVEEAVRAAEEAVRAAKESGDNDVL | 13 + 88 + 101 |
| | | | RKVAEQALRIAKEAEKQGNVEVAVKAARVAVEAAKQAGDNDVLRKVA | |
| | | | EQALRIAEEALKQGNTEVAKEANRVAREAAKQAGDQDVLRKVEKMEE | |
| | | | KIELAELLEKIRELIKEIEEVIKEYQKLKERGDGGEKEEKELIQANL | |
| | | | RLLKLHTELLKLYLELLKLIIRQG (SEQ ID NO: 101) | |
| DHT05_ | ABC | B | SEVEKRLLEIHREVAESHRILVEVHEALIEALRGNSEEAKEKLKELV | 87 + 94 + 95 |
| 2arm_ | | | KKLEKIIKEEEELLKKLEKIVVQALAKAAKQATDSRQVNEIVKQLAE | |
| A62/B14/ | | | IAKEATDKRLVIEIVKILAELAKQSTDSELVNEIVKQLAEVAKEATD | |
| C | | | KELVIYIVKILAELAKQSTDSELVNEIVKQLEEVAKEATDKELVEHI | |
| | | | EKILEELKKQSTD (SEQ ID NO: 94) | |
| DHT05_ | ABC | B | SETEKRLLEIHRRVAESHRILVEVHEALIEALRGNSEEAKEKLKELV | 87 + 97 + 98 |
| 2arm_ | | | KKLEKIIREEEKLTVELAAKVAKSADDSEKVNEIVKTLAEIAKEATD | |
| A62/B14 | | | KEQVIRIVKILAELAKQSTDSELVNEIVKQLAEVAKEATDKELVIYI | |
| (2)- | | | VDILLKLAEQADDDELVEEIRKQLEEVAKEATDKELVEIIKAVIVLL | |
| LHD101 | | | VIISVVARMGVTMEIHKSGREVKVVIKGLHESQQEQLLEAVLRAAEE | |
| B14/C | | | AGVRVRIRFKGDTVTIVVRG (SEQ ID NO: 97) | |
| DHT05_ | AB | B | SEVEKRLLEIHRQVAESHRILVEVHEALIEALRGNSEEAKEKLKELV | 99 + 100 + 101 |
| 3arm_ | C | | KKLEKIIRQEELLLQLLEALVKALQAKKEGDPELVLEAAKDALRVAE | |
| A62/B71/ | | | QAAKEGDKEMFKQAAELALWLAKQLVEVASKEGDPELVLEAAKVALR | |
| C62 | | | VAELAAKNGDKEVFKKAAESALEVAKRLVEVASKEGDPELVEEAAKV | |
| | | | AEEVRKLAKKQGDEEVYEKARETAREVKEELKRVREEK (SEQ ID | |
| | | | NO: 100) | |

TABLE 6
Cyclic oligomers

| | | Fusion | | Seq ID |
|---|---|---|---|---|
| Name | State | chain | Design Sequences | combos |
| C2- | A2 | A | DEEEKLRESLEEQKKRVKEIEREHEEIREALKRLQQSVLRQAVRLAKLGDDSEMIRR | 102 + 50 |
| DHT01- | B2 | | IVKDLMQQAIEATDEREVEKIKKAAEELSKNSTDEDLKKQIKAVQELVKAVETVREA | |
| 01 | | | KKQGDPKKVLKAAEIAMEVATWAAERNDEMIFKLAAKMAKEVAERLKEVAKKENDSA | |
| | | | LELAAEAIKAAVEALEAALRTGDPRVRELAKELVRLAKEAAEEATRDSKDSDKNEAL | |
| | | | KLIVEAIKAAVRALEAAERTGDPEVRELARELVRLAVEAAEEVLRNPSSSDVNRALK | |
| | | | LIVRAIEAAVQLLEHAEKSGDPEVRELARELVRRLVEAAEEVQRNPSDEQKNTQLRR | |
| | | | LIAKAEVWLLNEEFKEAEKRFRKLQEETKKALEEVEELNQRFEEALEEVERIKRRQK | |
| | | | (SEQ ID NO: 102) | |
| | | B | MSSDEEEARELIERAKEAAERAQEAAERTGDPRVRELARELKRLAQEAAEEVKRDPS | |
| | | | SSDVNEALKLIVEAIEAAVRALEAAERTGDPEVRELARELVRLAVEAAEEVQRNPSS | |
| | | | SDVNEALKLIVEAIEAAVRALEAAERTGDPEVRELARRMVRAAVRAAEVVQRNPSDE | |
| | | | EANEMLKQIVKLAQQAVEMLREAEESGDPEKREKAKQTVEQAIREAELLLLIWEWKR | |
| | | | LRERLQELEEQHRELQEELKELREKWKRLKERLERLRLELRLEEMARRARNATDSEQ | |
| | | | INEIVKQLAEIAKEATDKRAVIKIVKILAELAKKSTDSELVNEIVKQLAEVAKEATD | |
| | | | KELVIYIVKILAELAKQSTDSELVNEIVKQLEEVAKEATDKELVEHIEKILEELKKQ | |
| | | | STD (SEQ ID NO: 50) | |
| C2- | A2 | A | MSSDEEEARELIERAKEAAERAQEAAERTGDPRVRELARELKRLAQEAAEEVKRDPS | 50 + 105 |
| DHT01- | B2 | | SSDVNEALKLIVEAIEAAVRALEAAERTGDPEVRELARELVRLAVEAAEEVQRNPSS | |
| 02 | | | SDVNEALKLIVEAIEAAVRALEAAERTGDPEVRELARRMVRAAVRAAEVVQRNPSDE | |
| | | | EANEMLKQIVKLAQQAVEMLREAEESGDPEKREKAKQTVEQAIREAELLLLIWEWKR | |
| | | | LRERLQELEEQHRELQEELKELREKWKRLKERLERLRLELRLEEMARRARNATDSEQ | |
| | | | INEIVKQLAEIAKEATDKRAVIKIVKILAELAKKSTDSELVNEIVKQLAEVAKEATD | |
| | | | KELVIYIVKILAELAKQSTDSELVNEIVKQLEEVAKEATDKELVEHIEKILEELKKQ | |
| | | | STD (SEQ ID NO: 50) | |
| | | B | DEEEKLRESLEEQKKRVKEIEREHEEIREALKRLQQSVLRQAVRLAKLGDDSEMIQR | |
| | | | IVEDLARQAMEATDDREVEKIEKALDELAKNSTDEELKKVIKVAKTLVRVAKLVKEA | |
| | | | EELLRQAKEKGSEEDLEKALRTAEEAAREAKKVLEEAEKAQIAEAALLAVELVVRVA | |
| | | | ELLLRIAKESGSEEALERALRVAEEAARLAKRVLELAEKQGDPEVALRAVELVVRVA | |
| | | | ELLLRIAKESGSSEALTRAWRVAIEAFQLAVRVLKLAQKQNDSEVARRATELMKRVA | |
| | | | ELMERIARESGSSAFKAAAEALEAAARTNDKEVVDLARELARLAIEALEEVERNSKS | |
| | | | SDVNRALKLILRAIKAAVQLLEHAEKSGDPEVRELARELVRRLVEAAEEVQRNPSDE | |
| | | | QKNTQLRRLIAKAEVWLLNEEFKEAEKRFRKLQEETKKALEEVEELNQRFEEALEEV | |
| | | | ERIKRRQK (SEQ ID NO: 105) | |
| C2- | A2 | A | MGDEEEKLRESLEEQKKRVKEIEREHEEIREALKRLQQSVLRQAVRLAKLGDDSRMI | 106 + 50 |
| DHT01- | B2 | | ASIIRDLVKQALRATDEREQKKIKKALEELAQNATDEALKLLIEAAKSLLRAAEASE | |
| 03 | | | RGDEEEFRKAAEKALELAKRLVEIAKKQSLPEFVLAAAEIALAVAELAAKNNDSEVL | |
| | | | EKALRSAAEVAQRLLEVAEKENNQELVEEAAKILKRALEIVEKAAKKLKERNDQSSD | |
| | | | VNRALTLIRRAQEAADQLLEHARKSGDPEVWELAIRLVARLVEAAAKVAANMSDEQQ | |
| | | | NTQLRRLIAKAEVWLLNEEFKEAEKRFRKLQEETKKALEEVEELNQRFEEALEEVER | |
| | | | IKRRQKGGSGWSHPQFEK (SEQ ID NO: 106) | |
| | | B | MSSDEEEARELIERAKEAAERAQEAAERTGDPRVRELARELKRLAQEAAEEVKRDPS | |
| | | | SSDVNEALKLIVEAIEAAVRALEAAERTGDPEVRELARELVRLAVEAAEEVQRNPSS | |
| | | | SDVNEALKLIVEAIEAAVRALEAAERTGDPEVRELARRMVRAAVRAAEVVQRNPSDE | |
| | | | EANEMLKQIVKLAQQAVEMLREAEESGDPEKREKAKQTVEQAIREAELLLLIWEWKR | |
| | | | LRERLQELEEQHRELQEELKELREKWKRLKERLERLRLELRLEEMARRARNATDSEQ | |
| | | | INEIVKQLAEIAKEATDKRAVIKIVKILAELAKKSTDSELVNEIVKQLAEVAKEATD | |
| | | | KELVIYIVKILAELAKQSTDSELVNEIVKQLEEVAKEATDKELVEHIEKILEELKKQ | |
| | | | STD (SEQ ID NO: 50 | |
| C2- | A2 | A | DEEEKLRESLEEQKKRVKEIEREHEEIREALKRLQQSVLRQAVRLAKLGDDSEMINR | 108 + 52 |
| DHT01- | B2 | | IVKDLAEQAKEATDKREVIKIVKALAELAKNSTDRELINEIAKQLAEVAKEATDEEL | |
| 04 | | | QRYIAKILIELAEQAIKEAERSLREGNPEKAREDVRLALELVRLLLEIAQALNLLEV | |
| | | | LEEAARLAFEVARVAKEVGSPETARQARETAERLLEWAMKMIEEKAKKEKDRNDQSS | |
| | | | DVNEAAKLIREAAEAAQRAREAASRTGDPEVKDLAWRMVEAALKAAAVVATNPSDEE | |
| | | | ANEMLKQIVKLAQQAVEMLREAEESGDPEKREKAKQTVEQAIREAELLLLIWEWKRL | |
| | | | RERLQELEEQHRELQEELKELREKWKRLKERLERLRLELRLEEMARRARNATDSEQI | |
| | | | NEIVKQLAEIAKEATDKRAVIKIVKILAELAKKSTDSELVNEIVKQLAEVAKEATDK | |
| | | | ELVIYIVKILAELAKQSTDSELVNEIVKQLEEVAKEATDKELVEHIEKILEELKKQS | |
| | | | TD (SEQ ID NO: 108) | |
| | | B | MSSDEEEARELIERAKEAAERAQEAAERTGDPRVRELARELKRLAQEAAEEVKRDPS | |
| | | | SSDVNEALKLIVEAIEAAVRALEAAERTGDPEVRELARELVRLAVEAAEEVQRNPSS | |
| | | | SDVNRALKLIVRAIEAAVQLLEHAEKSGDPEVRELARELVRRLVEAAEEVQRNPSDE | |
| | | | QKNTQLRRLIAKAEVWLLNEEFKEAEKRFRKLQEETKKALEEVEELNQRFEEALEEV | |
| | | | ERIKRRQK (SEQ ID NO: 52) | |
| C3- | A3 | A | TTNFHLINGSEEARQLIQKAVEAISKKEGTEVHFEKSDGTLEIRVKNLHPRQEDLIK | 110 + 111 + |
| DHT03- | B3 | | KFIEALLLALVAKGELEQAEKEGDAEVALRAVEKVVRVAELLLRLAKEAGSEEALKA | 59 |
| 01 | C3 | | ALEIAEQAARLAKRVLELAEKQGDAEVALKAVLLVVIVAALLVKIAKESGSEEAKER | |
| | | | ATRVAREASRLADRVEELARKQNNEDVTLAAKVLKLAADIIAQLMNTELAEEAAQLA | |
| | | | TEALKATDREQLEVVRLALEIVQLAPDTRLARAALKLAKEAVKSTDQEELKKVKAIL | |
| | | | RVASEVLKLEEEAKKSQEEVERLKQEVEKASKAGLDHEGDSRIFKKIHDVVTKQIKV | |
| | | | ILRLIAVYAELVAIIG (SEQ ID NO: 110) | |
| | | B | NTHFIVVHGGEEARQLAETAVREISKKEGTEVRFEKKDGLLSIEVKNLSEELQRLIQ | |
| | | | ELLQLLVRLAALLEAVRAVEEAIKRNPDNDEAVETAVRLARELKKVAELLQELAKKA | |
| | | | GVPAILRGALLALEVAVRAVELAIKSNPDNDEAVETAVRLAVVKLALEIVQQLPDTE | |
| | | | LAREALELAKEAVKSTDSEALKVVYLALRIVQQLPDTELARLALELAKKAVEMTAQE | |
| | | | VLEIARAALKAAQAFPNTELAELMLRLAEVAARVMKELERNDEEIKKSDELDDESLL | |
| | | | EDIVELLKEIIKLWKILVEVSDVMLKLIS (SEQ ID NO: 111) | |
| C3- | A3 | A | SSIFLLSNVDESARQLAEELVREISKKEGTEVRFEKDDGFLTIEVKNLSEERLREIA | 113 + 114 + |
| DHT03- | B3 | | RALQLIVDVANAERVVRERPGSNLAKKALEIILRAAEELAKLTLEASLKAAQIAAEL | 59 |
| 02 | C3 | | VVREQPGSNLAEKAKTIILQARAAELAIRIEEQLKDTELAKEAKELATEAIKAQDKE | |
| | | | ALKVVELALKIVQQLPDTELAKEALKLAKEAVKSTDSEALKVVELALEIVQQLPDTE | |
| | | | LAKEALELAEEAVKSTDSEALKVVKLALEIVQQLPDTELAREALELAKEAVKSTDSE | |
| | | | ALKVVYLALRIVQQLPDTELARLALELAKKAVEMTAQEVLEIARAALKAAQAFPNTE | |
| | | | LAELMLRLAEVAARVMKELERNDEEIKKSDELDDESLLEDIVELLKEIIKLWKILVE | |
| | | | VSDVMLKLIS (SEQ ID NO: 113) | |
| | | B | TWQWVLINISEEARQLIEKAVRAISKKEGTEVHFEKDDGVLHIRVKNLHEKRAREIH | |
| | | | KVAKLILEVAAAERIVRERPGSNLAKKALEIILRAAEELAKADVDAALEAAVRAAEK | |
| | | | VVREQPGSNLAKKALEIILRAAEELAKLPDPEALKEAVKAAEKVVREQPNSELAKKA | |
| | | | REIIKRAAEELAKSPDKEAIQEAATALAILIELILSNTELAREAKELATEARKATDS | |
| | | | EAAKVVFLALFIVVQLPDTELAKEALELAKEAVKSTDSEALKVVELALEIVQQLPDT | |
| | | | ELAKEALKLAKEAVKSTDSEALKVVYLALRIVQQLPDTELAREALELAKEAVKSTDS | |
| | | | EQLEVVRLALEIVQLAPDTRLARAALKLAKEAVKSTDQEELKKVKAILRVASEVLKL | |
| | | | EEEAKKSQEEVERLKQEVEKASKAGLDHEGDSRIFKKIHDVVTKQIKVILRLIAVYA | |
| | | | ELVAIIG (SEQ ID NO: 114) | |
| C4- | A4 | A | MGNTHFIVVHGGEEARQLAETAVREISKKEGTEVRFEKKDGLLSIEVKNLSEELQRL | 116 + 117 + |
| DHT03- | B4 | | IQELLQLLVRLAALLEAVRAVEEAIKRNPDNDEAVETAVRLARELKKVAELLQELAK | 59 |
| 01 | C4 | | KAGVPAILRGALLALLVAARAVALAALANSKNDEAREALKRLAEELKKVAKELKERA | |
| | | | KKTKDSELERLALAAELMALAAEIVAQLANTELAREALELATEAIKASDDEALRVVK | |
| | | | LALRIVQQLPDTELARLALELAKKAVEMTAQEVLEIARAALKAAQAFPNTELAELML | |
| | | | RLAEVAARVMKELERNDEEIKKSDELDDESLLEDIVELLKEIIKLWKILVEVSDVML | |
| | | | KLIS (SEQ ID NO: 116) | |
| | | B | MGTTNFHLINGSEEARQLIQKAVEAISKKEGTEVHFEKSDGTLEIRVKNLHPRQEDL | |
| | | | IKKFIEALLLALVAKGELEQAEKEGDAEVALRAVEKVVRVAELLLRLAKEAGSEEAL | |
| | | | KAALEIAEQAARLAARVFTLAVKQNDSDVAKRAVELIKRVAELLKRIAKESGSEEAK | |
| | | | ERAEKVEKAAEVAEIAARILDQLSNTELAKEALKLASEALKSDIKEALEVVRLALRI | |
| | | | VQQLPDTELAREALELAKEAVKSTDSEQLEVVRLALEIVQLAPDTRLARAALKLAKE | |
| | | | AVKSTDQEELKKVKAILRVASEVLKLEEEAKKSQEEVERLKQEVEKASKAGLDHEGD | |
| | | | SRIFKKIHDVVTKQIKVILRLIAVYAELVAIIG (SEQ ID NO: 117) | |
| C4- | A | A | MGNTHFIVVHGGEEARQLAETAVREISKKEGTEVRFEKKDGLLSIEVKNLSEELQRL | 119 + 120 + |
| DHT | 4 | | IQELLQLLVRLAALLEAVRAVEEAIKRNPDNDEAVETAVRLARELKKVAELLQELAK | 59 |
| 03- | B | | KAGVPAILRGALLALEVAVRAVELAIKSNPDNDEAVETAVRLARELLKVAAELAERA | |
| 02 | 4 | | AKTKDEELIKLAQRAIEVAQRAVELAKKSNKDNEEAEKTRLALEAMELALQIFRQLK | |
| | C | | NTELAREAIELATEAAKATDSEALKVVKLALKIVQQLPDTELAKEALELAKEAVKST | |
| | 4 | | DSEALKVVELALEIVQQLPDTELAKEALKLAKEAVKSTDSEALKVVYLALRIVQQLP | |
| | | | DTELAREALELAKEAVKSTDSEQLEVVRLALEIVQLAPDTRLARAALKLAKEAVKST | |
| | | | DQEELKKVKAILRVASEVLKLEEEAKKSQEEVERLKQEVEKASKAGLDHEGDSRIFK | |
| | | | KIHDVVTKQIKVILRLIAVYAELVAIIG (SEQ ID NO: 119) | |
| | | B | MGTTNFHLINGSEEARQIIEKIVEEVARKAGTEVHFEKSDGTLEIRVKNLHEELERR | |
| | | | IKKAIEAALAAQIAKKLAEQIERQLSNTELAKEAKKLATEAEKARDSEAVQVVLLAL | |
| | | | EIVQQLPDTELAKEALELAEEAVKSTDSEALKVVKLALEIVQQLPDTELAREALELA | |
| | | | KEAVKSTDSEALKVVYLALRIVQQLPDTELARLALELAKKAVEMTAQEVLEIARAAL | |
| | | | KAAQAFPNTELAELMLRLAEVAARVMKELERNDEEIKKSDELDDESLLEDIVELLKE | |
| | | | IIKLWKILVEVSDVMLKLIS (SEQ ID NO: 120) | |
| C3- | A3 | | All sequences shown above | 110 + 111 + |
| DHT03- | B3 | | | 86 |
| 01- | C3 | | | |
| DHR82 | | | | |
| C4- | A4 | | All sequences shown above | 116 + 117 + |
| DHT03- | B4 | | | 86 |
| 01- | C4 | | | |
| DHR82 | | | | |

In another aspect, the disclosure provides nucleic acids encoding the polypeptide or fusion protein of any embodiment or combination of embodiments of the disclosure. The nucleic acid sequence may comprise single stranded or double stranded RNA (such as an mRNA) or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the nucleic acids, expression vectors (i.e., episomal or chromosomally integrated), polypeptides, fusion protein, or compositions disclosed herein, wherein the host cells can be either prokaryotic or eukaryotic. The cells can be transiently or stably engineered to incorporate the nucleic acids or expression vector of the disclosure, using techniques including but not limited to bacterial transformations, calcium phosphate co-precipitation, electroporation, or liposome mediated-, DEAE dextran mediated-, polycationic mediated-, or viral mediated transfection.

In a further aspect, the disclosure provides heterotrimers, heterodimers, or heterotetramers comprising polypeptides that comprise an amino acid sequence at least 70%, 75%, 80%, 85%, 90%, 91%, 92%, 93%, 94%, 95%, 96%, 97%, 98%, 99%, or 100% identical to an amino acid sequences combination listed in Table 1, Table 2, Table 3, Table 5, or Table 6, in, wherein 0-7 residues at the N and/or C-terminus of the polypeptides are optional and may be absent and not considered when determining percent identity.

As described above, the heteropolymers can be used as interaction hub building blocks between chains in larger closed structures generated through geometry-aware rigid fusion to themselves and other designed proteins. A notable feature is that these new assemblies can continue to be built out recursively, unlike previously designed rings, as they have outward facing chains with two or more free termini. The number and orientational accessibility of the chain termini found in the heteropolymers enable the display of multiple distinct functional domains for signaling and other potential applications. As illustrated by the larger ring designs, the modularity and orthogonality of the designed protein interfaces makes it possible to combine the heterotrimers with other heteromeric building blocks to construct more diverse nanostructures.

The far right column of each of Tables 1-3 and 5-6 provides the SEQ ID NO of polypeptide combinations to form the heterotrimers, heterodimers, or heterotetramers of this aspect. For example, the first row of Table 1 is shown below, and the far right column shows that polypeptides that comprise an amino acid sequence at least 70%, 75%, 80%, 85%, 90%, 91%, 92%, 93%, 94%, 95%, 96%, 97%, 98%, 99%, or 100% identical to the amino acid sequences of SEQ ID NOS 1, 2, and 3 form an obligate heterotrimer.

| | | | | |
|---|---|---|---|---|
| DHT01 | ABC | A | DEEEKLRESLEEQKKRVKEIEREHEEIREALKRLQESVKRQEEA | 1 + 2 + 3 |
| | | | LRQLG (SEQ ID NO: 1) | |
| | | B | SEQERREKAKEEVRELNEEFKEAEKRFRKLQEETKKALEEVEEL | |
| | | | NQRFEEALEEVERIKRRQK (SEQ ID NO: 2) | |
| | | C | DEEEEKKKEEKKKEERLKELREEWKRLRERLQELEEQHRELQEE | |
| | | | LKELREKWKRLKERLEELREKLRREEQRRREEK (SEQ ID NO: 3) | |

By way of further example, Table 5 has the following entry, showing that polypeptides that comprise an amino acid sequence at least 70%, 75%, 80%, 85%, 90%, 91%, 92%, 93%, 94%, 95%, 96%, 97%, 98%, 99%, or 100% identical to the amino acid sequences of (a) SEQ ID NOS 54, 55, and 9; or (b) SEQ ID NOS 63, 55, and 9; or (c) SEQ ID NOS 66, 55, and 9; or (d) SEQ ID NOS 72, 55, and 9, each form an obligate heterotrimer.

| | | | | |
|---|---|---|---|---|
| DHT03_ | ABC | A | SEKEKVEELAQRIREQLPDTELAREAQELADEARKSDDSEALKVVYL | 54 + 55 + 9, |
| 1arm_ | | | ALRIVQQLPDTELAREALELAKEAVKSTDSEALKVVYLALRIVQQLP | or |
| A21/B/C | | | DTELARLALELAKKAVEMTAQEVLEIARAALKAAQAFPNTELAELML | |
| | | | RLAEVAARVMKELERNDEEIKKSDELDDESLLEDIVELLKEIIKLWK | |
| | | | ILVEVSDVMLKLIS (SEQ ID NO: 54) | |
| | | | or | |
| | | | SEKEKVEELAQRIREQLPDTELAREAQELADEARKSDDSEALKVVYL | 63, 55, and 9 |
| | | | ALRIVQQLPDTELAREALELAKEAVKSTDSEALKVVYLALRIVQQLP | |
| | | | DTELARLALELAKKAVEMTAQEVLEIAKAALKAAQAFPNTELAELML | |
| | | | RLAEVAARVMKELERNDEEIKKSDELDDESLLEDIVELLKEIIKLWK | |
| | | | ILVEVSDVMLKLIS (SEQ ID NO: 63) | |
| | | | or | |
| | | | SEKEKVEELAQRIREQLPDTELAREAQELADEARKSDDSEALKVVYL | 66, 55, and 9, |
| | | | ALRIVQQLPDTELAREALELAKEAVKSTDSEALKVVYLALRIVQQLP | or |
| | | | DTELARLALELAKKAVEMTAQEVLAIAKAALKAAQAFPNTELAELML | |
| | | | RLAEVAARVMKELERNDEEIKKSDELDDESLLEDIVELLKEIIKLWK | |
| | | | ILVEVSDVMLKLIS (SEQ ID NO: 66) | |
| | | | or | |
| | | | SEKEKVEELAQRIREQLPDTELAREAQELADEARKSDDSEALKVVYL | 72, 55, and |
| | | | ALRIVQQLPDTELAREALELAKEAVKSTDSEALKVVYLALRIVQQLP | 9 |
| | | | DTELARLALELAKKAVEMTAQEVLAIAKAALKAAQAFPNTELAELML | |
| | | | RLAEVAARVMKELERNDEEIKKSDELDDASLLADIVRLLAEIIKLWK | |
| | | | ILVEVSDVMLKLIS (SEQ ID NO: 72) | |

Other such combinations are clearly noted in the Tables, and the relevant combinations of polypeptides to form the heterotrimers, heterodimers, and heterotetramers will be clear to those of skill based on the teachings herein.

In one embodiment, heterotrimers, heterodimers, or heterotetramers comprise as interaction hub building blocks between chains in larger closed structures. As shown in the examples, the inventors demonstrate use of the heterotrimers, heterodimers or heterotetramers to produce four-chain A2B2 heterotetramers, nine-chain A3B3C3 nonamers, and twelve-chain A4B4C4 dodecamers—generated through geometry-aware rigid fusion to themselves and other designed proteins. A notable feature here is that these new assemblies can continue to be built out recursively, as they have outward facing chains with two or more free termini. The number and orientational accessibility of the chain termini found in both the base heterotrimers and the higher order assemblies enable the display of multiple distinct functional domains for signaling and other potential applications. The modularity and orthogonality of the designed protein interfaces makes it possible to combine the heterotrimer, heterodimer, or heterotetramer with other building blocks to construct more diverse nanostructures.

The disclosure further provides kits comprising one or more polypeptide, fusion protein, nucleic acid, expression vector, host cell, and/or heterotrimer of any preceding claim.

The disclosure also provides methods for use of the polypeptide, fusion protein, nucleic acid, expression vector, host cell, heterotrimer, and/or kit of any preceding claim for any suitable purpose, including but not limited to displaying antigens and administering to a subject to elicit an immune response, to act as an interaction hub building blocks between chains in larger closed structures, etc. For example the heterotrimers disclosed herein permit attachment of multiple, different monomeric proteins at each free termini (as described in the examples) and to spatially present them in a desired way.

The disclosure also provides methods for computational design of the polypeptide or fusion protein of any preceding claim, comprising any method or steps as disclosed in the examples that follow.

### EXAMPLES

### Abstract

The de novo design of three protein chains which associate to form an obligate ‘ABC’ heterotrimer, but not binary AB, AC and BC heterodimers, is an outstanding challenge for protein design. We designed helical heterotrimers with specificity conferred by buried hydrogen bond networks and large aromatic residues to enhance shape complementary packing. We obtained ten designs for which all three chains cooperatively assembled into heterotrimers with few or no other species present. Crystal structures of a helical bundle heterotrimer and extended versions, with helical repeat proteins fused to individual subunits, showed all three chains assembling in the designed orientation, We used these heterotrimers as building blocks to construct larger cyclic oligomers, which were structurally validated by electron microscopy. Our three-way junction designs provide new routes to complex protein nanostructures and enable the scaffolding of three distinct ligands for modulation of cell signaling.

### INTRODUCTION

Over half of the proteins found in the Protein Data Bank (PDB) assemble to form homo-oligomers or hetero-oligomers. The most abundant hetero-oligomers in nature are heterodimers. Heterotrimers are less widespread. ABC heterotrimers are a difficult design challenge because of the interaction cooperativity required for the three unique components to assemble only into the desired structure. Although an ABC heterotrimer only has one extra component compared to a heterodimer, the latter has only 4 alternate species (A, B, AA, BB), while an ABC heterotrimer has 15 alternate species (A, B, C, AB, AC, BC, AAA, BBB, CCC, AAB, ABB, AAC, ACC, BBC, BCC). We set out to design cooperatively assembling heterotrimers in which only the ABC species forms. We reasoned that heterotrimers could be designed by burying polar residues capable of making hydrogen bond networks in the core and by incorporating large aromatic residues for implicit negative design²² against non-ABC assemblies—such sidechains can complicate core packing in undesirable alternative states by causing steric clashes or large cavities.

### Results

### Design and Characterization of ABC Heterotrimer Left-Handed Coiled Coils

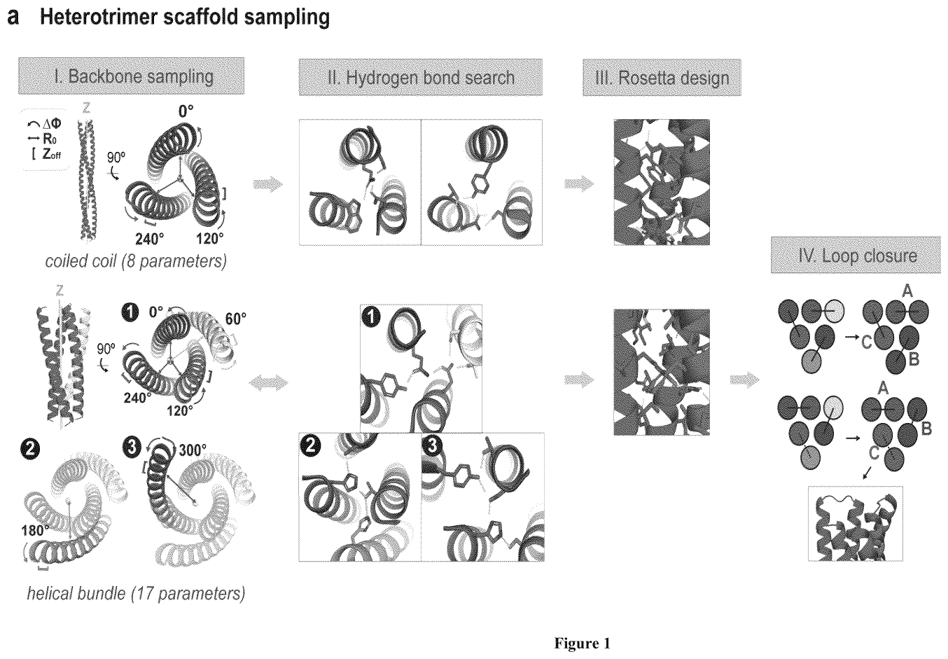

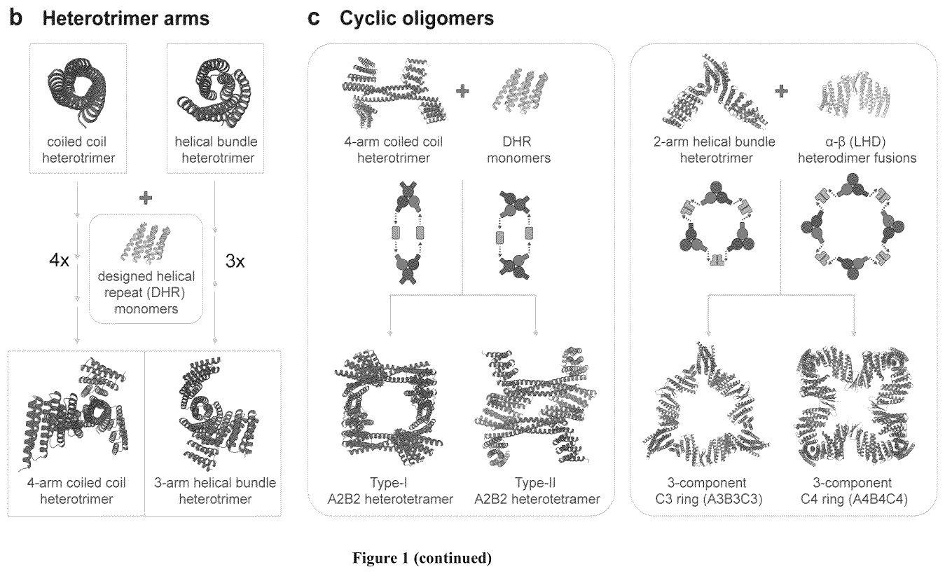

The simplest case of an ABC heterotrimer is a coiled coil, in which each chain is a single helix (FIG. 1a). A generalized Crick coiled coil parameterization^{23,24} approach was used to sample the helical phase (ΔΦ₁), supercoil radius (R), and z-offset (Z_{off}) for each helix individually. The supercoil phases (ΔΦ₀) were restricted to 0°, 120°, 240°, while the supercoil (ω₀) and helical twist (oi) were kept at ideal values −2.85 and 102.85, respectively, to generate left-handed supercoils with 3.5 residues per turn and a 7 residue (heptad) periodicity across two turns. These poly-alanine backbones were then input to Rosetta™ Monte Carlo HBNet, which places residues with polar groups across the interface such that all heavy atom donors and acceptors form hydrogen bond networks. We searched for backbones capable of hosting three hydrogen bond networks simultaneously, with each network spanning all three helices and at least two networks contributing one tyrosine or tryptophan residue each. The helices for chains B and A were then trimmed by two and four heptads, respectively, to make it easier to keep track of each chain during downstream characterization and to also allow for additional electrostatic interactions between the termini. RosettaDesign™ was then used to optimize the amino acid sequence for the remaining residues, keeping the identities and conformations of the HBNet residues fixed (we hypothesized that fully hydrophobic heptads above and below the networks would help keep the hydrogen bonding residues in place). Designs were filtered by hydrogen bond network satisfaction²⁸, packing around the networks, secondary structure shape complementarity, and 1-DDT scores from the deep learning framework DeepAccNet²⁹.

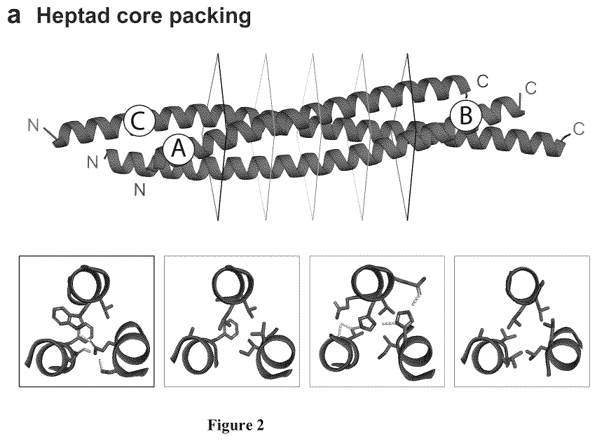

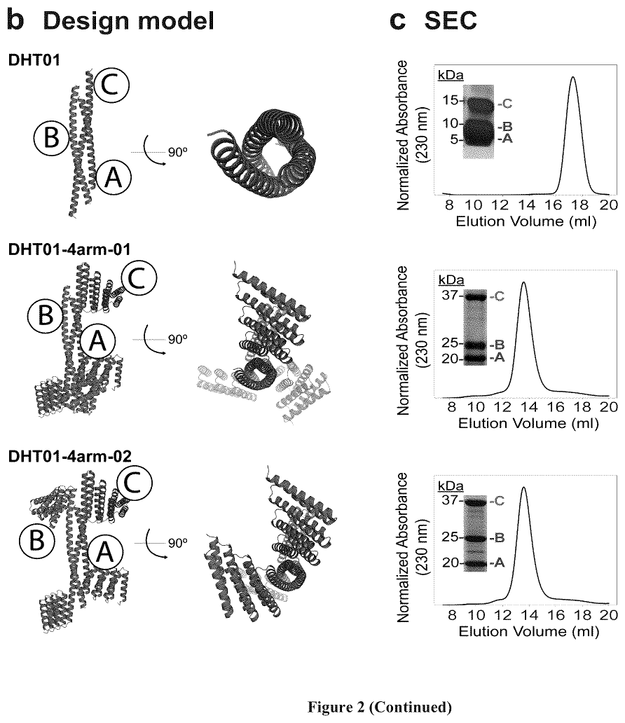

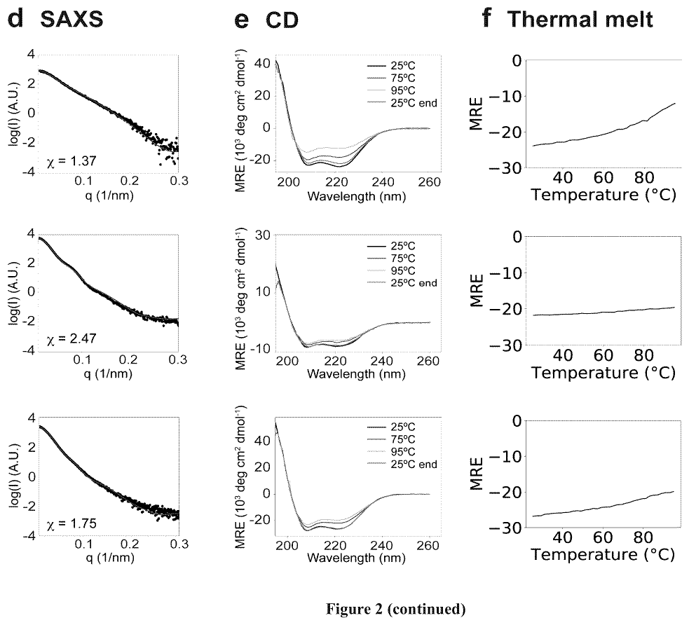

We obtained genes encoding 20 coiled coil designed heterotrimers (DHTs) in a tricistronic E. coli expression vector with one chain having a 6×His-tag and a second chain having Strep-tag II, expressed the proteins, and purified by immobilized metal affinity chromatography (IMAC) and Strep-Tactin pull-down. All designs were soluble and for 6 designs, all three components were observed by liquid chromatography-mass spectrometry (LC-MS) after both pull-down approaches. Of these, 5 eluted as monodisperse peaks by SEC, but only 1 design (DHT01) was an exclusive ABC heterotrimer by native mass spectrometry (nMS) and had good agreement with the design model via small angle X-ray scattering (SAXS) (FIG. 2d, Table 7). This design was found to be helical and thermostable up to 95° C. by circular dichroism (CD) (FIG. 2e).

TABLE 7
Summary of SAXS analysis

| | | | | | | | | Porod | | |
|---|---|---|---|---|---|---|---|---|---|---|
| | I(0) | I(0) | | | | | | volume | | |
| | (cm−1) | (cm−1) | Rg | Rg | Rg | Rc | | estimate | Dmax | |
| Design name | [from P(r)] | [Guinier] | [from P(r)] | [Guinier] | [model] | (Å) | Vr | (Å3) | (Å) | Px |
| DHT01 | 19 | 19.7 | 28.54 | 28.85 | 29.55 | 11.7 | 1.4 | 40166 | 91 | 4 |
| DHT01-4arm-01 | 39.2 | 47.5 | 35.51 | 43.14 | 43.56 | 14.8 | 2.45 | 186263 | 94 | 4 |
| DHT01-4arm-02 | 27.4 | 31.8 | 37.47 | 44.24 | 43.67 | 25.9 | 2.45 | 195793 | 97 | 3 |
| DHT02 | 172 | 172 | 22.07 | 21.8 | 18.75 | 14.7 | 1.98 | 50865 | 80 | 4 |
| DHT03 | 132 | 129 | 21.92 | 21.5 | 19.28 | 14.8 | 1.99 | 50793 | 85 | 3.8 |
| DHT04 | 232 | 238 | 21.56 | 21.57 | 18.83 | 14.9 | 1.97 | 50104 | 76 | 3.9 |
| DHT05 | 8.74 | 9.12 | 21.02 | 21.07 | 18.7 | 13.9 | 3.24 | 54102 | 65 | 3.5 |
| DHT06 | 2.14 | 2.16 | 19.66 | 18.59 | 18.83 | 13.8 | 1.58 | 40741 | 61 | 3.9 |
| DHT07 | 11.1 | 11.9 | 20.91 | 20.97 | 18.69 | 13.4 | 4.06 | 47859 | 69 | 3.4 |
| DHT08 | 4.23 | 4.32 | 21.26 | 21.32 | 18.42 | 13.2 | 4.15 | 43482 | 73 | 3.9 |
| DHT09 | 7.11 | 6.92 | 22.46 | 22.1 | 18.84 | 14.6 | 4.7 | 56189 | 79 | 3.8 |
| DHT10 | 8.55 | 9.35 | 20.7 | 21.19 | 18.12 | 13.8 | 3.93 | 45697 | 67 | 3.8 |
| DHT03_A21/B/C | 8.09 | 8.61 | 26.01 | 26.01 | 23.93 | 16.9 | 3.1 | 88917 | 85 | 3.4 |
| DHT03_A/B21/C | 5.07 | 5.07 | 27.72 | 28.95 | 24.22 | 18 | 5.94 | 99586 | 85 | 2.9 |
| DHT03_A21/B21/C | 11.9 | 11.8 | 35.76 | 36.31 | 37.31 | 24.2 | 3.45 | 131455 | 97 | 3.8 |
| long | | | | | | | | | | |
| DHT03_3arm_A21/ | 2.29 | 2.49 | 33.39 | 35.5 | 32.41 | 22.7 | 3.5 | 136860 | 93 | 2.8 |
| B21/C53 | | | | | | | | | | |
| DHT03_3arm_A21/ | 10.2 | 9.33 | 34.68 | 37 | 33.61 | 24.2 | 5.43 | 118238 | 96 | 3.5 |
| B21/C59 | | | | | | | | | | |
| DHT03_3arm_A21/ | 14.6 | 16.1 | 33.67 | 36.26 | 33.48 | 21.8 | 4.34 | 131400 | 98 | 2.9 |
| B21/C62 | | | | | | | | | | |
| DHT03_3arm_A21/ | 8.56 | 9.31 | 35.17 | 38.76 | 35.85 | 24.2 | 3.57 | 189747 | 93 | 2.6 |
| B21/C82 | | | | | | | | | | |
| DHT03_2arm_A21/ | 23 | 22 | 32.67 | 33.19 | 30.02 | 21 | 2.27 | 137036 | 97 | 3.8 |
| Bt18/C | | | | | | | | | | |
| DHT03_2arm_A21/ | 59.2 | 59.3 | 28.83 | 29.26 | 26.17 | 20.2 | 2.74 | 131987 | 91 | 3.6 |
| B14/C | | | | | | | | | | |
| DHT03_2arm_A21/ | 60.7 | 58.4 | 31.15 | 31.32 | 27.6 | 20.2 | 2.55 | 102992 | 97 | 3.9 |
| B62/C | | | | | | | | | | |
| DHT05_1arm_A62/ | 5.51 | 5.23 | 25.2 | 24.5 | 23.99 | 15.9 | 1.67 | 64714 | 93 | 3.2 |
| B/C | | | | | | | | | | |
| DHT05_1arm_A/B/ | 18 | 19.4 | 28.22 | 28.38 | 28 | 17.1 | 2.05 | 67635 | 91 | 3.9 |
| C62 | | | | | | | | | | |
| DHT05_2arm_A62/ | 35.3 | 36.4 | 31.54 | 32.56 | 31.03 | 20.7 | 3.17 | 103932 | 97 | 4 |
| B14/C | | | | | | | | | | |
| DHT05_3arm_A62/ | 25.8 | 27.9 | 35.75 | 37.94 | 34.99 | 22.3 | 2.37 | 108636 | 97 | 3.8 |
| B71/C62 | | | | | | | | | | |
| DHT05_2arm_A62/ | 28 | 30.9 | 32.71 | 35.53 | 33.02 | 18.5 | 1.92 | 106756 | 97 | 3.9 |
| B14(2)-LHD101B14/C | | | | | | | | | | |
| DHT05_2arm_A62/ | 4.86 | 6.84 | 35.39 | 47.64 | 46.9 | 18.3 | 4.82 | 148769 | 95 | 2.6 |
| B14(2)-LHD101B14 + | | | | | | | | | | |
| LHD101A53/C | | | | | | | | | | |
| C2-DHT01-01 | 82.5 | 99.7 | 39.9 | 53 | 48.51 | 41.07 | 5.41 | 354220 | 97 | 4.1 |
| C2-DHT01-02 | 114 | 142 | 44.62 | 53.5 | 48.28 | 41.46 | 5.86 | 406074 | 97 | 3.8 |
| C2-DHT01-03 | 129 | 157 | 40.85 | 51.05 | 44.52 | 33.7 | 5.27 | 360645 | 97 | 3.6 |
| C2-DHT01-04 | 92.1 | 119 | 40.81 | 52.1 | 48.92 | 36.84 | 2.86 | 373163 | 97 | 3.9 |

Rg = radius of gyration; Rc = cross-sectional radius of gyration determined from Guinier fitting; Px = Porod exponent.

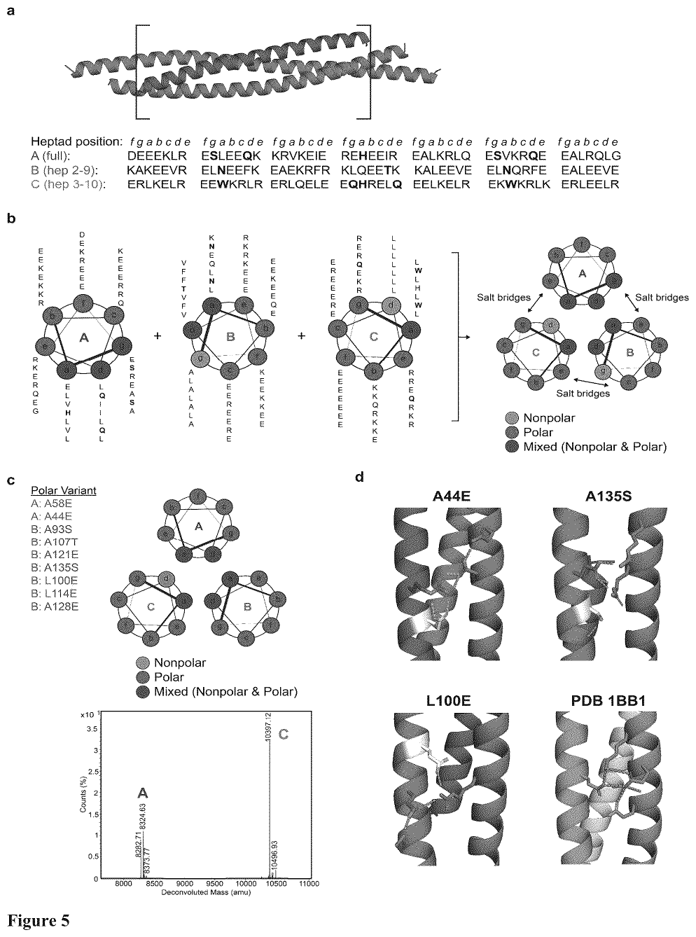

A helical wheel representation of DHT01 (FIG. 5) over the shared 7 heptad interface between all three chains shows that position ‘g’ is fully nonpolar on chain B and mixed (nonpolar and polar) on chain A, while being fully polar on chain C. This differs from a previously designed heterotrimeric coiled coil solved by x-ray crystallography, which used either glutamate or lysine at positions ‘e’ and ‘g’ to dictate specificity. To determine whether nonpolar residues are actually needed at position ‘g,’ a variant was constructed (FIG. 5) with serine, threonine, or glutamate mutations on chains A and B. In the IMAC pull-down, chain B was not present stoichiometrically, suggesting that nonpolar residues at ‘g’ are necessary for DHT01 specificity. Success in generating specificity from purely ionic interactions in the previously solved coiled coil crystal structure could be due to the much smaller size of that construct (each peptide was 4.5 heptads long) and the absence of polar residues at typical solvent exposed ‘b’ and ‘c’ positions (alanine is present instead) to reduce the likelihood of undesired competitive ionic interactions within each chain. This design principle, clearly successful at the peptide scale, could lead to solubility issues in cells and lower robustness to downstream modifications, though we have not tested this directly. In our case, DHT01 specificity is likely derived from hydrophobic and hydrogen bond network packing across ‘a,’ ‘d,’ and ‘g’ positions, and may possibly be supplemented by favorable electrostatic interactions across the three chains.

Extending an ABC Heterotrimeric Coiled Coil with Repeat Protein Arms

To determine if DHT01 could serve as an organizing hub for larger protein assemblies, monomeric designed helical repeat (DHR) proteins³⁰ were rigidly fused onto available (N and C) termini using the Rosetta™ HelixFuse protocol, with each rigid fusion being referred to as an “arm” (FIG. 1b). Repeat proteins are an attractive choice for fusion because they can be extended or contracted simply by adding or removing repeat units, and hence allow for considerable design plasticity. Genes encoding two 3-arm constructs, four 4-arm constructs, and one 5-arm construct were expressed and purified like DHT01. All designs were soluble, but only three designs had 3 equimolar components by SDS-PAGE gel in both pull-down experiments. Two 4-arm heterotrimers retained exclusive ABC heterotrimer assembly via SEC, nMS, SAXS, and CD; the armed constructs are more thermostable than the original coiled coil heterotrimer at 95° C. (FIG. 2b-f). The ability of the ABC heterotrimer to support rigid repeat protein fusions suggests that the base coiled coil construct folds as designed and provides new connections points for downstream nanomaterials design.

### Design and Characterization of ABC Heterotrimer Helical Bundles and Co-Expressed Arms

To explore the ability to design heterotrimers when larger interfaces are available for installing hydrogen bond networks, we extended our computational approach to helical hairpin units. We experimented with two approaches: first, sampling superhelical parameters for all six helices at once (see Methods), and second, making the search more tractable by first sampling parameters for 4 of the helices, filtering, and then adding on the two remaining helices (FIG. 1a). Here, the supercoil radius (R), helical phase, and z-offset (Z_{off}) were sampled first for only the three inner helices (at superhelical phases 0°, 120°, 240°) and one outer helix (at 60°), and then Monte Carlo HBNet^{25,26} was used to search for a 4-helix network with at least one tyrosine or tryptophan. For backbones that passed this criteria, the supercoil radius (R), helical phase (ΔΦ₁), and z-offset (Z_{off}) of an additional fifth helix placed at supercoil phase 1800 were sampled, and Monte Carlo HBNet was used to search for hydrogen bonds involving this new helix and 3 of the already placed inner helices. Subsequently, the sixth helix was added at supercoil phase 3000 and Monte Carlo HBNet was again used to search for hydrogen bond networks spanning the first 3 inner helices and the new 6th helix (FIG. 1a). Rosetta™ combinatorial sequence design calculations were carried out on the resulting helical bundle backbones, keeping the HBNet residues fixed as described above for the coiled coil heterotrimers. Designs with exposed hydrophobic patches were removed using the Rosetta™-integrated Developability Index SAP filter to prevent sticky mis-assemblies and aggregation. To create the ABC heterotrimer, an inner and outer helix were connected with a short loop in either a clockwise or counterclockwise orientation, with loops all on the same side or on opposite ends of the heterotrimer (see Methods).

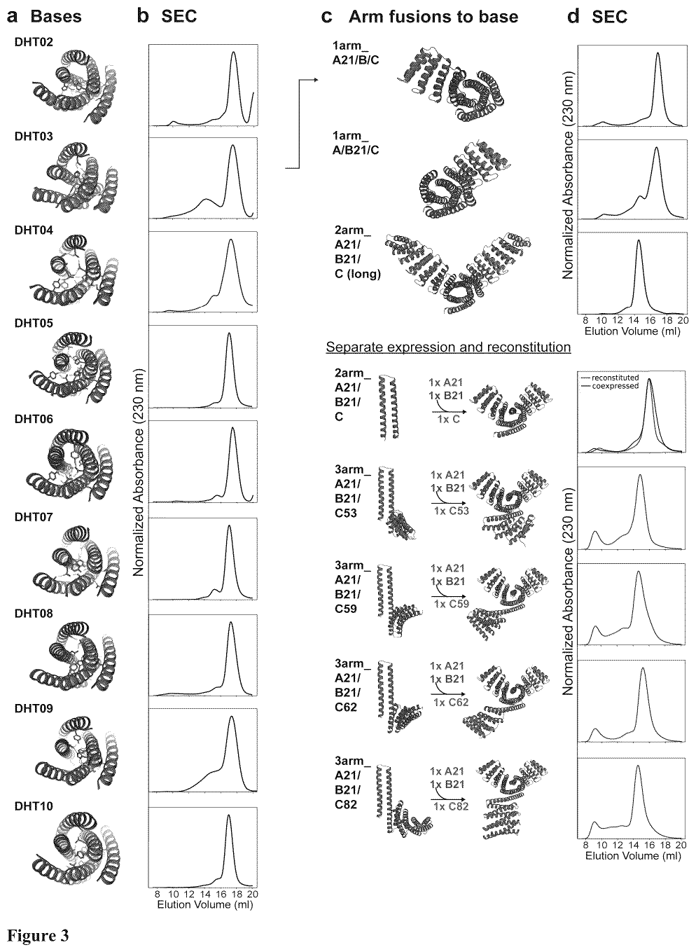

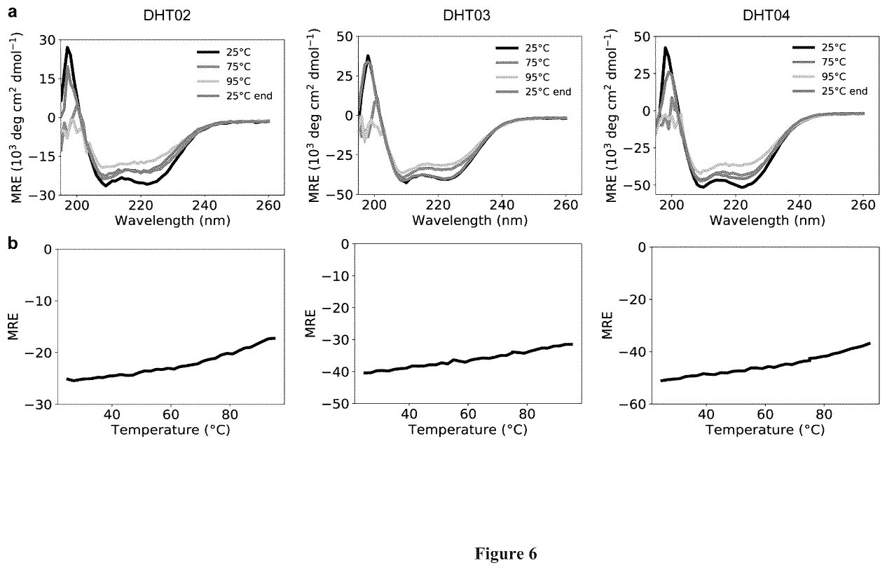

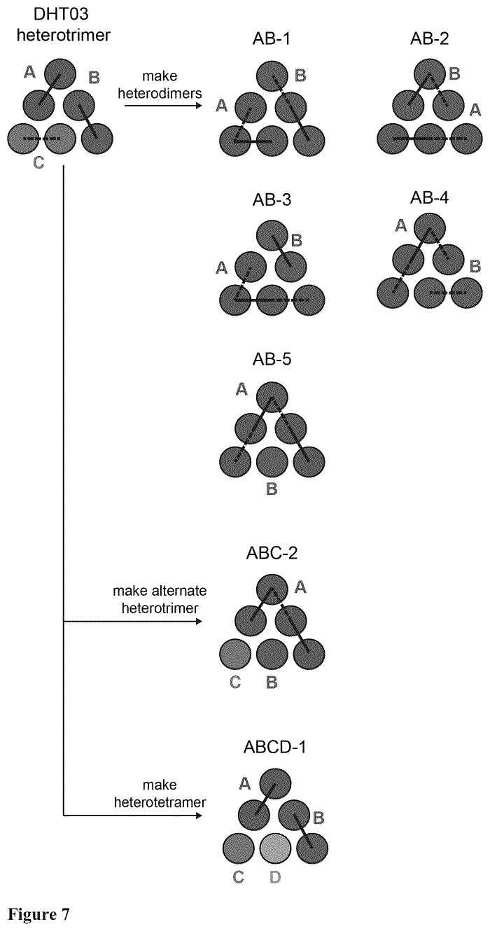

Genes encoding 85 heterotrimers in a tricistronic expression vector were obtained, and the proteins were expressed in E. coli and purified via IMAC with only one chain having the 6×His-tag. Nine of the designs (FIG. 3a) had monodisperse SEC peaks (FIG. 3b), were almost exclusively ABC by nMS, and had good SAXS fits to the design models (Table 7). CD measurements for DHT02-04 showed they were helical and thermostable as expected (FIG. 6). Eight of the designs are parallel heterotrimers, while DHTO9 is an antiparallel heterotrimer. From this set tested, 12 other designs showed the presence of 3 components by LC-MS but showed heterogeneity (ABC+other alternate species) in nMS. For 3 other designs, only the ABC heterotrimer was determined to form by nMS (when the respective SEC fraction was analyzed) but the heterotrimer SEC peak was preceded by soluble aggregate that would make building with these constructs difficult downstream. We explored the dependence of cooperativity on connectivity between the three chains by changing the positions of the loops across different helices in DHT03, while keeping the rest of the interface intact. We found that 5 heterodimers, 1 alternate ABC heterotrimer, and 1 ABCD heterotetramer could be made to assemble specifically, as determined by nMS (FIG. 7). The ability to sustain alternate loop closures increases the number of heteromeric building blocks available for future nanostructure assembly applications and may enable rigid fusion of functional domains to the different termini available.

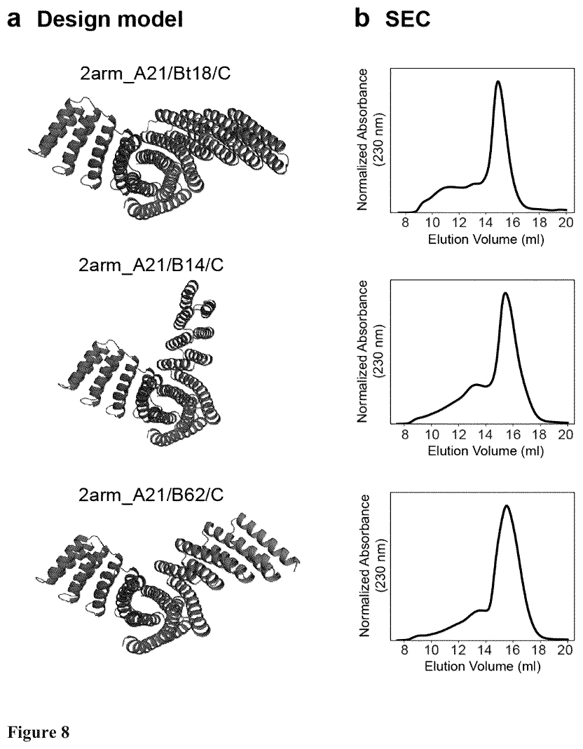

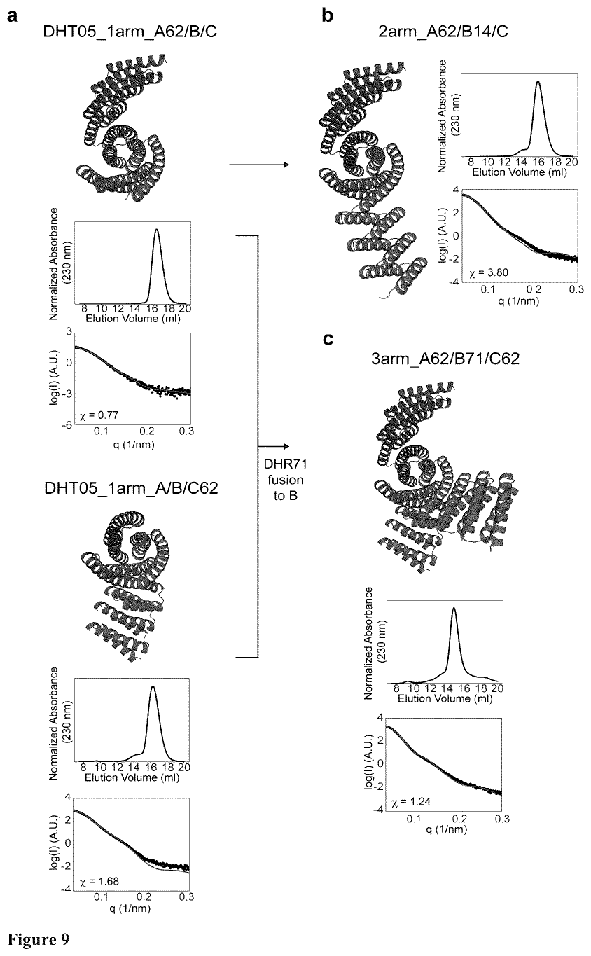

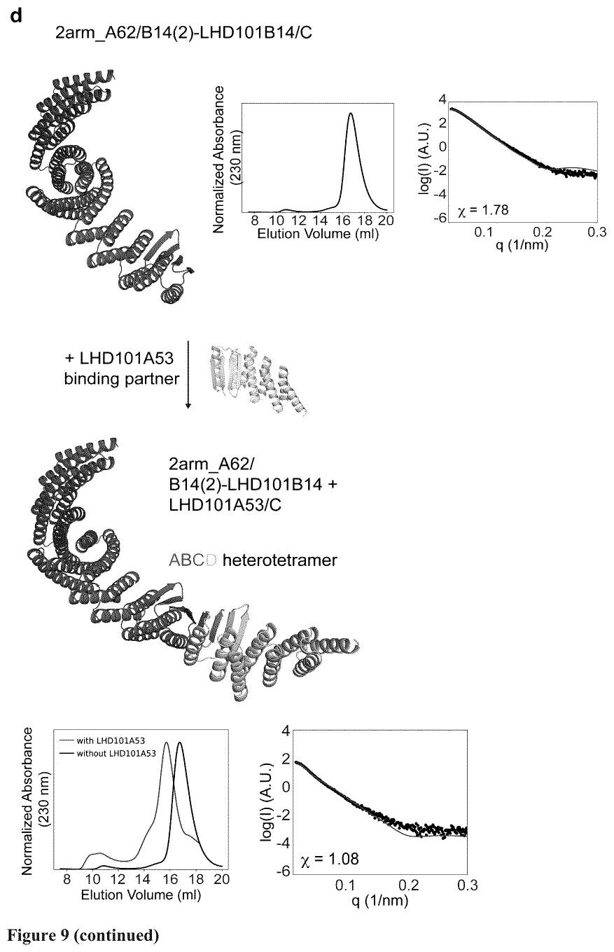

We decided to test rigid fusions sequentially to evaluate the effect of each fusion independently on ABC heterotrimer formation. Using this strategy, we found that of the first four helical bundle heterotrimers shown in FIG. 3, two were able to sustain 3-arm extensions. We illustrate this with DHT03 in FIG. 3c, d and in FIG. 8; fusion data for DHT05 is in FIG. 9. Ten 1-arm fusions to DHT03 were tested via tricistronic expression, with two eluting with monodisperse peaks by SEC and forming exclusive ABC heterotrimers by nMS (FIG. 3d rows 1 and 2). Subsequent 2-arm fusions were made by combining the two working 1-arm fusions and testing 9 more new fusions to chain B while keeping chain A constant. Eight out of 10 designs tested had 3 equimolar bands by gel, 6 designs had a distinct heterotrimer SEC peak present with the ABC species detected by nMS, and 4 had a very good fit to the design model via SAXS (FIG. 3d row 3; FIG. 8).

### Heterotrimer Arms can be Separately Expressed and Reconstituted to Form the ABC Species

To facilitate downstream higher-order assembly design, we investigated whether the ABC heterotrimer could be reconstituted from separately expressed individual chains. Because of the hydrophobic nature of the core, each chain of the heterotrimer can self-associate when expressed separately. We reasoned that the designed ABC heterotrimer state would likely be lower in free energy than possible off-target homo-oligomeric species, and hence that heat annealing could promote assembly to the design target state. To test this idea, an equimolar amount of individually purified A, B, and C of DHT03_2arm_A21/B21/C were mixed together and run through an annealing protocol (see Methods) to allow these interfaces to reassemble in the presence of all components. A monodisperse SEC peak was observed belonging to the ABC heterotrimer state as confirmed by overlap with its SEC co-expressed tricistronic version (FIG. 3d). To explore the use of this approach for building, four different repeat protein fusions to chain C were annealed in the same manner and all four 3-arm heterotrimers were successfully reconstituted. Although co-expression was used successfully for the characterization described above, it has the drawback that the expression levels of each chain cannot be precisely controlled and therefore imbalances could potentially inhibit proper assembly. The successful reconstitution of these ABC assemblies, following the independent expression and purification of each chain and explicit mixing at a 1:1:1 ratio, enables their use in conjunction with other de novo proteins to mediate the specific assembly of multi-component assemblies. Independent expression also has considerable advantages over tricistronic expression from a gene synthesis perspective as DNA becomes much more difficult and expensive to synthesize with increasing construct size.

To test whether the DHT03 components could properly assemble in the presence of other helical hairpin containing chains, as would be the case for more complex assemblies, we chose DHD131, a designed helical hairpin unit heterodimer containing buried hydrogen bond networks¹². We found that our separate expression and reconstitution approach via annealing succeeded with this design: chains A and B of DHD131 could be separately expressed and purified, and when the proteins were mixed together at an equimolar ratio and annealed, a monodisperse heterodimer peak was observed by SEC. We evaluated simultaneous reconstitution of DHD131 and DHT03_3arm_A21/B21/C82, since the size difference between the two constructs (if correctly assembled) should be detected by SEC. We separately expressed and individually purified the two chains of the heterodimer and the three chains of the heterotrimer, mixed them at a 1:1:1:1:1 ratio, and carried out annealing and SEC as described above. The two major species were found to be the DHD131 heterodimer and the DHT03 heterotrimer by nMS. Some DHD131 BB homodimer was also detected, but this was observed in previous nMS analyses of the heterodimer alone¹². Thus, the heterotrimer chains can come together to form the intended ABC species even in the presence of potentially confounding additional helical hairpins. The ability to simultaneously assemble multiple hetero-oligomers from individual chains without interference opens the door to construction of diverse nanostructures with distinct multichain hubs.

### X-Ray Crystal Structures of DHT03

We succeeded in solving three high-resolution structures for DHT03: the original base construct, a 1-arm version (1arm_A21/B/C), and an elongated 2-arm version (2arm_A21/B21/C long). The first crystal structure was solved at 2.35 Å resolution. The design model, shown in darker colors, has an overall 2 Å Cα RMSD agreement to the crystal structure, shown in lighter colors, with the largest deviations upon individual chain superposition in chain C. Many of the hydrogen bonds in the design model are not present in the crystal structure, but the overall placement of these residues is relatively close in space to the design model and the ABC heterotrimer still assembles with buried polar groups in the core (water molecules were not detected). The placement of these residues also appears to be effective in specifying orientation as the chain C inversion in the design model matches that of the crystal structure. There are two possible explanations for the deviations in the hydrogen bonding between crystal structure and design model. The first is that optimization of non-polar packing in the actual structure distorts the protein slightly so that many of the designed hydrogen bonds do not form. In support of this, the crystal structure has lower Rosetta™ computed energy than the design model due to improvements in sidechain rotamer preferences, Lennard-Jones (LJ)/van der Waals interactions, and solvation energies. On the other hand, Rosetta™ does not accurately capture the high energetic cost of buried unsatisfied hydrogen bonds; and so the second possibility is that in solution or neighboring low energy states, small backbone adjustments allow for more or all of the designed hydrogen bonds to form. In any event, a lesson from this structure is that more extensive sampling around the designed conformation in the design process would be useful to determine whether nonpolar packing and hydrogen bond networks favor the same state; because of the short range nature of the hydrogen bond and the strong orientational constraints, even small distortions away from the design model can disrupt hydrogen bond networks.

The crystal structures of the one arm (biophysical data in FIG. 3 c, d row 1) and elongated two arm (biophysical data in FIG. 3 c, d row 3) fusions were both solved at 3.35 Å resolution. Over the core heterotrimer in both structures, the backbones are very similar to that of the base heterotrimer crystal structure, but the resolution is not high enough to determine the state of the designed hydrogen bond networks. The designed junctions between the repeat protein arms and the core bundle are recapitulated in the crystal structures, and hold the repeat arms rigidly close to the designed orientation. Deviations between the crystal structure and design model in the repeat protein arms increase with increasing distance from the central bundle due to lever arm effects arising from the compounding of small orientational differences in the structures of the individual repeats³⁰. These structures demonstrate that the designed heterotrimers can serve as rigid interaction hubs in asymmetric assemblies, which have been more difficult to design than symmetric ones.

We were not able to crystallize the remaining nine base heterotrimers, and turned to protein structure prediction to supplement the SEC, nMS, and SAXS data presented above. For DHT03, 4 out of 5 AlphaFold-Multimer models generated heterotrimeric structures similar in overall topology; interestingly the predicted structures are closer to the Rosetta™ design model than the crystal structure when aligned across all Cα. For eight of the other nine heterotrimer designs, AlphaFold-Multimer^{34,35} models were within 2 Å Cα RMSD of the design models; we note that physically based Rosetta™ and AlphaFold should be largely orthogonal so this level of agreement can be considered independent validation). While not as definitive as a crystal structure, the combination of structure prediction and biophysical data presented here strongly support the design models.

Core Residues with Buried Polar Groups are Essential for Mediating ABC Specificity

To better understand the importance of the buried polar groups for exclusive ABC heterotrimer assembly in DHT03, the residues intended to form hydrogen bond networks were systematically replaced. Starting from the crystal structure, residues involved in each network were repacked with non-polar residues using Rosetta™, while keeping the remaining residues intact. We refer to these as “sub_net1,” “sub_net2,” and “sub_net3,” while the combination of all these substitutions was called “sub_netall.” These four constructs were purified via the same IMAC pull-down approach as the parent heterotrimer design. In all four cases, the A, B, and C components were present in the eluate by LC-MS but the SEC spectra had broad diffuse peaks suggesting heterogeneity in the assemblies. The broadest SEC trace was observed for “sub_netall” with the entirely hydrophobic core. This hydrogen bond network replacement with nonpolar residues experiment suggests that the buried polar residues contribute to structural specificity.

### Using ABC Heterotrimer Arms to Build Multi-Component Cyclic Assemblies

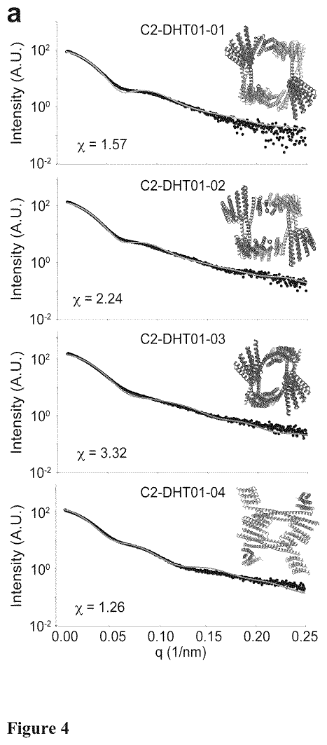

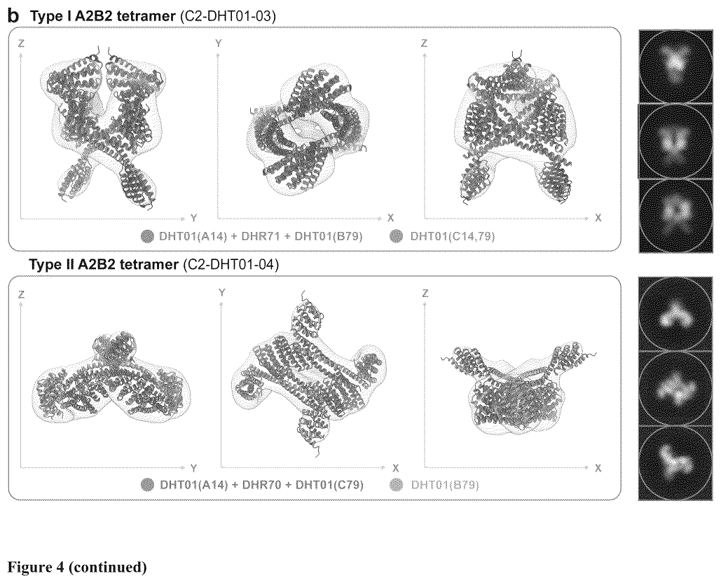

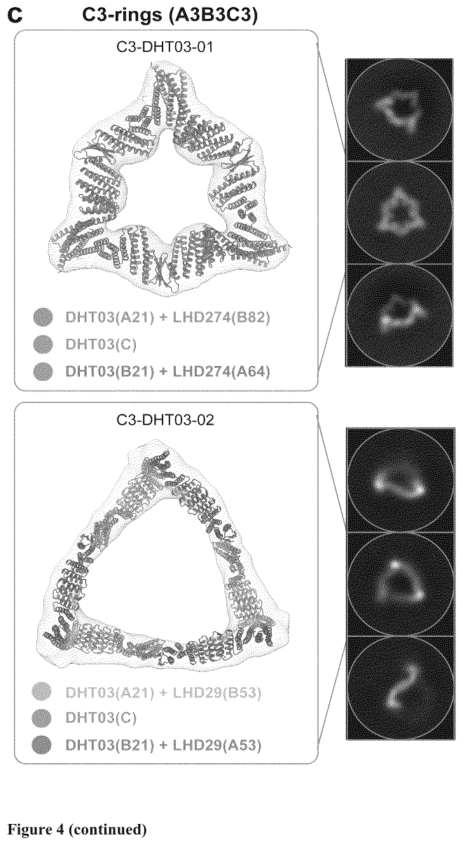

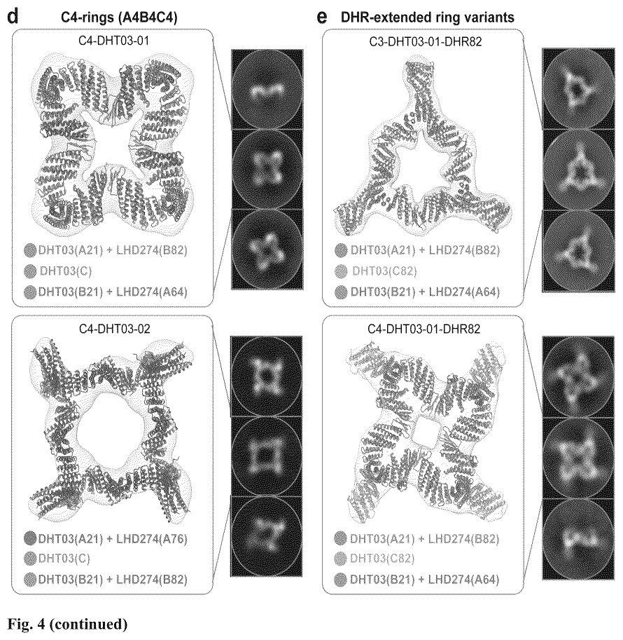

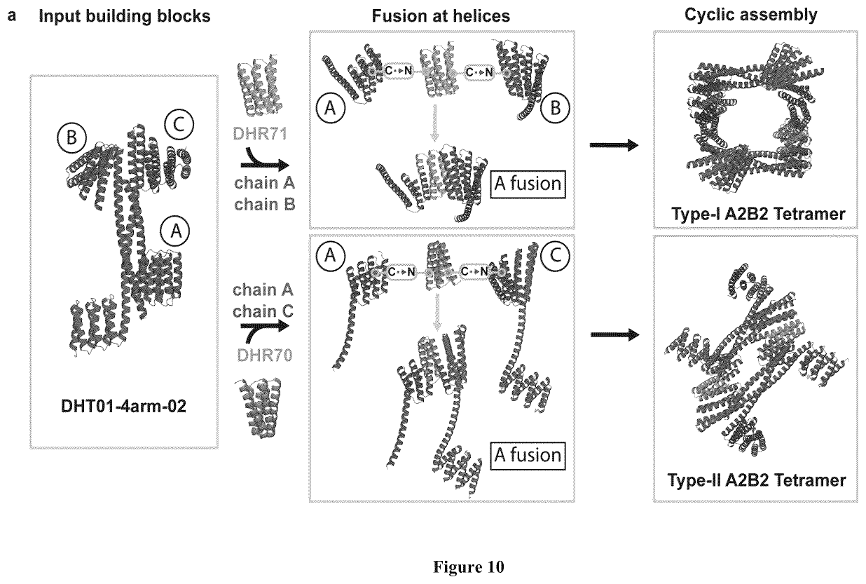

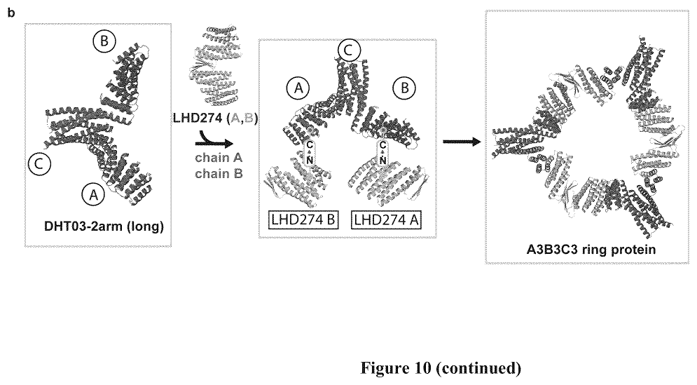

To investigate the potential of the ABC heterotrimers to serve as multichain connection hubs in larger designed nanostructures, we employed the WORMS^{36,31} software which searches over very large numbers of possible rigid fusion between building blocks to build up user-specified architectures. In a first round of architecture design, the chains of the 4-arm coiled coil ABC heterotrimers from FIG. 2 were fused with a library of designed helical repeat proteins to generate closed rings with different cyclic symmetries. As illustrated in FIG. 1c, there are two ways to connect two of DHT01-4arm-02's three chains to form closed cycles: (1) fusion between the single DHR-arms on chains A and B, or (2) fusion between chain A's DHR-arm and one of chain C's DHRs; these generate “type1” and “type2” closed assemblies, respectively. The placement of the original heterotrimer chains in these assemblies is indicated schematically in FIG. 1c and FIG. 10a. Twelve designs were experimentally tested via bicistronic expression. Of these, four A2B2 heterotetramers had good agreement via SAXS (FIG. 4a, Table 7) and rings were evident in negative stain (ns) EM, with 3D reconstructions obtained for two of the constructs (FIG. 4b).

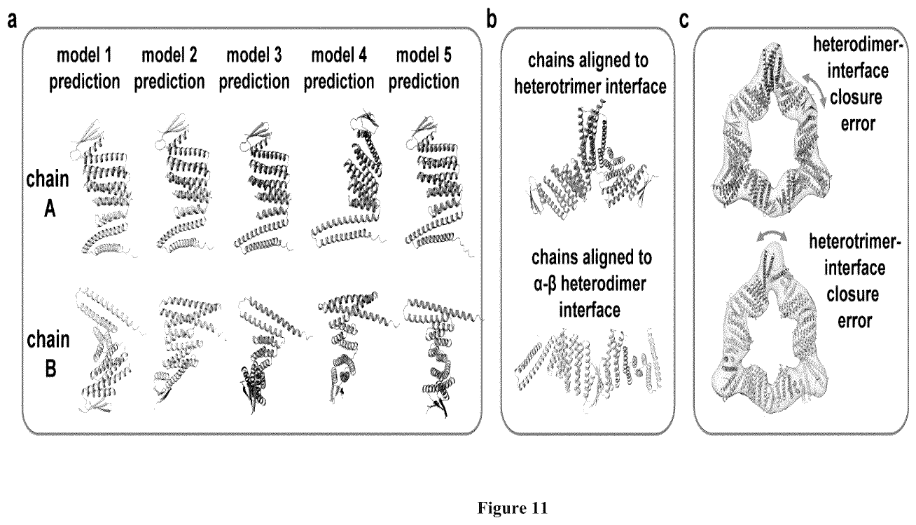

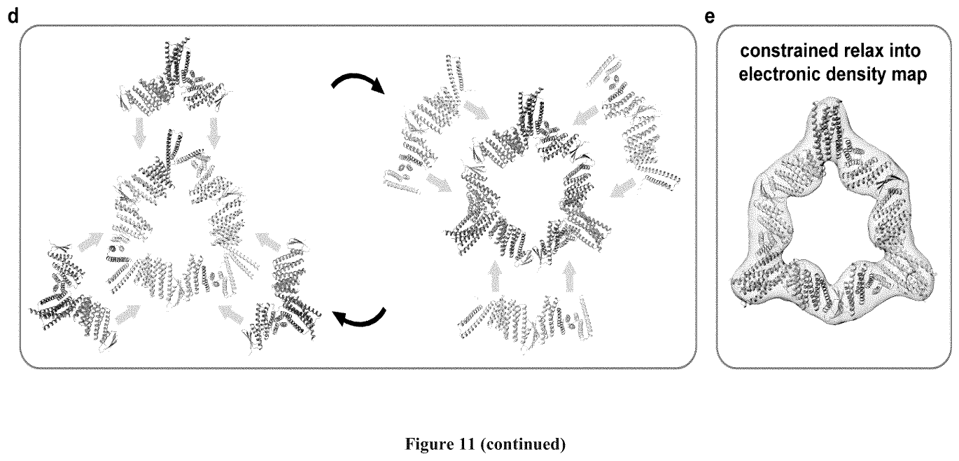

To expand the range of geometries, and to enable nanostructure assembly from individual components, we sought to design a second round of cyclic structures using the propagated 2-arm ABC heterotrimer crystal structure together with alpha/beta (LHD) heterodimers³⁷, as shown schematically in FIG. 1c and FIG. 10b. This combination of two different types of de novo hetero-oligomers in one closed structure is challenging because even slight changes in the angles of newly designed fusion points can have a significant impact in resulting geometries. Genes were obtained for five A3B3C3, two A4B4C4, and three A5B5C5 rings. Ring structures were observed for six designs by nsEM; two designs displayed a mixture of C3 and C4-symmetric states, but the remaining four samples (two A3B3C3 and two A4B4C4 rings) were homogeneous. 3D reconstructions for three of these four rings revealed cyclic symmetry one degree lower than the computational designs (C3-DHT03-02 formed an A3B3C3 ring as intended). Assembly of smaller rings results in less loss of translational entropy—this likely results from small inaccuracies in the design models of the components lacking crystal structures and internal flexibility of the repeat protein connectors. Regenerating the design models using the experimentally observed symmetries (FIG. 11) yielded structural models that closely fit the electronic density maps (FIG. 4c, d).

As is evident from the images in FIGS. 4c and 4d, the cyclic structures retain one of the original heterotrimer helical hairpins in each corner; these short, outward facing protein chains provide fusion points for further elaboration of higher order symmetric assemblies. To evaluate the potential for building up larger nanostructures using the rings as hubs, and explore the modularity of the designed building blocks, we replaced the original outward facing chain C helical hairpin with the chain C DHR82 fusion (as it was the largest 4-repeat DHR) from FIG. 3d, row 8. This was done for two designs (C3-DHT03-01 and C4-DHT03-01), with chain C replaced by fusion C82 through expressing and purifying the three components separately, mixing them in equal molar quantities, and reconstituting using heat annealing. The primary-peak fractions from SEC were collected and inspected by nsEM, and rings with DHR82-extended arms were observed for both C3-DHT03-01 and C4-DHT03-01. The micrographs displayed clear arms extending from the rings and were used to reconstruct 3D electronic density maps (FIG. 4e), indicating successful assembly of symmetric complexes with branching capabilities at this terminus.

### DISCUSSION

We have demonstrated that computational design can be used to create cooperative ABC heterotrimers that can assemble as free standing helical units or as hubs in larger designed assemblies. Their small base size and high soluble expression make them useful for biological scaffolding applications involving the recruitment or display of three different proteins. The ability to sustain rigid helical fusions to monomeric repeat proteins enables the incorporation of arms that can be extended to provide three new elongated connection points. We show that the heterotrimers can be used as interaction hub building blocks between chains in larger closed structures—four-chain A2B2 heterotetramers, nine-chain A3B3C3 nonamers, and twelve-chain A4B4C4 dodecamers—generated through geometry-aware rigid fusion to themselves and other designed proteins. A notable feature here is that these new assemblies can continue to be built out recursively, unlike previously designed rings, as they have outward facing chains with two or more free termini. The number and orientational accessibility of the chain termini found in both the base heterotrimers and the higher order assemblies enable the display of multiple distinct functional domains for signaling and other potential applications. As illustrated by the larger ring designs, the modularity and orthogonality of the designed protein interfaces makes it possible to combine the heterotrimers with other heteromeric building blocks to construct more diverse nanostructures.

Our crystal structures together with the mutational data pose a fascinating fundamental biophysics puzzle. On the one hand, the crystal structure of DHT03 shows that many of the designed hydrogen bond network residues are not making hydrogen bonds due to small local distortions in the structure; instead they are buried without making any hydrogen bonds, which is expected to be extremely destabilizing. On the other hand, while DHT03 assembles exclusively to the designed heterotrimer state, mutants in which the hydrogen bond networks have been substituted by nonpolar residues appear to adopt a range of alternative states, suggesting that the hydrogen bond networks are playing an important role in conferring structural specificity as in our design conception. It is possible that these residues confer specificity even without making hydrogen bonds as alternative states could have still higher energies when they are present; or there may be very similar states populated in solution in which the hydrogen bonds are formed which favor the designed assembly.

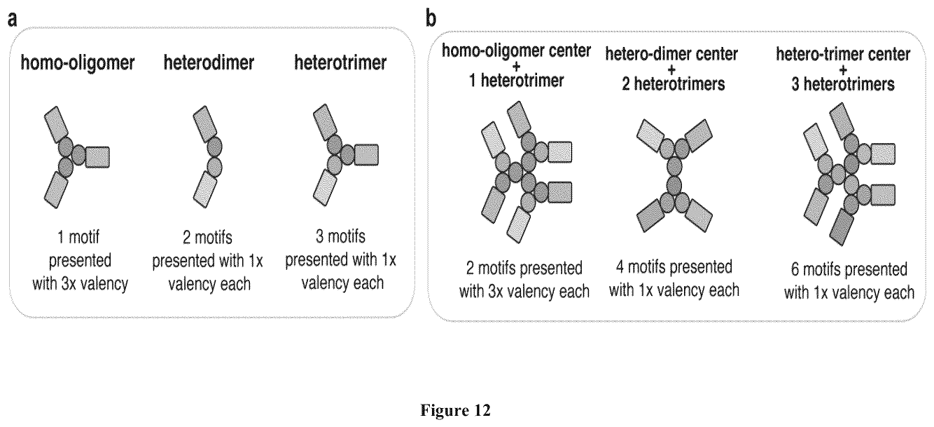

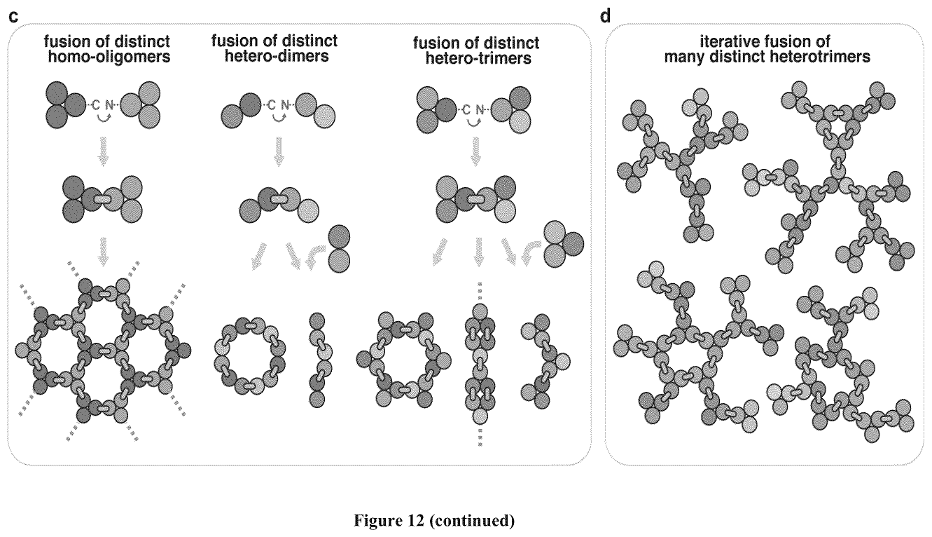

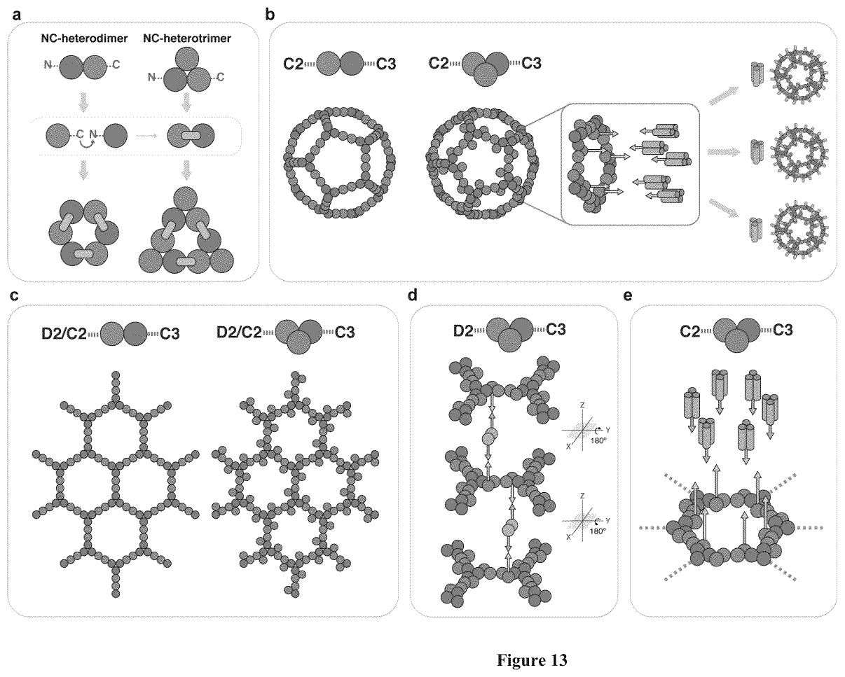

With modular heterotrimeric building blocks such as those developed in this paper, a much wider range of asymmetric assemblies become accessible, as each additional heterotrimeric interface introduces new centers for asymmetric branching (FIG. 12). Furthermore, design applications for assemblies built from components with cyclic symmetry can be limited by the small number of unique accessible termini for functionalization; whereas as illustrated by the cyclic rings designed here, use of heterotrimeric building blocks will generally result in a fusion-accessible chain at the heterotrimeric interface, providing modular design opportunities to build upon a common base scaffold (FIG. 13). More generally, these heterotrimeric interaction hubs are attractive for nanomaterial applications that require symmetry breaking to generate more sophisticated protein assemblies (as the addition of asymmetric units, like the heterotrimers, enables the construction of larger and more diverse higher-order assemblies). In addition, the heterotrimer scaffolds, its extensions, and the heteropolymers can be used as anchors to attach other small naturally occurring or de novo proteins, display these fused proteins, and recruit a response depending on the application: (1) different antigens can be displayed on each monomeric unit of the heteropolymers in order to determine if the scaffold can engage in an immune response and produce neutralizing antibodies, (2) antigen-binding fragments of an antibody (or part, or all of an antibody, pending sterics) can be fused to each monomeric unit of the heteropolymers in an effort to bind to specific antigens and determine if the appropriate T cell response can be initiated, or (3) generally proteins with binding pockets, proteins that can bind to ligands, or proteins with oligomer affinity can be fused to each monomer of the heteropolymers to determine if (assuming the proteins are placed at an appropriate distance) they are able to still function in the same manner pre-fusion, thereby enabling a better understanding of protein-protein interactions (PPI).

### Methods

### Computational Methods

### Backbone Sampling

For single helix heterotrimers, three helices were fixed at supercoil phases 0°, 120°, and 240° to generate chains A, B, and C respectively. Helix termini were kept in the same direction for a parallel orientation, while the third helix at supercoil phase 240° was inverted for an antiparallel orientation. To create a left handed coiled coil, the supercoil and helical twist were kept at ideal values −2.85 and 102.85, respectively. The helical phase (ΔΦ₁) was sampled from −100° to 100° with an interval of 20°. The three helices were sampled at a 6.5-7.5 Å distance (R) from the z-axis with an interval of 0.25 Å. Z-offset was also sampled, kept at 0 for the first helix but then sampled at −1.5, 0, and 1.5 for the second and third helices each to account for rise per residue. All helices were sampled independently across all parameters. Each helix was 77 residues in length.

For the 6-helix heterotrimers in the first sampling approach, the supercoil radius (R) and helical phase (ΔΦ₁) were sampled independently for parallel backbones, with supercoil phases fixed at 0°, 120°, and 240° for the first three inner helices and 60°, 180°, and 300° for the remaining three outer helices. If the same parameters from the coiled coil search were applied here for all six helices, more than hundreds of billions of backbones would need to be sampled simultaneously. So instead, helical phase (ΔΦ₁) was sampled from 0° to 100° with an interval of 20° and supercoil radius (R) was sampled at a 6.5-7.5 Å distance (R) with a 0.5 Å interval for the inner three helices and at 12.5-13.5 Å with a 0.5 Å interval for the outer three helices.

In the second approach, only the first three inner helices and one outer helix were sampled in the first round. The helical phase (ΔΦ₁) was sampled from −100° to 100° with an interval of 20°. The 3 inner helices were sampled at a 6.5-7.25 Å distance (R) from the Z-axis with an interval of 0.375 Å. The fourth outer helix was sampled at a 12.25-13.25 Å distance with an interval of 0.5 Å. Z-offset was kept at 0 for the first helix and sampled at −1.5, 0, 1.5 for the second, third, and fourth helices. The 5th and 6th helices were then each individually sampled across the same 3 parameters as the 4th helix. Helix termini were kept in the same direction for a parallel orientation, while the third helix at supercoil phase 240° and the sixth helix at supercoil phase 300° were inverted for an antiparallel orientation. Each helix was 35 residues in length.

### Design of Hydrogen Bond Networks

All polar residues and acidic charged (ASP/GLU) residues were considered during the search. A total of 100,000 Monte Carlo trials were attempted with extra rotamers parsed through to help increase sampling. A minimum of two TRP/TYR were required to be parts of the networks. For single helix backbones, hydrogen bond networks were searched across every other heptad such that a final core N—P—N—P—N—P—N—P—N heptad search pattern would result (N=nonpolar, P=polar). Networks were required to span all three helices and consist of at least three residues, with a total of three networks across the heterotrimer.

For 6-helix backbones resulting from the first parametric sampling approach, hydrogen bond networks were searched across the three middle heptads. Networks were required to span 5 or 6 helices, consisting of at least six residues, with at least two networks total. We required that each network contain at least one tyrosine, tryptophan, aspartate, or glutamate. Overall, MC HBNet search was slower due to the increased search space across the middle heptads at all helices, few fully satisfied long networks were found meeting our requirements, and ultimately Rosetta™ design packing around 6-10 residue networks (compared to 3-4 residue coiled coil networks) was harder, which led us to focus on the second sampling approach.

For 6-helix backbones resulting from the second sampling approach, three hydrogen bond networks were searched for such that all networks span across the three inner helices and the newly built outer helix. This would yield a core N—P-P-P—N heptad search pattern, in which every helix contributes at least 1 residue to a hydrogen bond network. We hypothesized that fully hydrophobic heptads above and below the networks would help keep the hydrogen bonding residues in place.

### Rosetta™ Design

Chains B and A for the coiled coil backbone were trimmed by two and four heptads, respectively, resulting in chain A being 49 residues, chain B being 63 residues, and chain C being 77 residues long. Two helices of the 6-helix backbone (which would ultimately constitute one helical hairpin) were optionally trimmed by one heptad. Both sets of heterotrimer bases underwent packing using RosettaDesign™, with six-helix backbones having an additional SAP mover and filter. Constraints on hydrogen bond network residues were placed. The backbones were divided up by layers (core, boundary, surface) with two total packing rounds. A scoring term was also used to enforce at least two phenylalanine at the core. A round of Fast Design calling a Monte Carlo mover was applied to enhance secondary structure shape complementarity, along with an upweighted short range hydrogen bond scoring term to maintain proper helical formation. A final minimization and repack of the sidechain rotamers was allowed after removing constraints on network residues.

### Loops to Make Helical Bundle Heterotrimers

Six-helix heterotrimers are “closed” into three helical hairpin chains in either a clockwise (A-D; B-E; C-F) or counterclockwise (A-F; B-E; C-D) orientation. Short 2-5 residue loops were generated in Rosetta™ with favorable ABEGO types. Loops were built from either available termini on each chain, with an option to delete up to 3 residues or to add 2 more residues to the existing termini to build off of as a starting point. Loops were minimized and filtered by low fragment RMSD and psipred.

### Rigid Arm Fusions to DHRs

Rosetta™ Helixfuse was used to rigidly join a library of DHRs to heterotrimer bases by joining the termini of both constructs based on secondary structure overlap; up to a heptad on the heterotrimer was allowed to be deleted, while up to a full single repeat was permitted to be deleted for the DHR. The lowest scoring rmsd overlap was accepted. A filter was subsequently applied to check for clashes between the two joined proteins to determine residues that needed to be redesigned; RosettaDesign™ was used to find optimal residues for the new helix. The best scoring fusions according to 1-DDT²⁹ and after manual inspection were ordered. For DHT01 fusions, solutions were found at only 5 of the 6 available termini.

### Design of Cyclic Rings

The generation of cyclic assemblies using WORMS fusion was performed following protocols presented in previous literature^{31,36}. Input building blocks for components (heterotrimers, heterodimers, and monomeric DHRs) were curated in a WORMS database file, wherein each entry included specification of scaffold class, pdb file path, and the range of helical residues up or downstream from N and C termini that were accessible as splice sites.

Within the WORMS software, cyclic symmetry protocols were performed (C2, C3, C4, and C5), such that closure between cyclically propagated copies of the input building blocks could be found by coordinate-alignment between residues within the specified fusion-accessible helices from each splice partner.

For rings made from DHT01, the rigidly fused DHR-arms were joined at N- and C-terminal helices, and an additional DHR repeat motif was used to brige the two heterotrimer chains. For rings made from DHT03, the rigidly fused DHR arms both possessed N-terminal helices, necessitating their fusion to alpha-beta heterodimers that possessed two C-terminal DHR arms in order to close the cyclic geometry.

The outputs from the WORMS algorithm were filtered by three criteria: sequence length, internal clashing, and ring-closure error; these values for each WORMS output were presented in score files, under the fields ‘chain_len’, ‘score0’, and ‘close_err”, respectively. The selected designs were then passed through rigid backbone sequence design using RosettaDesign™, in order to optimize the local sequence around the newly formed helical junctions. The modified positions to be designed were assigned as residues that either gained or lost contacts with neighboring residues following helical fusion.

The sequences of individual chains for these designed complexes were submitted to AlphaFold2™ for monomeric structure prediction. Complexes where the designed models for each constituent chain possessed low RMSD when aligned to AlphaFold2™ structure predictions (prioritizing alignment to predicted models with high pLDDT scores) were selected to order.

### Gene Preparation

Genes were codon optimized for bacterial expression and ordered in pET29b+ vector between the NdeI and XhoI restriction sites, with a T7 promoter and Kanamycin resistance gene. DHT02, 03, 04, and 05 had an additional de novo EHEE protein³⁹ added to the N-term of the first chain to increase molecular weight for SDS-PAGE differentiation. Constructs for co-expression were ordered using ribosome binding sites (RBS), TAAGAAGGAGATATCATCATG (SEQ ID NO: 121) and/or TAAAGAAGGAGATATCATATG (SEQ ID NO: 122), in between the chains. The last chain in base and arm sequences had a cleavable N-term 6×His-tag, with recognition sequences for tobacco etch virus (TEV) or thrombin cleavage. A stop codon was added after the last chain. The ring designs were ordered in the same manner, except here the C-term His-tag was kept in frame to reduce overall DNA synthesis length. An additional Strep-tag II (WSHPQFEK (SEQ ID NO: 123)) to allow for Strep-Tactin™ pull-down was added to the N-term of chain B for DHT01 arms and DHT01 variant. Chains for individual expression had either an N-term or C-term 6×His-tag. Genes were ordered from Integrated DNA Technologies (IDT) or Genscript.

### Protein Expression and Purification

Plasmids were transformed into either BL21(DE3) or Lemo21(DE3) E. coli cells using a 30 second heat shock protocol, added to autoinduction media⁴⁰, and incubated at 225 rpm for 20-22 hours at 37° C. Cell pellets were obtained by centrifugation at 4000×g for 15 minutes, resuspended in 30 ml of lysis buffer (25 mM Tris-HCl pH 8.0, 300 mM NaCl, 20 mM imidazole, with added protease inhibitor PMSF), lysed with a sonicator at 85% amplitude with 15 second on/off cycles for a total of 2 minutes and 30 seconds, and then spun in the centrifuge at 24,000×g for 30 minutes. Cleared lysate was poured over an Ni-NTA column pre-equilibrated with 3 column volume (CV) of lysis buffer, washed with wash buffer (25 mM Tris-HCl pH 8.0, 300 mM NaCl, 30 mM imidazole) at 2×10 CV, and eluted with elution buffer (25 mM Tris-HCl pH 8.0, 300 mM NaCl, 250 mM imidazole) at 6 CV. For Strep-tag purification, Strep-Tactin XT Superflow high capacity resin (IBA) was equilibrated with 2 CV of Buffer W (100 mM Tris-HCl pH 8.0, 150 mM NaCl, 1 mM EDTA), IMAC eluate was poured over, column was washed with 5 CV of Buffer W, and protein was eluted with 3 CV of Buffer BXT (100 mM Tris-HCl, pH 8.0; 150 mM NaCl; 1 mM EDTA; 50 mM biotin). For TEV or thrombin cleavage, imidazole was cleared out through a buffer exchange into TBS buffer (25 mM Tris-HCl pH 8, 150 mM NaCl) and enzyme was applied for overnight cleavage. PMSF was used to stop thrombin cleavage and a second IMAC pulldown was carried out for either cleavage reaction. Flow-through was collected and run through SEC.

### SDS-PAGE

Protein samples were mixed with 2× Laemmli Sample Buffer, heated for 10 minutes at 95° C., and loaded onto Tris-Glycine gels along with 5 μL of BioRad's Precision Plus Protein Dual Xtra™ Protein Standard. Gel was run for 30 min at 200 V (Tris-Glycine) and then stained with Genscript's eStain™.

### Reconstitution Via Annealing

Separately expressed and individually purified components of the heterotrimer can be mixed at a 1:1:1 ratio in a PCR tube and incubated in a thermocycler. The mixture undergoes ˜30 minutes of heating at 90° C., followed by a gradual cooling by a 2° C. drop every 30 seconds until 12° C. is reached (resulting in a total of 20 minutes). For DHT03_2arm_A21/B21/C and subsequent 3-arm heterotrimers, 100 uM of each chain was mixed together for reconstitution, while ˜30 uM of each chain was mixed together for the DHT03 cyclic rings.

### Size Exclusion Chromatography (SEC)

An AKTA PURE FPLC system was used. Heterotrimer bases, arm extensions, coiled coil C2 rings, and all constructs mentioned in SI were passed through a Cytvia Superdex S200 Increase 10/300 GL column, while C3/C4 rings made from DHT03 were passed through a Cytvia Superdex S6 Increase 10/300 GL column. The mobile phase was TBS (25 mM Tris-HCl pH 8.0, 100 mM NaCl or 25 mM Tris-HCl pH 8.0, 150 mM NaCl). Samples ran at a flow rate of 0.75 ml/min and fractions were collected at 0.5 ml.

Mass Spectrometry The fraction corresponding to the SEC peak was concentrated to 1-2 mg/ml and run through Agilent 6230 LC/MS TOF through an AdvanceBio RP desalting column. The mass of the proteins was determined using intact mass spectrometry in positive mode.

### Native Mass Spectrometry^{41,42}

Samples were analyzed by online buffer exchange native mass spectrometry (nMS) to evaluate sample purity and accurately determine oligomeric states⁴². Multiple instruments were used as the analyses were carried out over the duration of the protein design process. The mass spectrometers used for detection were a Q Exactive™ UHMR modified with a surface-induced dissociation device and an Exactive™ Plus EMR modified with a selection quadrupole and a surface-induced dissociation device (Thermo Fisher Scientific)⁴³. The liquid chromatography systems used for the buffer exchange include a Vanquish™ Duo UHPLC and a Dionex™ Ultimate 3000 HPLC (Thermo Fisher Scientific). A heated electrospray ionization source (HESI-II, Thermo Fisher Scientific) with a spray voltage of ˜4 kV was used for ionization. Protein samples stored in Tris buffer were injected (0.1-2 ug) onto the LC system and exchanged at a flow rate of 100-200 uL*min⁻¹ into 200 mM ammonium acetate (mobile phase) prior to ionization. Buffer exchange columns used include self-packed columns with P6 polyacrylamide gel (Bio-Rad Laboratories) and prototype buffer exchange columns provided by Thermo Fisher Scientific (Sunnyvale, CA). Instrument parameters were optimized to allow for ion transmission while minimizing unintentional ion activation. Higher-energy collisional dissociation (HCD) and source fragmentation voltages were used for de-adducting to allow for accurate mass determination. Frequently, collisional dissociation leading to non-covalent fragmentation was used to further validate oligomeric composition. Mass spectra were deconvolved and oligomeric assignments were made using UniDec v5⁴⁴.

### CD

Samples were run over SEC through PBS (Phosphate-Buffered Saline pH 7.4) buffer, concentrated to 0.25 mg/ml, and placed in a 1 mm pathlength cuvette. A JASCO-1500 was used for wavelength scans (190-260 nm) at 25° C., 75° C., 95° C., and final 25° C. Temperature melts from 25 to 95° C. were monitored at 222 nm.

### SAXS⁴⁵

Purified samples were run through 25 mM Tris, 150 mM NaCl, and 2% glycerol buffer for SEC. Samples were concentrated using a 10K molecular weight cutoff (MWCO) benchtop spin concentrator and flow-through from the concentrator was used as a buffer blank. A 1.5-2.5 mg/ml low concentration range and a 3-6 mg/ml high concentration range were used for shipping to the SIBYLS High Throughput SAXS Advanced Light Source in Berkeley, California. The X-ray wavelength (λ) was 1.27 Å and the sample-to-detector distance was 1.5 m, corresponding to a scattering vector q (q=4π sin θ/λ, where 2θ is the scattering angle) range of 0.01 to 0.3 Å⁻¹. A series of exposures was taken of each well, in equal sub-second time slices: 0.3 second exposures for 10 seconds resulting in 32 frames per sample. Collected data was processed using the SIBYLS SAXS FrameSlice server and analyzed using ScÅtter3. Scattering output was fit to the theoretical design model using the FoXS server.

### X-Ray Crystallography Preparation, Data Collection, and Analysis

### Crystallization and Structure Determination for DHT03

Purified DHT03 protein at a concentration of 40 mg/mL was used to conduct sitting drop, vapor-diffusion crystallization trials using the JCSG Core I-IV screens (NeXtal Biotechnologies). Crystals of DHT03 grew from drops consisting of 100 nL protein plus 100 nL of a reservoir solution consisting of 0.1M HEPES pH7.5, 20%(w/v) PEG8000 at 4° C., and were cryoprotected by supplementing the reservoir solution with 15% Ethylene glycol. Native diffraction data was collected at APS beamline APS-23-ID-B, indexed to P1 and reduced using XDS⁴⁸. The structure was phased by molecular replacement using Phaser™. The core of DHT03_2arm_A21/B21/C long was used as a search model. The best solution with TFZ score 5.8 in Phaser™ was autobuild by SHELXE and the solution with best CC of model-map (0.35) was obtained for Coot™ adjustment and refinement using Phenix™

### Crystallization and Structure Determination for DHT03_1Arm_A21/B/C

Purified DHT03_1arm_A21/B/C protein at a concentration of 30 mg/mL was used to conduct sitting drop, vapor-diffusion crystallization trials using the JCSG Core I-IV screens (NeXtal Biotechnologies). Crystals of DHTO3_1arm_A21/B/C grew from drops consisting of 100 nL protein plus 100 nL of a reservoir solution consisting of 1M LiCl, 0.1M Sodium citrate pH5, 20% (w/v) PEG6000 at 4° C., and were cryoprotected by supplementing the reservoir solution with 15% Ethylene glycol. Native diffraction data was collected at APS beamline APS-23-ID-B, indexed to P1 and reduced using XDS⁴⁸. The structure was phased by molecular replacement using Phaser™. The core region of a set of ˜50 lowest energy predicted models from Rosetta™ were used as search models. The arm region was rigid-body fitted in the density subsequently in Coot™ and refined using Phenix™. The following model building and refinement were done by Coot™ and Phenix™

### Crystallization and Structure Determination for DHT03_2Arm_A21/B21/C Long

Purified DHT03_2arm_A21/B21/C (long) protein at a concentration of 41 mg/mL was used to conduct sitting drop, vapor-diffusion crystallization trials using the JCSG Core I-IV screens (NeXtal Biotechnologies). Crystals of DHT03_2arm_A21/B21/C long grew from drops consisting of 100 nL protein plus 100 nL of a reservoir solution consisting of 2M (NH4)2SO4, 0.1M sodium acetate pH4.6 at 18° C., and were cryoprotected by supplementing the reservoir solution with 2.2M sodium malonate pH5. Native diffraction data was collected at APS beamline APS-23-ID-B, indexed to P1 and reduced using XDS⁴⁸. The structure was phased by molecular replacement using Phaser™. The chain A of a set of ˜49 lowest energy predicted models from Rosetta™ were used as search models. Several of these models gave clear solutions. Chain B and Chain C were fitted manually in Coot™ and rigid body refinement by Phenix™. The following model building and refinement were done by Coot™ and Phenix™.

### NS-EM Preparation, Data Collection, and Analysis

All SEC-purified samples were diluted to 0.008 mg/mL in TBS buffer, at pH 8.0. For each sample, copper grids (LaceyCarbon™, with 1 μm hole diameter and 5 μm hole spacing) were glow-discharged; 6 μL of diluted samples were applied to the grids, and left on for 8 seconds and then dried with blotter paper; three rounds of grid-staining with uranyl formate (6 μL, 2 mg/mL) were applied to each grid, and left to sit for 8 seconds before blotting; the grids were left to dry for 5 minutes.

NS-EM data acquisition was performed on an FEI Talos L120C transmission electron microscope (120 keV accelerating voltage, 2.7 mm spherical abberation), at a magnification of 92,000× and pixel size of 1.54 Å×1.54 Å. Data collection for selected samples was performed using Thermo Fisher Scientific EPU software. Micrographs were stored as *.mrc files for subsequent processing.

To process and analyze the data, the collected micrographs were processed and analyzed using the CryoSPARC V3 software suite.

### REFERENCES

1. Xu, Q. & Dunbrack, R. L. Principles and characteristics of biological assemblies in experimentally determined protein structures. Current Opinion in Structural Biology 55, 34-49 (2019).
2. Gauba, V. & Hartgerink, J. D. Surprisingly High Stability of Collagen ABC Heterotrimer: Evaluation of Side Chain Charge Pairs. J. Am. Chem. Soc. 129(48), 15034-15041 (2007).
3. Poirier, M. A. et al. The synaptic SNARE complex is a parallel four-stranded helical bundle. Nature Structural Biology 5(9), 765-769 (1998).
4. Macdonald, P. R., Lustig, A., Steinmetz, M. O., Kammerer, R. A. Laminin chain assembly is regulated by specific coiled-coil interactions. J Struct Biol. 170(2), 398-405 (2010).
5. Pandya, M. J., Spooner, G. M., Sunde, M., Thorpe, J. R., Rodger, A., Woolfson, D. N. Sticky-end assembly of a designed peptide fiber provides insight into protein fibrillogenesis. Biochemistry 39(30), 8728-34 (2000).
6. Moll, J. R., Ruvinov, S. B., Pastan, I., Vinson, C. Designed heterodimerizing leucine zippers with a ranger of pIs and stabilities up to 10⁻¹⁵ M. Protein Sci. 10, 649-655 (2001).
7. Arndt, K. M., Pelletier, J. N., Müller, K. M., Plückthun, A., Alber, T. Comparison of in vivo selection and rational design of heterodimeric coiled coils. Structure 10(9), 1235-48 (2002).
8. Bromley, E. H. C., Sessions, R. B., Thomson, A. R., Woolfson, D. N. Designed alpha-helical tectons for constructing multicomponent synthetic biological systems. J. Am. Chem. Soc. 131, 928-930 (2009).
9. Reinke, A. W., Grant, R. A., and Keating, A. E. A Synthetic Coiled-Coil Interactome Provides Heterospecific Modules for Molecular Engineering. J. Am. Chem. Soc. 132(17), 6025-6031 (2010).
10. Thompson, K. E., Bashor, C. J., Lim, W. A. & Keating, A. E. SYNZIP protein interaction toolbox: in vitro and in vivo specifications of heterospecific coiled-coil interaction domains. ACS Synth. Biol. 1, 118-129 (2012).
11. Fletcher, J. M. et al. Self-assembling cages from coiled-coil peptide modules. Science 340, 595-599 (2013).
12. Chen, Z. et al. Programmable design of orthogonal protein heterodimers. Nature 565(7737), 106-111 (2019).
13. Chen, Z. et al. De novo design of protein logic gates. Science 368(6486), 78-84 (2020).
14. Nautiyal, S., Woolfson, D. N., King, D. S., Alber, T. A designed heterotrimeric coiled coil. Biochemistry 34(37), 11645-11651 (1995).
15. Nautiyal, S. & Alber, T. Crystal structure of a designed, thermostable, heterotrimeric coiled coil. Prot. Sci. 8, 84-90 (1999).
16. Lombardi, A., Bryson, J. W. & DeGrado, W. F. De novo design of heterotrimeric coiled coils. Biopolymers 40, 495-504 (1996).
17. O'Shea, E. K., Lumb, K. J., Kim, P. S. Peptide ‘Velcro’: design of a heterodimeric coiled coil. Curr Biol. 3(10), 658-667 (1993).
18. Kashiwada, A., Hiroaki, H., Kohda, D., Nango, M., Tanaka, T. Design of a Heterotrimeric α-Helical Bundle by Hydrophobic Core Engineering. J. Am. Chem. Soc. 122(2), 212-215 (2000).
19. Kiyokawa, T. et al. Selective formation of AAB- and ABC-type heterotrimeric alpha-helical coiled coils. Chemistry 10(14), 3548-54 (2004).
20. Schnarr, N. A. & Kennan, A. J. Peptide Tic-Tac-Toe: Heterotrimeric Coiled-Coil Specificity from Steric Matching of Multiple Hydrophobic Side Chains. J. Am. Chem. Soc. 124(33), 9779-9783 (2002).
21. Schnarr, N. A. & Kennan, A. J. Strand orientation by steric matching: a designed antiparallel coiled-coil trimer. J. Am. Chem. Soc. 126(44), 14447-14451 (2004).
22. Fleishman, S. J. & Baker, D. Role of the Biomolecular Energy Gap in Protein Design, Structure, and Evolution. Cell 149(2), 262-273 (2012).
23. Crick, F. H. C. The Fourier transform of a coiled-coil. Acta Cryst 6, 685-689 (1953).
24. Grigoryan, G. & Degrado, W. F. Probing designability via a generalized model of helical bundle geometry. J. Mol. Biol. 405, 1079-1100 (2011).
25. Maguire, J. B., Boyken, S. E., Baker, D., Kuhlman, B. Rapid Sampling of Hydrogen Bond Networks for Computational Protein Design. J. Chem. Theory Comput. 14(5), 2751-2760 (2018).
26. Boyken, S. E. et al. De novo design of protein homo-oligomers with modular hydrogen-bond network-mediated specificity. Science 352, 680-687 (2016).
27. Alford, R. F. et al. The Rosetta All-Atom Energy Function for Macromolecular Modeling and Design. J. Chem. Theory Comput. 13(6), 3031-3048 (2017).
28. Coventry, B. & Baker, D. Protein sequence optimization with a pairwise decomposable penalty for buried unsatisfied hydrogen bonds. PLoS Comput Biol 17(3), e1008061 (2021).
29. Hiranuma, N. & Park, H. et al. Improved protein structure refinement guided by deep learning based accuracy estimation. Nature Communications 12, 1340 (2021).
30. Brunette, T. J., Parmeggiani, F., Huang, P. S. et al. Exploring the repeat protein universe through computational protein design. Nature 528, 580-584 (2015).
31. Hsia, Y. & Mout, R. et al. Design of multi-scale protein complexes by hierarchical building block fusion. Nature Communications 12, 2294 (2021).
32. Brunette, T. J. et al. Modular repeat protein sculpting using rigid helical junctions. Proc. Natl Acad. Sci. USA 117, 8870-8875 (2020).
33. Lauer, T. M. et al. Developability index: a rapid in silico tool for the screening of antibody aggregation propensity. J Pharm Sci 101(1), 102-115 (2012).
34. Evans, R. et al. Protein complex prediction with AlphaFold-Multimer. Biorxiv. (2021).
35. Jumper, J. et al. Highly accurate protein structure prediction with AlphaFold. Nature 596, 583-589 (2021).
36. Vulovic I. et al. Generation of ordered protein assemblies using rigid three-body fusion. PNAS 118(23), e2015037118 (2021).
37. Sahtoe, D. D. & Praetorius, F. et al. Reconfigurable asymmetric protein assemblies through implicit negative design. Science 375(6578), eabj7662 (2022).
38. Yeates, T. O., Liu, Y. & Laniado, J. The design of symmetric protein nanomaterials comes of age in theory and practice. Curr. Opin. Struct. Biol. 39, 134-143 (2016).
39. Rocklin, G. J. et al. Global analysis of protein folding using massively parallel design, synthesis and testing. Science 357(6347), 168-175 (2017).
40. Studier F. W. Protein production by auto-induction in high density shaking cultures. Protein Expr Purif 41(1), 207-234 (2005).
41. Sahasrabuddhe, A et al. Confirmation of intersubunit connectivity and topology of designed protein complexes by native MS. PNAS 115(6), 1268-1273 (2018).
42. VanAernum, Z. L. et al. Rapid online buffer exchange for screening of proteins, protein complexes and cell lysates by native mass spectrometry. Nature Protocols 15, 1132-1157 (2020).
43. VanAernum, Z. L. et al. Surface-induced dissociation of noncovalent protein complexes in an extended mass range orbitrap mass spectrometer. Anal. Chem. 91, 3611-3618 (2019).
44. Marty, M. T. et al. Bayesian deconvolution of mass and ion mobility spectra: from binary interactions to polydisperse ensembles. Anal. Chem. 87, 4370-4376 (2015).
45. Dyer, K. N. et al. High-throughput SAXS for the characterization of biomolecules in solution: a practical approach. Methods Mol. Biol. 1091, 245-258 (2014).
46. Schneidman-Duhovny, D., Hammel, M., Tainer, J. A., and Sali, A. Accurate SAXS profile computation and its assessment by contrast variation experiments. Biophysical Journal 105 (4), 962-974 (2013).
47. Schneidman-Duhovny, D., Hammel, M., Tainer, J. A., and Sali, A. FoXS, FoXSDock and MultiFoXS: Single-state and multi-state structural modeling of proteins and their complexes based on SAXS profiles. NAR 44(W1), W424-W429 (2016).
48. Kabsch, W. XDS. Acta Crystallogr. D Biol. Crystallogr. 66, 125-132 (2010).
49. McCoy, A. J. et al. Phaser crystallographic software. J. Appl. Crystallogr. 40, 658-674 (2007).
50. Emsley, P., Lohkamp, B., Scott, W. G. & Cowtan, K. Features and development of Coot. Acta Crystallogr. D Biol. Crystallogr. 66, 486-501 (2010).
51. Adams, P. D. et al. PHENIX: a comprehensive Python-based system for macromolecular structure solution. Acta Crystallogr. D Biol. Crystallogr. 66, 213-221 (2010).
52. Punjani, A., Rubinstein, J. L., Fleet, D. J., Brubaker, M. A. cryoSPARC: algorithms for rapid unsupervised cryo-EM structure determination. Nature Methods 14(3), 290-296 (2017).
53. Fallas, J. A., Ueda, G., Sheffler, W. et al. Computational Design of Self-Assembling Cyclic Protein Homo-oligomers. Nat Chem. 9(4), 353-360 (2017).
54. DiMaio, F., Tyka, M. D., Baker, M. L., Chiu, W, Baker, D. Refinement of protein structures into low-resolution density maps using Rosetta. Journal of Molecular Biology 392(1), 181-190 (2009).

1. A polypeptide comprising an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of the amino acid sequences listed in Tables 1, 2, and 3, wherein 0-7 residues at the N and/or C-terminus are optional and may be absent and not considered when determining percent identity.

2. The polypeptide of claim 1, wherein interface residues in the reference sequence as identified in Table 4 are maintained.

3. The polypeptide of claim 1, wherein residues capable of hydrogen-bonding as identified in Table 4 are maintained.

4. The polypeptide of claim 1, wherein W, Y, and F residues in the reference sequence are maintained.

5. The polypeptide of claim 1, wherein mutations in residues relative to the reference sequence are conservative amino acid substitutions

6. A fusion protein comprising
(a) the polypeptide of claim 1;
(b) a second polypeptide; and
(c) an optional amino acid linker linking the polypeptide and second polypeptide.

7. The fusion protein of claim 6, wherein the second polypeptide comprises a helical repeat protein or a protein with mixed alpha helix/beta sheet secondary structure.

8. The fusion protein of claim 6, wherein the fusion protein comprises an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of sequences in Tables 5 and 6.

9. A polypeptide comprising an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of sequences in Tables 5 and 6.

10. A nucleic acid encoding the polypeptide of claim 1.

11. An expression vector comprising the nucleic acid of claim 10 operatively linked to a suitable control element.

12. A host cell comprising expression vector of claim 11.

13. A heterotrimer, heterodimer, or heterotetramer comprising polypeptides that comprise an amino acid sequence at least 70%, 75%, 80%, 85%, 90%, 91%, 92%, 93%, 94%, 95%, 96%, 97%, 98%, 99%, or 100% identical to an amino acid sequence combinations listed in Table 1, Table 2, Table 3, Table 5, or Table 6, wherein 0-7 residues at the N and/or C-terminus of the polypeptides are optional and may be absent and not considered when determining percent identity.

14. The heterotrimer, heterodimer, or heterotetramer of claim 13, wherein the heterotrimer, heterodimer, or heterotetramer comprises an interaction hub building block between chains in larger closed structures.

15. A kit comprising one or more polypeptide of claim 1.

16. A method for using the polypeptide of claim 1 for any suitable purpose, including but not limited to antigen presentation.

17. A method for computational design of the polypeptide of claim 1, comprising any method or steps as disclosed in the attached appendices.
